(19) United States
(12) Patent Application Publication
(10) Pub. No.: US 2025/0009882 A1
(43) Pub. Date: Jan. 9, 2025

(54) ENGINEERED EFFECTOR CELLS FOR TRAFFICKING OF ALLOGENEIC CELL THERAPIES IN SOLID TUMORS

(71) Applicant: Fate Therapeutics, Inc., San Diego, CA (US)

(72) Inventors: Martin HOSKING, San Diego, CA (US); Bahram VALAMEHR, San Diego, CA (US); Soheila SHIRINBAK, San Diego, CA (US)

(21) Appl. No.: 18/706,956

(22) PCT Filed: Nov. 7, 2022
(86) PCT No.: PCT/US2022/079412

§ 371 Date: May 2, 2024

Related U.S. Application Data
(60) Provisional application No. 63/277,034, filed on Nov. 8, 2021.
(60) Provisional application No. 63/329,203, filed on Apr. 8, 2022.

(51) Int. Cl. A61K 39/00; C07K 14/715; C07K 14/725; C12N 9/22; C12N 15/11; C12N 15/90

(52) U.S. Cl. CPC A61K 39/4635; A61K 39/4611; A61K 39/4631; C07K 14/7051; C07K 14/7155; C12N 9/22; C12N 15/11; C12N 15/907; C12N 2310/20

(57) ABSTRACT

Provided are methods and compositions including for obtaining functionally enhanced derivative effector cells obtained from directed differentiation of genomically engineered iPSCs. Also provided are derivative cells having stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### RELATED APPLICATIONS

This application claims priority to U.S. Provisional Application Ser. No. 63/277,034, filed Nov. 8, 2021, and to U.S. Provisional Application Ser. No. 63/329,203, filed Apr. 8, 2022, the disclosure of each of which is hereby incorporated by reference in their entireties.

### INCORPORATION BY REFERENCE OF SEQUENCE LISTING

The Sequence Listing titled 184143-638601_SL.xml, which was created on Nov. 6, 2022 and is 33,429 bytes in size, is hereby incorporated by reference in its entirety.

### FIELD OF THE INVENTION

The present disclosure is broadly concerned with the field of off-the-shelf immunocellular products. More particularly, the present disclosure is concerned with the strategies for developing multifunctional effector cells capable of delivering therapeutically relevant properties in vivo. Cell products developed under the present disclosure address critical limitations of patient-sourced cell therapies.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit therefrom. There is also the need to improve the efficacy and persistence of adoptively transferred lymphocytes to promote favorable patient outcomes. Lymphocytes such as T cells and natural killer (NK) cells are potent anti-tumor effectors that play an important role in innate and adaptive immunity. However, the use of these immune cells for adoptive cell therapies remains challenging and has unmet needs for improvement. Therefore, there remain significant opportunities to harness the full potential of T and NK cells, or other lymphocytes in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, i.e., tumor microenvironment and related immune suppression, recruiting, trafficking and infiltration.

It is an object of embodiments of the present invention to provide methods and compositions to generate derivative non-pluripotent cells differentiated from a single cell derived iPSC (induced pluripotent stem cell) clonal line, which iPSC line comprises one or several genetic modifications in its genome. Said one or several genetic modifications include, in some embodiments, DNA insertion, deletion, and substitution, and which modifications are retained and remain functional in subsequently derived cells after differentiation, expansion, passaging and/or transplantation.

The iPSC-derived non-pluripotent cells of the present application include, but are not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells. The iPSC-derived non-pluripotent cells of the present application comprise one or several genetic modifications in their genome through differentiation from an iPSC comprising the same genetic modifications. In some embodiments, the engineered clonal iPSC differentiation strategy for obtaining genetically engineered derivative cells benefits from the developmental potential of the iPSC in a directed differentiation that is not significantly impacted by the engineered modality in the iPSC, and also that the engineered modality functions as intended in the derivative cell. Further, this strategy overcomes the present barrier in engineering primary lymphocytes, such as T cells or NK cells obtained from peripheral blood, as such cells are difficult to engineer, with engineering of such cells often lacking reproducibility and uniformity, resulting in cells exhibiting poor cell persistence with high cell death and low cell expansion. Moreover, this strategy avoids production of a heterogenous effector cell population otherwise obtained using primary cell sources which are heterogenous to start with.

Some aspects of the present invention provide genome-engineered iPSCs obtained using a method comprising (I), (II) or (III), reflecting a strategy of genomic engineering subsequently to, simultaneously with, and prior to the reprogramming process, respectively:
(I): genetically engineering iPSCs by one or both of (i) and (ii), in any order: (i) introducing into iPSCs one or more construct(s) to allow targeted integration at selected site(s); (ii) (a) introducing into iPSCs one or more double stranded break(s) at selected site(s) using one or more endonuclease capable of selected site recognition; and (b) culturing the iPSCs of step (I)(ii)(a) to allow endogenous DNA repair to generate targeted in/dels at the selected site(s); thereby obtaining genome-engineered iPSCs capable of differentiation into partially or fully differentiated cells.
(II): genetically engineering reprogramming non-pluripotent cells to obtain the genome-engineered iPSCs comprising: (i) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate reprogramming of the non-pluripotent cells; and (ii) introducing into the reprogramming non-pluripotent cells of step (II)(i) one or both of (a) and (b), in any order: (a) one or more construct(s) to allow targeted integration at selected site(s); (b) one or more double stranded break(s) at a selected site using at least one endonuclease capable of selected site recognition, then the cells of step (II)(ii)(b) are cultured to allow endogenous DNA repair to generate targeted in/dels at the selected site(s); as such the obtained genome-engineered iPSCs comprise at least one functional targeted genomic editing, and said genome-engineered iPSCs are capable of differentiation into partially or fully differentiated cells.
(III): genetically engineering non-pluripotent cells for reprogramming to obtain genome-engineered iPSCs comprising (i) and (ii): (i) introducing into non-pluripotent cells one or both of (a) and (b), in any order: (a) one or more construct(s) to allow targeted integration at selected site(s); (b) one or more double stranded break(s) at a selected site using at least one endonuclease capable of selected site recognition, wherein the cells of step (III)(i)(b) are cultured to allow endogenous DNA repair to generate targeted in/dels at the selected sites; and (ii) contacting the cells of step (III)(i) with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted editing at selected sites; thereby obtaining genome-engineered iPSCs comprising at least one functional targeted genomic editing, and said genome-engineered iPSCs are capable of being differentiated into partially differentiated cells or fully-differentiated cells.

In one embodiment of the above method, the at least one targeted genomic editing at one or more selected sites comprises insertion of one or more exogenous polynucleotides encoding safety switch proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof. In some embodiments, the exogenous polynucleotides for insertion are operatively linked to (1) one or more exogenous promoters comprising CMV, EF1α, PGK, CAG, UBC, or other constitutive, inducible, temporal-, tissue-, or cell type-specific promoters; or (2) one or more endogenous promoters comprised in the selected sites comprising AAVS1, CCR5, ROSA26, collagen, HTRP, H11, beta-2 microglobulin, GAPDH, TCR or RUNX1, or other locus meeting the criteria of a genome safe harbor. In some embodiments, the genome-engineered iPSCs generated using the above method comprise one or more different exogenous polynucleotides encoding protein comprising caspase, thymidine kinase, cytosine deaminase, modified EGFR, or B-cell CD20, wherein when the genome-engineered iPSCs comprise two or more suicide genes, the suicide genes are integrated in different safe harbor locus comprising AAVS1, CCR5, ROSA26, collagen, HTRP, H11, H11, beta-2 microglobulin, GAPDH, TCR or RUNX1. In one embodiment, the exogenous polynucleotide encodes a partial or full length peptide of IL2, IL4, IL6, IL7, IL9, IL10, IL11, IL12, IL15, IL18, IL21, and/or of respective receptors thereof. In some embodiments, the partial or full length peptide of IL2, IL4, IL6, IL7, IL9, IL10, IL11, IL12, IL15, IL18, IL21, and/or of respective receptors thereof encoded by the exogenous polynucleotide is in a form of fusion protein.

In some other embodiments, the genome-engineered iPSCs generated using the method provided herein comprise in/del at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof. In some embodiments, the endogenous gene for disruption comprises at least one of B2M, TAP1, TAP2, Tapasin, NLRC5, PD1, LAG3, TIM3, RFXANK, CIITA, RFX5, RFXAP, and any gene in the chromosome 6p21 region.

In yet some other embodiments, the genome-engineered iPSCs generated using the method provided herein comprise a caspase encoding exogenous polynucleotide at AAVS1 locus, and a thymidine kinase encoding exogenous polynucleotide at H11 locus.

In still some other embodiments, approach (I), (II) and/or (III) further comprises: contacting the genome-engineered iPSCs with a small molecule composition comprising a MEK inhibitor, a GSK3 inhibitor and a ROCK inhibitor, to maintain the pluripotency of the genomic-engineered iPSCs. In one embodiment, the obtained genome engineered iPSCs comprising at least one targeted genomic editing are functional, are differentiation potent, and are capable of differentiating into non-pluripotent cells comprising the same functional genomic editing.

Accordingly, in one aspect, the present invention also provides a composition comprising a cell or population thereof, wherein: (i) the cell is (a) an immune cell; (b) an induced pluripotent cell (iPSC); or (c) a derivative cell obtained from differentiating the iPSC; (ii) the cell comprises: (1) an exogenous polynucleotide encoding CXCR2 or a variant thereof, and optionally a chimeric antigen receptor (CAR). In various embodiments of the cell or population thereof, the iPSC is a clonal iPSC, a single cell dissociated iPSC, an iPSC cell line cell, or an iPSC master cell bank (MCB) cell; or the derivative cell comprises (i) the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, or a derivative B lineage cell; or (ii) the derivative cell comprises a derivative effector cell having one or more functional features that are not present in a counterpart primary T, NK, NKT, and/or B cell.

In various embodiments of the cell or population thereof, the cell further comprises one or more of: (i) CD38 knockout; (ii) HLA-I deficiency and/or HLA-II deficiency; (iii) introduction of HLA-G or non-cleavable HLA-G, or knockout of one or both of CD58 and CD54; (iv) an exogenous CD16 or a variant thereof, (v) a cytokine signaling complex comprising a partial or full peptide of a cell surface expressed exogenous cytokine and/or a receptor thereof; (vi) at least one of the genotypes listed in Table 1; (vii) disruption of least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (viii) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, an engager, and surface triggering receptor for coupling with an agonist. In various embodiments, the cell has therapeutic properties comprising one or more of: (i) increased cytotoxicity; (ii) improved persistency and/or survival; (iii) enhanced ability in migrating, and/or activating or recruiting bystander immune cells, to tumor sites; (iv) improved tumor penetration; (v) enhanced ability to reduce tumor immunosuppression; (vi) improved ability in rescuing tumor antigen escape; (vii) controlled apoptosis; (viii) enhanced or acquired ADCC; and (ix) ability to avoid fratricide, in comparison to its counterpart primary cell obtained from peripheral blood, umbilical cord blood, or any other donor tissues without the same genetic edit(s).

In some embodiments of the cell or population thereof, the exogenous CD16 or variant thereof comprises (a) a high affinity non-cleavable CD16 (hnCD16) or a variant thereof, (b) F176V and S197P in ectodomain domain of CD16; (c) a full or partial ectodomain originated from CD64; (d) a non-native (or non-CD16) transmembrane domain; (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide. In some embodiments of the cell or population thereof, the CAR is: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) co-expressed with another CAR; (ix) co-expressed with a cytokine signaling complex comprising a partial or full peptide of a cell surface expressed exogenous cytokine and/or a receptor thereof, optionally in separate constructs or in a bi-cistronic construct; (x) co-expressed with a checkpoint inhibitor, optionally in separate constructs or in a bi-cistronic construct; (xi) specific to at least one of CD19, BCMA, CD20, CD22, CD38, CD123, HER2, CD52, EGFR, GD2, MICA/B, MSLN, VEGF-R2, PSMA and PDL1; and/or (xii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A_{2A}, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, Mucin 1 (Muc-1), Mucin 16 (Muc-16), Mesothelin (MSLN), NKCSI, NKG2D ligands, c-Met, cancer-testis antigen NY-ESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and optionally, wherein the CAR of any one of (i) to (xii) is inserted at a TCR locus, and/or is driven by an endogenous promoter of the TCR, and/or the TCR is knocked out by the CAR insertion.

In some embodiments of the cell or population thereof, the cytokine signaling complex: (a) comprises a partial or full peptide of at least one of IL2, IL4, IL6, IL7, IL9, IL10, IL11, IL12, IL15, IL18, IL21, and respective receptor thereof; or (b) comprises at least one of: (i) co-expression of IL15 and IL15Rα by suing a self-cleaving peptide; (ii) a fusion protein of IL15 and IL15Rα; (iii) an IL15/IL15Rα fusion protein with intracellular domain of IL15Rα truncated; (iv) a fusion protein of IL15 and membrane bound Sushi domain of IL15Rα; (v) a fusion protein of IL15 and IL15Rβ; (vi) a fusion protein of IL15 and common receptor TC, wherein the common receptor γC is native or modified; and (vii) a homodimer of IL5Rβ; wherein any one of (b)(i)-(vii) can be co-expressed with a CAR in separate constructs or in a bi-cistronic construct; or (c) comprises at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with a CAR in separate constructs or in a bi-cistronic expression cassette; and optionally, (d) is transiently expressed. In some embodiments of the cell or population thereof, the cell is an NK lineage cell or a T lineage cell, wherein (i) the NK lineage cell or the T lineage cell has improved infiltration and/or retention at tumor sites; (ii) the NK lineage cell is capable of recruiting, and/or migrating T cells to tumor sites; or (iii) the NK lineage cell or the T lineage cell is capable of reducing tumor immunosuppression in the presence of one or more checkpoint inhibitors.

In various embodiments, the checkpoint inhibitors are antagonists to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR. In certain embodiments, the checkpoint inhibitors comprise: (a) one or more of atezolizumab, avelumab, durvalumab, ipilimumab, IPH4102, IPH43, IPH33, lirimumab, monalizumab, nivolumab, pembrolizumab, and their derivatives or functional equivalents; or (b) at least one of atezolizumab, nivolumab, and pembrolizumab.

In some embodiments of the cell or population thereof, the cell comprises: (i) one or more exogenous polynucleotides integrated in a safe harbor locus or a selected gene locus; or (ii) more than two exogenous polynucleotides integrated in different safe harbor loci or two or more selected gene loci. In certain embodiments, the safe harbor locus comprises at least one of AAVS1, CCR5, ROSA26, collagen, HTRP, H11, GAPDH, or RUNX1; or wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CD69, CD71, CIS, CBL-B, SOCS2, PD1, CTLA4, LAG3, TIM3, or TIGIT; and/or wherein the integration of the exogenous polynucleotides knocks out expression of the gene in the locus. In some embodiments, the TCR locus is a constant region of TCR alpha and/or TCR beta.

In another aspect, the application provides a method for improving T lineage cell migration and/or tumor cell control and clearance, the method comprising obtaining a T lineage cell comprising an exogenous polynucleotide encoding CXCR2 or a variant thereof, and expanding the T lineage cell, thereby producing a T lineage cell population having improved cell migration and/or tumor cell control and clearance as compared to counterpart cells without the exogenous polynucleotide encoding CXCR2 or variant thereof, wherein the T lineage cell optionally further comprises a chimeric antigen receptor (CAR). In some embodiments of the method, the step of obtaining comprises (I) or (II): (I)(i) engineering an induced pluripotent cell (iPSC) to produce a genomically edited iPSC that comprises one or more exogenous polynucleotides encoding CXCR2 or a variant thereof and optionally a CAR; and (ii) differentiating the genomically edited iPSC to a derivative T lineage cell comprising the CXCR2 or variant thereof and optionally the CAR; or (II) engineering a T lineage cell by introducing a polynucleotide encoding CXCR2 or a variant thereof to the T lineage cell to produce a genomically edited T lineage cell that comprises the CXCR2 or a variant thereof and optionally the CAR. In various embodiments of the method, the genomically edited iPSC further comprises one or more edits resulting in: (i) CD38 knockout; (ii) HLA-I deficiency and/or HLA-II deficiency; (iii) introduction of HLA-G or non-cleavable HLA-G, or knockout of one or both of CD58 and CD54; (iv) introduction of an exogenous CD16 or a variant thereof, (v) a cytokine signaling complex comprising a partial or full peptide of a cell surface expressed exogenous cytokine and/or a receptor thereof, (vi) at least one of the genotypes listed in Table 1; (vii) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (viii) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, an engager, and surface triggering receptor for coupling with an agonist, in comparison to its counterpart primary cell obtained from peripheral blood, umbilical cord blood, or any other donor tissues without the same genomic edit(s). In various embodiments, the improved cell migration and/or tumor cell control and clearance is in vitro and/or in vivo.

In another aspect, the application provides a method of improving CAR-T cell in vivo antitumor function according to the methods provided herein.

In another aspect, the application provides a composition comprising the cell or population thereof provided herein. In various embodiments, the composition further comprises one or more therapeutic agents. In particular embodiments, the one or more therapeutic agents comprise a peptide, a cytokine, a checkpoint inhibitor, an effector, an antibody or functional variant or fragment thereof, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD).

In those embodiments of the composition where the therapeutic agent is a checkpoint inhibitor, the checkpoint inhibitor may comprise: (i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR; (ii) one or more of atezolizumab, avelumab, cetuximab, durvalumab, ipilimumab, IPH4102, IPH43, IPH33, lirimumab, monalizumab, nivolumab, pembrolizumab, trastuzumab, pertuzumab, and their derivatives or functional equivalents; (iii) at least one of atezolizumab, nivolumab, and pembrolizumab; or the therapeutic agents comprise one or more of venetoclax, azacitidine, and pomalidomide. In those embodiments where the therapeutic agent is an antibody or functional variant or fragment thereof, the antibody or functional variant or fragment thereof may comprise: (a) an anti-CD20, an anti-HER2, an anti-CD52, an anti-EGFR, an anti-CD123, an anti-GD2, an anti-PDL1, and/or an anti-CD38 antibody; or (b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or (c) daratumumab, and wherein the derivative effector cell comprises a CD38 knockout, and optionally an expression of CD16 or a variant thereof.

In various embodiments of the composition, the composition further comprises a sensitizing agent. In various embodiments, the sensitizing agent comprises at least one of a chemotherapeutic agent, external beam radiation, brachytherapy, and a radiopharmaceutical. In various embodiments, the the sensitizing agent increases chemokine secretion and/or surface expression by a tumor cell upon contact therewith. In certain embodiments, the sensitizing agent comprises: (i) at least one of calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iorn-59, krypton-81m, lutetium-177, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90; (ii) paclitaxel; or (iii) cyclophosphamide and fludarabine (Cy/Flu).

In another aspect, the invention provides therapeutic use of compositions described herein by introducing the composition to a subject suitable for adoptive cell therapy, wherein the subject has an autoimmune disorder; a hematological malignancy; a solid tumor; cancer, or a virus infection. In yet another aspect, the application provides a master cell bank (MCB) comprising a clonal iPSC provided herein.

In yet another aspect, the invention provides a method of manufacturing a derivative effector cell comprising CXCR2 or variant thereof, wherein the method comprises differentiating a genetically engineered iPSC to the derivative effector cell, wherein the genetically engineered iPSC comprises an exogenous polynucleotide encoding the CXCR2 or variant thereof and optionally one or more edits resulting in: (i) a CAR; (ii) exogenous CD16 or a variant thereof, (iii) CD38 knockout; (iv) HLA-I deficiency and/or HLA-II deficiency; (v) introduction of HLA-G or non-cleavable HLA-G, or knockout of one or both of CD58 and CD54; (vi) a cytokine signaling complex comprising a partial or full peptide of a cell surface expressed exogenous cytokine and/or a receptor thereof, (vii) at least one of the genotypes listed in Table 1; (viii) disruption of least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (ix) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, an engager, and surface triggering receptor for coupling with an agonist. In various embodiments of the method of manufacturing, the iPSC is a clonal iPSC, a single cell dissociated iPSC, an iPSC cell line cell, or an iPSC master cell bank (MCB) cell; or the derivative cell comprises (i) the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, or a derivative B lineage cell; or (ii) the derivative cell comprises a derivative effector cell having one or more functional features that are not present in a counterpart primary T, NK, NKT, and/or B cell. In various embodiments, the exogenous CD16 or a variant thereof comprises at least one of: (a) a high affinity non-cleavable CD16 (hnCD16); (b) F176V and S197P in ectodomain domain of CD16; (c) a full or partial ectodomain originated from CD64; (d) a non-native (or non-CD16) transmembrane domain; (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide. In some embodiments, the CAR is: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) co-expressed with another CAR; (ix) co-expressed with a cytokine signaling complex comprising a partial or full peptide of a cell surface expressed exogenous cytokine and/or a receptor thereof, optionally in separate constructs or in a bi-cistronic construct; (x) co-expressed with a checkpoint inhibitor, optionally in separate constructs or in a bi-cistronic construct; (xi) specific to at least one of CD19, BCMA, CD20, CD22, CD38, CD123, HER2, CD52, EGFR, GD2, MICA/B, MSLN, VEGF-R2, PSMA and PDL1; and/or (xii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb □B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, Mucin 1 (Muc-1), Mucin 16 (Muc-16), Mesothelin (MSLN), NKCSI, NKG2D ligands, c-Met, cancer-testis antigen NY-ESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and optionally, wherein the CAR of any one of (i) to (xii) is inserted at a TCR locus, and/or is driven by an endogenous promoter of TCR, and/or the TCR is knocked out by the CAR insertion.

In various embodiments of the method of of manufacturing, the method further comprises genomically engineering a clonal iPSC to knock-in a polynucleotide encoding the CXCR2 or variant thereof, and optionally: (i) to knock out CD38, (ii) to knock out B2M and/or CIITA, (iii) to knock out one or both CD58 and CD54, and/or (iv) to introduce one or more of HLA-G or non-cleavable HLA-G, an exogenous CD16 or a variant thereof, a CAR, and/or a cytokine signaling complex comprising a partial or full peptide of a cell surface expressed exogenous cytokine and/or a receptor thereof. In particular embodiments, the genomic engineering comprises targeted editing. In some embodiments, the targeted editing comprises deletion, insertion, or in/del, and wherein the targeted editing is carried out by CRISPR, ZFN, TALEN, homing nuclease, homology recombination, or any other functional variation of these methods.

In another aspect, the application provides a method of producing a clonal master engineered iPSC line using CRISPR mediated editing of clonal iPSCs, wherein the editing comprises a knock-in of a polynucleotide encoding CXCR2 or variant thereof and optionally a CAR, thereby producing the engineered iPSCs. In various embodiments of the method: (a) the editing of clonal iPSCs further comprises knocking out TCR, or (b) the CXCR2 or variant thereof is inserted at one of the gene loci comprising: B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and wherein the insertion knocks out expression of the gene in the locus. In various embodiments of the method, the method further comprises sorting the engineered iPSCs to obtain single cell dissociated iPSCs comprising the polynucleotide encoding CXCR2 or variant thereof and optionally the CAR. In some embodiments of the method, the method further comprises expanding the single cell dissociated iPSCs to produce the clonal engineered iPSC population. In some embodiments of the method, the method further comprises cryopreserving the produced clonal master engineered iPSC line. In particular embodiments of the method, the method further comprises analyzing off-target edits and/or karyotype of the engineered iPSCs. In another aspect, the invention provides a clonal master engineered iPSC line produced using the methods described herein.

In another aspect, the invention provides a method of treating a disease or a condition comprising administering to the subject in need thereof the compositions described herein. In various embodiments, the method comprises ad mistering to the subject in need thereof the cell or population thereof described herein. In various embodiments, the method further comprises administering a sensitizing agent to the subject, thereby preconditioning tumor cells in the subject. In various embodiments, the method further comprises administering one or more therapeutic agents to the subject.

In another aspect, the application provides a method of treating a subject comprising: (a) administering a sensitizing agent a tumor cell in to the subject to precondition tumor cells in the subject; and (b) administering the cell or population thereof described herein to the subject following administration of the sensitizing agent, wherein the subject has an autoimmune disorder; a hematological malignancy; a solid tumor; cancer, or a virus infection. In some embodiments of the method of treating, the sensitizing agent comprises at least one of a chemotherapeutic agent, external beam radiation, brachytherapy, and a radiopharmaceutical. In some embodiments of the method of treating, the sensitizing agent comprises: (i) at least one of x-ray radiation, gamma radiation, photon radiation, proton radiation, and neutron radiation; or (ii) at least one of calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iorn-59, krypton-81m, lutetium-177, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90; or (iii) paclitaxel; or (iv) cyclophosphamide and fludarabine (Cy/Flu).

In various embodiments of the method of treating, the method further comprises administering one or more therapeutic agents. In some embodiments of the method of treating, the one or more therapeutic agents comprise a peptide, a cytokine, a checkpoint inhibitor, an effector, an antibody or functional variant or fragment thereof, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD). In those embodiments of the method of treating, where the therapeutic agent is a checkpoint inhibitor, the checkpoint inhibitor may comprise: (i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR; (ii) one or more of atezolizumab, avelumab, cetuximab, durvalumab, ipilimumab, IPH4102, IPH43, IPH33, lirimumab, monalizumab, nivolumab, pembrolizumab, trastuzumab, pertuzumab, and their derivatives or functional equivalents; (iii) at least one of atezolizumab, nivolumab, and pembrolizumab; or the therapeutic agents comprise one or more of venetoclax, azacitidine, and pomalidomide. In those embodiments of the method of treating where the therapeutic agent is an antibody or functional variant or fragment thereof, the antibody or functional variant or fragment thereof may comprise: (a) an anti-CD20, an anti-HER2, an anti-CD52, an anti-EGFR, an anti-CD123, an anti-GD2, an anti-PDL1, and/or an anti-CD38 antibody; or (b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or (c) daratumumab, and wherein the derivative effector cell comprises a CD38 knockout, and optionally an expression of CD16 or a variant thereof.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIGS. 1A-1C show that CXCR2 expression resulted in chemotaxis of effector cells toward a CXCR2 ligand in transwell migration assays. FIG. 1A shows transgene expression in CAR/CXCR2 transduced T cell populations. FIG. 1B demonstrates that CAR/CXCR2 T cells had significantly higher chemotaxis (P<000.1) to the CXCR2 ligand, CXCL8 compared to CXCR2 negative CAR-Ts. FIG. 1C shows that CXCR2⁺ effector cells demonstrated increased chemotaxis in response to irradiated tumor media relative to controls.

FIG. 2 demonstrates CXCR2 engineered iPSC-derived CAR-T cells express uniform levels of CXCR2 and high levels of CXCR3, as determined by flow cytometry.

FIGS. 3A and 3B show transwell migration assays determining chemotaxis of CAR/CXCR2. FIG. 3A shows that CXCR2 expressing CAR iTs are chemotactic to CXCL8, and that these cells remained similarly sensitive to the CXCR3 ligand CXCL9. FIG. 3B demonstrates that CXCR2⁺ iPSC-derived CAR-T cells exerted robust cytolytic targeting of tumor targets at the indicated Effector:Target ratios.

FIGS. 4A-4C show enhanced intratumoral infiltration/retention and tumor control by CXCR2⁺ CAR-iTs, as compared to CXCR2⁻ CAR-iTs, in CXCL8 rich tumors.

FIG. 5 shows CXCR2 ligand gene expression profiles in a range of solid tumor cell lines for breast, ovarian and prostate cancers.

FIG. 6 shows in vitro analysis of upregulated CXCL8 (IL8) levels in solid tumor cell line cells as indicated following radiation and chemotherapy preconditioning treatment, respectively.

FIG. 7 illustrates the experimental design for in vivo evaluation of CXCR2⁻ and CXCR2⁺ primary CAR-T cells with and without paclitaxel preconditioning.

FIGS. 8A and 8B demonstrates that CXCR2 expressing CAR-T cells synergize with chemotherapy-based preconditioning and improve CAR-T intratumoral infiltration and tumor growth control in vivo. FIG. 8A presents in vivo tumor measurements and FIG. 8B presents in vivo tumor growth inhibition, by CAR and CAR/CXCR2 T cells with and without paclitaxel preconditioning.

FIG. 9 demonstrates intratumoral CAR-T cell infiltration and tumor site effector cell retention as evaluated in vivo for effector groups with or without preconditioning by flow cytometry.

FIG. 10 shows chemokine receptor CXCR2 expression in CAR-T cells engineered with TRAC_CAR/IL7RF and CD38_hnCD16/CXCR2 as compared to CAR-T cells engineered with TRAC_CAR/IL7RF only.

FIG. 11 shows that CXCR2⁺ CAR iT cells functionally migrated in a dose-responsive manner to varying dilutions of a CXCR2 ligand, whereas sensitivities to ligands of other C-X-C chemokine receptors were unaffected.

FIG. 12A shows an experimental design for in vivo evaluation of CXCR2⁻ and CXCR2⁺ CAR-iT cells with CY/FLU-based preconditioning. FIG. 12B shows CXCL8 (IL8) levels in serum collected 48 hrs post chemo-preconditioning.

FIGS. 13A and 13B show intratumoral CAR-iT infiltration and retention demonstrated by flow cytometry evaluation in tumors collected at specified days during the study.

FIGS. 14A and 14B show tumor measurements at specified days for mice with chemotherapy preconditioning and subsequent treatment with CXCR2⁻ and CXCR2⁺ CAR-iT cells.

### DETAILED DESCRIPTION OF THE INVENTION

Genomic modification of iPSCs (induced pluripotent stem cells) can include one or more of polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. In various embodiments, the present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cells), T cell progenitor cells, NK cell progenitor cells, T cells, NKT cells, NK cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition at a functionally inert, low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refer to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, refers to stem cells that are produced in vitro, from differentiated adult, neonatal or fetal cells that have been induced or changed, i.e., reprogrammed into cells capable of differentiating into tissues of all three germ or dermal layers: mesoderm, endoderm, and ectoderm. In some embodiments, the reporgramming process uses reprogramming factors and/or small molecule chemical driven methods. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta (i.e., are not totipotent).

As used herein, the term “multipotent stem cell” refers to a cell that has the developmental potential to differentiate into cells of one or more germ layers (i.e., ectoderm, mesoderm and endoderm), but not all three. Thus, a multipotent cell can also be termed a “partially differentiated cell.” Multipotent cells are known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to the state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) the ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed state of pluripotency. Under standard pluripotent cell culture conditions such cells remain in the primed state unless the exogenous transgene expression is maintained, wherein characteristics of the ground state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells, NK lineage cells and B lineage cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells in an MHC class I-restricted manner. A T cell can be any T cell, such as a cultured T cell, e.g., a primary T cell, or a T cell from a cultured T cell line, e.g., Jurkat, SupT1, etc., or a T cell obtained from a mammal. The T cell can be a CD3⁺ cell. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (Tb T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The term “T cell” can also refer to a genetically engineered T cell, such as a T cell modified to express a T cell receptor (TCR) or a chimeric antigen receptor (CAR). A T cell or T cell like effector cell can also be differentiated from a stem cell or progenitor cell (“a derived T cell” or “a derived T cell like effector cell”, or collectively, “a derivative T lineage cell”). A derived T cell like effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”. In some embodiments, the derivative T lineage cell is an iPSC-derived T cell obtained by differentiating an iPSC, which cells are also referred to herein as “iT” cells.

“CD4⁺ T cells” refers to a subset of T cells that express CD4 on their surface and are associated with cell-mediated immune response. They are characterized by secretion profiles following stimulation, which may include secretion of cytokines such as IFN-gamma, TNF-alpha, IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). An NK cell can be any NK cell, such as a cultured NK cell, e.g., a primary NK cell, or an NK cell from a cultured or expanded NK cell or a cell-line NK cell, e.g., NK-92, or an NK cell obtained from a mammal that is healthy or with a disease condition. As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell like effector cell may be differentiated from a stem cell or progenitor cell (“a derived NK cell” or “a derived NK cell like effector cell”, or collectively, “a derivative NK lineage cell”). A derivative NK cell like effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”. In some embodiments, the derivative NK lineage cell is an iPSC-derived NK cell obtained by differentiating an iPSC, which cells are also referred to herein as “iNK” cells.

As used herein, the term “NKT cells” or “natural killer T cells” or “NKT lineage cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified by the expression of one or more of the following markers: TCR Va24-Ja18, Vb11, CD1d, CD3, CD4, CD8, aGalCer, CD161 and CD56.

The term “effector cell” generally is applied to certain cells in the immune system that carry out a specific activity in response to stimulation and/or activation, or to cells that effect a specific function upon activation. As used herein, the term “effector cell” includes, and in some contexts is interchangeable with, immune cells, “differentiated immune cells,” and primary or differentiated cells that are edited and/or modulated to carry out a specific activity in response to stimulation and/or activation. Non-limiting examples of effector cells include primary-sourced or iPSC-derived T cells, NK cells, NKT cells, B cells, macrophages, and neutrophils.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an “isolated cell” is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained by separating the desired cells, or populations thereof, from other substances or cells in the environment, or by removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as “encoding” the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. Thus, the term “vector” comprises the construct to be delivered. A vector can be a linear or a circular molecule. A vector can be integrating or non-integrating. The major types of vectors include, but are not limited to, plasmids, episomal vectors, viral vectors, cosmids, and artificial chromosomes. Viral vectors include, but are not limited to, adenovirus vectors, adeno-associated virus vectors, retrovirus vectors, lentivirus vectors, Sendai virus vectors, and the like.

As used from time to time throughout the application, the expression of “TRAC_[construct]”, with “[construct]” being a variable expression construct having components and arrangement thereof specified in a given context, means that the expression construct is inserted at the TRAC locus to knock out TCR and with the component(s) of the expression construct expressed or co-expressed under the control of the endogenous TCR promoter. As used from time to time throughout the application, the expression of “CD38_[construct]”, with “[construct]” being a variable expression construct having components and arrangement thereof specified in a given context, means that the expression construct is inserted at the CD38 locus to knock out CD38 and with the component(s) of the expression construct expressed or co-expressed, whether under control of the endogenous CD38 promoter or under an exogenous promoter in the construct.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or is non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. “Polynucleotide” also refers to both double- and single-stranded molecules.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “subunit” refers to each separate polypeptide chain of a protein complex, where each separate polypeptide chain can form a stable folded structure by itself. Many protein molecules are composed of more than one subunit, where the amino acid sequences can either be identical for each subunit, or similar, or completely different. For example, CD3 complex is composed of CD3α, CD3ε, CD3δ, CD3γ, and CD3ζ subunits, which form the CD3ε/CD3γ, CD3ε/CD3δ, and CD3ζ/CD3ζ dimers. Within a single subunit, contiguous portions of the polypeptide chain frequently fold into compact, local, semi-independent units that are called “domains”. Many protein domains may further comprise independent “structural subunits”, also called subdomains, contributing to a common function of the domain. As such, the term “subdomain” as used herein refers to a protein domain inside of a larger domain, for example, a binding domain within an ectodomain of a cell surface receptor; or a stimulatory domain or a signaling domain of an endodomain of a cell surface receptor.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK cells or derivative T cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSCs using genomic editing. In the aspect of a source cell obtained from a specifically selected donor, disease or treatment context, the genetic imprint contributing to preferential therapeutic attributes may include any context-specific genetic or epigenetic modifications which manifest a retainable phenotype, i.e., a preferential therapeutic attribute, that is passed on to derivative cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; and/or resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. For example, synthetic effector cells, or synthetic NK cells or synthetic T cells, as used throughout this application are cells differentiated from a genomically modified iPSC, as compared to their primary counterpart obtained from natural/native sources such as peripheral blood, umbilical cord blood, or other donor tissues. In some embodiments, a synthetic cell possesses one or more non-native cell functions when compared to its closest counterpart primary cell.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof, resistance to tumor microenvironment; induction of bystander immune cells and immune modulations; improved on-target specificity with reduced off-tumor effect; and/or resistance to treatment such as chemotherapy.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil) and a tumor cell; and activating the immune cell. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, or a neutrophil. In some embodiments, the surface triggering receptor facilitates bi- or multi-specific antibody engagement between the effector cells and a specific target cell (e.g., a tumor cell) independent of the effector cells' natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some cases, and to kill two or more types of tumors in other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch protein could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing, curative or palliative properties against a disease and may be administered to ameliorate, relieve, alleviate, reverse or lessen the severity of a disease. A pharmaceutically active protein also has prophylactic properties and is used to prevent the onset of a disease or to lessen the severity of such disease or pathological condition when it does emerge. “Pharmaceutically active proteins” include an entire protein or peptide or pharmaceutically active fragments thereof. The term also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, cellular signal transduction. “Signal transduction” refers to the transmission of a molecular signal in the form of chemical modification by recruitment of protein complexes along a pathway that ultimately triggers a biochemical event in the cell. Examples of signal transduction pathways are known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes, but is not limited to, i) antigen specificity as it relates to a unique chimeric antigen receptor (CAR) or T cell receptor (TCR), ii) engager specificity as it relates to monoclonal antibodies or bispecific engagers, iii) targeting of transformed cells, iv) targeting of cancer stem cells, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that relates to the transfusion of autologous or allogeneic lymphocytes, whether the immune cells are isolated from a human donor, or effector cells obtained from in vitro differentiation of a pluripotent cell; whether they are genetically modified or not; or whether they are primary donor cells or cells that have been passaged, expanded, or immortalized, ex vivo, after isolation from a donor.

As used herein, “radiation” refers to the emission or transmission of energy in the form of waves or particles. Exemplary forms of radiation include, but are not limited to, electromagnetic radiation (e.g., radio waves, microwaves, infrared, visible light, ultraviolet, x-rays, and gamma radiation), particle radiation (e.g., alpha radiation, beta radiation, proton radiation and neutron radiation), and acoustic radiation (e.g., ultrasound, sound and seismic waves). In various embodiments, the amount of radiation is measured as a Gray (Gy), which is defined as the absorption of one joule of radiation energy per kilogram of matter. In radiation therapy, the amount of radiation applied varies depending on the type and stage of cancer being treated. For curative cases, the typical dose for a solid epithelial tumor ranges from 60 to 80 Gy, while lymphomas are typically treated with 20 to 40 Gy. Preventive (adjuvant) doses are typically around 45-60 Gy in 1.8-2 Gy fractions (for, e.g., breast, head, and neck cancers). In various embodiments, radiation may be used as a sensitizing agent as disclosed herein.

As used herein, “radiation therapy” or “radiotherapy” are used interchangeably to refer to a type of cancer treatment that involves use of radiation to damage cells by destroying the genetic material that controls how cells grow and divide. While both healthy and cancerous cells are damaged by radiation therapy, the goal of radiation therapy is to destroy as few normal, healthy cells as possible. The term “radiation therapy” often refers to external beam radiation therapy, wherein high-energy beams (e.g., x-rays, gamma rays, photons, protons, neutrons, ions, and any other forms of energy applicable to such treatments) are produced by a machine outside of the subject being treated, and are aimed at a precise point on the subject's body. However, the term “radiation therapy” also includes brachytherapy, wherein seeds, ribbons, or capsules that contain or are otherwise linked to a radiation source are placed inside the subject's body in or near a tumor or cancer cell. Included in brachytherapy are low-dose rate implants, high-dose rate implants, and permanent implants. Also included in the term “radiation therapy” is systemic radiation therapy, wherein radioactive drugs (e.g., radiopharmaceuticals or radionuclides, including radiopeptides) are given to the subject orally or intravenously and collect within the subject's body at the tumor or area where cancers cells are located. Similar to antibody-drug candidates, where an antibody that binds to a tumor antigen is linked to a toxic drug, radiopharmaceuticals incorporate a radioactive compound linked to a targeting molecule (such as an antibody) that specifically binds to a tumor antigen. Examples of radioactive compounds useful in radiopharmaceuticals include, but are not limited to calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iorn-59, krypton-81m, lutetium-177, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90. In various embodiments, radiation therapy may be used as a sensitizing agent as disclosed herein.

As used herein, “lymphodepletion” and “lympho-conditioning” are used interchangeably to refer to the destruction of lymphocytes and T cells, typically prior to immunotherapy. The purpose of lympho-conditioning prior to the administration of an adoptive cell therapy is to promote homeostatic proliferation of effector cells as well as to eliminate regulatory immune cells and other competing elements of the immune system that compete for homeostatic cytokines. Thus, lympho-conditioning is typically accomplished by administering one or more chemotherapeutic agents to the subject prior to a first dose of the adoptive cell therapy. In various embodiments, lympho-conditioning precedes the first dose of the adoptive cell therapy by a few hours to a few days. Exemplary chemotherapeutic agents useful for lympho-conditioning include, but are not limited to, cyclophosphamide (CY), fludarabine (FLU), and those described below. However, a sufficient lymphodepletion through anti-CD38 mAb could provide an alternative conditioning process (e.g., for use in an iNK cell therapy in accordance with various embodiments herein), without or with minimal need of a CY/FLU-based lympho-conditioning procedure, as further described herein.

As used herein, “homing” or “trafficking” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of a cell to a target site. In some embodiments, a homing molecule is a chemokine receptor. As used herein, “chemokine receptor” refers to a cell surface molecule that binds to a chemokine. A chemokine receptor can comprise a naturally occurring or recombinant chemokine receptor or a variant thereof. Exemplary chemokine receptors include, but are not limited to, a CXC chemokine receptor (for example, CXCR1, CXCR2, CXCR3, CXCR4, CXCR5, CXCR6, or CXCR7), a CC chemokine receptor (for example, CCR1, CCR2, CCR3, CCR4, CCR5, CCR6, CCR7, CCR8, CCR9, CCR10, or CCR11), a CX3C chemokine receptor (for example, CX3CR1), an XC chemokine receptor (for example, XCR1), or a variant thereof.

A “therapeutically sufficient amount”, as used herein, includes within its meaning a non-toxic, but sufficient and/or effective amount of a particular therapeutic agent and/or pharmaceutical composition to which it is referring to provide a desired therapeutic effect. The exact amount required will vary from subject to subject, depending on factors such as the patient's general health, the patient's age and the stage and severity of the condition being treated. In particular embodiments, a “therapeutically sufficient amount” is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (i.e., ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to the differentiation cues within the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates, forming larger aggregates, which can be mechanically or enzymatically dissociated into smaller aggregates to maintain cell proliferation within the culture and increase numbers of cells. As distinct from EB culture, cells cultured within aggregates in maintenance culture media maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “EB formation.” Monolayer differentiation, among other advantages disclosed herein, avoids the need for EB formation to initiate differentiation. Because monolayer culturing does not mimic embryo development such as is the case with EB formation, differentiation towards specific lineages is deemed to be minimal as compared to all three germ layer differentiation in EB formation.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder or stromal cells and is also not pre-conditioned by the cultivation of feeder cells.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level—successful removal, addition, or alteration of a cell function/characteristic via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA class I deficient, HLA class II deficient, or both, refers to cells that either lack, or no longer maintain, or have a reduced level of surface expression of a complete MHC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to an HLA deficient iPSC that is further modified by introducing genes expressing proteins related, but not limited to improved differentiation potential, antigen targeting, antigen presentation, antibody recognition, persistence, immune evasion, resistance to suppression, proliferation, costimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 4-1BB, DAP10, DAP12, CD24, CD3ζ, 4-1BBL, CD47, CD113, and PDL1. The cells that are “modified HLA deficient” also include cells other than iPSCs.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

The term “antibody” is used herein in the broadest sense and refers generally to an immune-response generating molecule that contains at least one binding site that specifically binds to a target, wherein the target may be an antigen, or a receptor that is capable of interacting with certain antibodies. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term “antibody” includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcaR) and those that bind IgE are called Fc-epsilon receptors (FcR). The classes of FcRs are also distinguished by the cells that express them (macrophages, granulocytes, natural killer cells, T and B cells) and the signaling properties of each receptor. Fc-gamma receptors (FcγR) include several members, FcγRI (CD64), FcγRIIA (CD32), FcγRIIB (CD32), FcγRIIIA (CD16a), and FcγRIIIB (CD16b), which differ in their antibody affinities due to their different molecular structures.

“Chimeric Receptor” is a general term used to describe an engineered, artificial, or a hybrid receptor protein molecule that is made to comprise two or more portions of amino acid sequences that are originated from at least two different proteins. The chimeric receptor proteins have been engineered to give a cell the ability to initiate signal transduction and carry out downstream function upon binding of an agonistic ligand to the receptor. Exemplary “chimeric receptors” include, but are not limited to, chimeric antigen receptors (CARs), chimeric fusion receptors (CFRs), chimeric Fc receptors (CFcRs), as well as fusions of two or more receptors.

“Chimeric Fc Receptor,” abbreviated as CFcR, is a term used to describe engineered Fc receptors having their native transmembrane and/or intracellular signaling domains modified or replaced with non-native transmembrane and/or intracellular signaling domains. In some embodiments of the chimeric Fc receptor, in addition to having one of, or both of, the transmembrane and signaling domains being non-native, one or more stimulatory domains can be introduced to the intracellular portion of the engineered Fc receptor to enhance cell activation, expansion and function upon triggering of the receptor. Unlike a chimeric antigen receptor (CAR), which contains an antigen binding domain to a target antigen, the chimeric Fc receptor binds to an Fc fragment, or the Fc region of an antibody, or the Fc region comprised in an engager or a binding molecule and activates the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fcγ receptor, by replacing its transmembrane domain and/or intracellular domain. To further improve the binding affinity of the CD16-based CFcR, the extracellular domain of CD64 or the high-affinity variants of CD16 (F176V, for example) can be incorporated. In some embodiments of the CFcR where a high affinity CD16 extracellular domain is involved, the proteolytic cleavage site comprising a serine at position 197 is eliminated or is replaced such at the extracellular domain of the receptor is non-cleavable, i.e., not subject to shedding, thereby obtaining a hnCD16-based CFcR.

CD16, a FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

I. Cells and Compositions Useful for Adoptive Cell Therapies with Enhanced Properties

Provided herein is a strategy to systematically engineer the regulatory circuitry of a clonal iPSC without impacting the differentiation potency and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic edits still have the capacity to enter cell development, and/or to mature and generate functional differentiated cells while retaining modified activities and/or properties. Unanticipated failures during directed cell differentiation from iPSCs have been attributed to aspects including, but not limited to, development stage specific gene expression or lack thereof, requirements for HLA complex presentation, protein shedding of introduced surface expressing modalities, and the need for reconfiguration of differentiation protocols enabling phenotypic and/or functional change in the cell. The present application shows that one or more selected genomic modifications of various embodiments provided herein do not negatively impact iPSC differentiation potency, and the functional effector cells derived from the engineered iPSC have enhanced and/or acquired therapeutic properties attributable to the individual or combined genomic modifications retained in the effector cells following the iPSC differentiation.

Also provided here is a solution for increasing chemokine secretion or expression on a tumor cell surface through contact with one or more sensitizing agents, such as chemotherapeutic agents, radiation and/or radioactive molecules, to improve on-target specificity with reduced off-tumor effect and/or improve homing, persistence, and cytotoxicity in the off-the-shelf adoptive cell therapy setting using effector cells derived from engineered iPSCs. In addition, the present application provides further genomic engineering aspects to achieve enhanced functionality of the effector cells, as detailed herein.

### 1. CXCR2 Overexpression

CXC chemokine receptor 2 (CXCR2), also known as CD128, interleukin 8 receptor beta (IL8Rβ), or L8 receptor type B, is a chemokine receptor mostly expressed by neutrophils, mast cells, monocytes, and macrophages. It is known that CD56 dim NK cells express CXCR2, however its expression can be downregulated upon NK cell activation. T cells typically do not express CXCR2. iPSCs and iPSC derived T cells do not express CXCR2 without transducing exogenous polynucleotides encoding CXCR2 as disclosed in this application. The chemokine IL8 (also known as CXCL8) is secreted by mononuclear macrophages, neutrophils, eosinophils, T lymphocytes, epithelial cells, and fibroblasts, and functions as a chemotactic factor by guiding the neutrophils to the site of infection. CXCL8 is also secreted by tumor cells and promotes tumor migration, invasion, angiogenesis and metastasis. CXCL8 is one of the ligands to multiple CXC chemokine receptors including CXCR1 and CXCR2. Additional chemokines known to bind to CXCR2 include, but are not limited to, CXCL1, GROβ (CXCL2), CXCL3, CXCL5, CXCL6, and CXCL7.

In various embodiments, the present application provides effector cells or iPSCs genetically engineered to comprise, among other editing as contemplated and described herein, CXCR2 or a variant thereof. A non-limiting example of the amino acid sequence of human CXCR2 is one registered as UniProtKB No: P25025. In one embodiment, the CXCR2 comprises an amino acid sequence of at least 75%, 80%, 85%, 90%, 95% or 99% identity to SEQ ID NO: 1. In some embodiments, the CXCR2 comprises an amino acid sequence of at least 90% identity to SEQ ID NO: 1. In some embodiments, the CXCR2 comprises an amino acid sequence of at least 95% identity to SEQ ID NO: 1. In some embodiments, the CXCR2 comprises the amino acid sequence of SEQ ID NO: 1. In some embodiments, the variant of CXCR2 comprises a CXCR2 isoform represented by SEQ ID NOs: 2, 3, 4, 5, or 6. In some embodiments, the variant of CXCR2 comprises an amino acid sequence of at least 75%, 80%, 85%, 90%, 95% or 99% identity to any one of SEQ ID NOs: 2, 3, 4, 5, and 6. In some embodiments, the CXCR2 is a CXCR2 variant that is effective to bind IL8. In some embodiments, the CXCR2 is a CXCR2 variant that is effective to bind IL8 and activate a signal transduction pathway in response thereto. As used herein and throughout the application, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm recognized in the art.

SEQ ID NO: 1
MEDENMESDSFEDFWKGEDLSNYSYSSTLPPELLDAAPCEPESLE
INKYFVVIIYALVELLSLLGNSLVMLVILYSRVGRSVTDVYLLNL
ALADLLFALTLPIWAASKVNGWIFGTELCKVVSLLKEVNFYSGIL
LLACISVDRYLAIVHATRTLTQKRYLVKFICLSIWGLSLLLALPV
LLFRRTVYSSNVSPACYEDMGNNTANWRMLLRILPQSFGFIVPLL
IMLFCYGFTLRTLFKAHMGQKHRAMRVIFAVVLIFLLCWLPYNLV
LLADTLMRTQVIQETCERRNHIDRALDATEILGILHSCLNPLIYA
FIGQKFRHGLLKILAIHGLISKDSLPKDSRPSFVGSSSGHTSTTL
(360 a.a. CXCR2; UniProtKB No: P25025)
SEQ ID NO: 2
MEDENMESDSFEDEW
(15 a.a. CXCR2 Isoform 1 (residues 1-15 of
CXCR2); UniProtKB No: Q6LCZ7)
SEQ ID NO: 3
MEDENMESDSFEDFWKGEDLSNYSYSSTLPPFLLDAAPCEPESLE
INKYFVVIIYALVELLSLLGNSLVMLVILYSRVGRSVTDVYLLNL
ALADLLFALTLPIWAASKVNGWIEGTELCKVVSLLKEVNFYSGIL
LLACISVDRYLAIVHATRTLTQKRYLVKFICLSIWGLSLLLALPV
LLERRTVYSSNVSPACYEDM
(200 a.a. CXCR2 Isoform 2 (residues 1-200 of
CXCR2); UniProtKB No: C9JW47)
SEQ ID NO: 4
MEDENMESDSFEDFWKGEDLSNYSYSSTLPPFLLDAAPCEPESLE
INKYFVVIIYALVELLSLLGNSLVMLVILYSRVGRSVTDVYLLNL
ALADLLFALTLPIWAASKVNGWIEGTFLCKVVSLLKEVNFYSGIL
(135 a.a. CXCR2 Isoform 3 (residues 1-135 of
CXCR2); UniProtKB No: C9JG19)
SEQ ID NO: 5
MEDENMESDSFEDFWKGEDLSNYSYSSTLPPELLDAAPCEPESLE
INKYFVVIIYALVELLSLLGNSLVMLVILYSRVGRSVTDVYLLNL
ALADLLFALTLPIWAASKVNGWIFGTELCKVVSLLKEVNFYSGIL
LLACISVDRYLAIVHATRTLTQKRYLVKFICLSIWGL
(172 a.a. CXCR2 Isoform 4 (residues 1-172 of
CXCR2); UniProtKB No: C9J1J7)
SEQ ID NO: 6
MEDENMESDSFEDFWKGEDLSNYSYSSTLPPFLLDAAPCEPESLE
INKYFVVIIYALVELLSLLGNSLVMLVILYSRVGRSVTDVYLLNL
ALADLLFALTLPIWAASKVNGWIFGTFLCKVVSLLKEVNFYSGIL
LLA
(138 a.a. CXCR2 Isoform 5 (residues 1-138 of
CXCR2); UniProtKB No: C9J2F9)

In various embodiments, the polynucleotide encoding CXCR2 or a variant thereof is inserted in a selected locus of a primary-sourced effector cell or an iPSC for deriving functional effector cells comprising the same genetic editing through directed differentiation. In some embodiments, the selected locus for CXCR2 insertion comprises a safe harbor locus, a gene locus intended to be disrupted or knocked out, a gene locus that provides an endogenous promoter that provides spacial and/or temporal control of the exogenous gene expression. In some embodiments, the selected locus for CXCR2 insertion comprises AAVS1, CCR5, ROSA26, collagen, HTRP, H11, GAPDH, RUNX1, B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CD71, CIS, CBL-B, SOCS2, PD1, CTLA4, LAG3, TIM3, or TIGIT. In one embodiment, the selected locus for CXCR2 insertion is the TCR locus. In one embodiment, the selected locus for CXCR2 insertion is the CD38 locus.

In some embodiments, the CXCR2 is co-expressed with one or more exogenous polynucleotides encoding a polypeptide of interest through separate expression constructs, or a single bi- or tri-cistronic expression cassete. In some embodiments, the single bi- or tri-cistronic expression cassete comprising CXCR2 and one or more exogenous polynucleotides encoding a polypeptide of interest comprises a 2A sequence, such that the CXCR2 and the additional polynucleotide(s) are in a single open reading frame (ORF). The bi-cistronic design allows coordinated expression of multiple polynucleotides both in timing and quantity, and under the same control mechanism that may be chosen to incorporate, for example, an inducible promoter for the expression of the single ORE. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I) (Donnelly, M L, et al, J. Gen. Virol, 82, 1027-101 (2001); Ryan, M D, et al., J. Gen. Virol., 72, 2727-2732 (2001)), and cardioviruses such as Theilovirus (e.g., Theiler's murine encephalomyelitis) and encephalomyocarditis viruses. The 2A peptides derived from FMDV, ERAV, PTV-I, and TaV are sometimes also referred to as “F2A”, “E2A”, “P2A”, and “T2A”, respectively. In some embodiments, the exogenous polynucleotides that could be co-expressed with CXCR2 encode one or more polypeptides comprising a CAR, a CD16 or a variant thereof, a cytokine, a cytokine receptor, a cytokine signaling complex, a chimeric fusion receptor, a chimeric Fc receptor, an engager, a checkpoint inhibitor, an Fc receptor, or an antibody or functional variant or fragment thereof. In one embodiment, the exogenous polynucleotides that are co-expressed with CXCR2 in a bicistronic cassette do not encode a CAR. In one particular embodiment, at least one exogenous polynucleotide that is co-expressed in a bicistronic cassette with CXCR2 encodes an exogenous CD16. In some embodiments, the primary-sourced or derived effector cells comprising CXCR2 or a variant thereof are T lineage cells. In some embodiments, the primary-sourced or derived effector cells comprising CXCR2 or a variant thereof are NK lineage cells.

Additionally provided in this application is a master cell bank comprising single cell sorted and expanded clonal engineered iPSCs having at least one modification or phenotype as provided herein, including but not limited to, CXCR2 or a variant thereof, wherein the cell bank provides clonal engineered iPSCs for additional engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

### 2. Chimeric Antigen Receptor (CAR) Expression

Applicable to the genetically engineered iPSC and derivative effector cells thereof may be any CAR design known in the art. CAR is a fusion protein generally including an ectodomain that comprises an antigen recognition domain, a transmembrane domain, and an endodomain. In some embodiments, the ectodomain can further include a signal peptide or leader sequence and/or a spacer. In some embodiments, the endodomain can further comprise a signaling domain, where the signaling domain originates from a cytoplasmic domain of a signal transducing protein specific to T and/or NK cell activation or functioning. In some embodiments, the antigen recognition domain can specifically bind an antigen. In some embodiments, the antigen recognition domain can specifically bind an antigen associated with a disease or pathogen. In some embodiments, the disease-associated antigen is a tumor antigen, wherein the tumor may be a liquid or a solid tumor. In some embodiments, the CAR is suitable to activate either T or NK lineage cells expressing the CAR. In some embodiments, the CAR is NK cell specific for comprising NK-specific signaling components. In certain embodiments, the NK cells are derived from iPSCs comprising the CAR. In some embodiments, the CAR is T cell specific for comprising T cell specific signaling components. In certain embodiments, the T cells are derived from an iPSC comprising the CAR, and the derivative T lineage cells may comprise T helper cells, cytotoxic T cells, memory T cells, regulatory T cells, natural killer T cells, αβ T cells, γδ T cells, or a combination thereof.

In certain embodiments, the antigen recognition domain comprises a murine antibody, a human antibody, a humanized antibody, a camel Ig, a shark heavy-chain-only antibody (VNAR), Ig NAR, a chimeric antibody, a recombinant antibody, or antibody fragment thereof. Non-limiting examples of antibody fragments include Fab, Fab′, F(ab′)2, F(ab′)3, Fv, single chain fragment variable (scFv), tandem scFv (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody. In some embodiments, the antigen recognition region of a CAR originates from the binding domain of a T cell receptor (TCR) that targets a tumor associated antigen (TAA).

In some embodiments of a CAR, the CAR targets antigens of hematological malignancies, which include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes. In some embodiments of a CAR, the CAR targets antigens of solid cancers, which include, but are not limited to, cancer of the brain, prostate, breast, lung, colon, uterus, skin, liver, bone, pancreas, ovary, testes, bladder, kidney, head, neck, stomach, cervix, rectum, larynx, and esophagus.

Non-limiting examples of antigens that may be targeted by a CAR include ADGRE2, B7H3, carbonic anhydrase IX (CAIX), CCR1, CCR4, carcinoembryonic antigen (CEA), CD3, CD5, CD7, CD8, CD10, CD19, CD20, CD22, CD30, CD33, CD34, CD38, CD41, CD44, CD44V6, CD49f, CD52, CD56, CD70, CD74, CD99, CD123, CD133, CD138, CD269 (BCMA), CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A1 (MAGE-A1), MICA/B, MR1, Mucin 1 (Muc-1), Mucin 16 (Muc-16), Mesothelin (MSLN), NKCSI, NKG2D ligands, c-Met, NYESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and various pathogen antigens known in the art. Non-limiting examples of pathogens include viruses, bacteria, fungi, parasites and protozoa capable of causing diseases.

In some embodiments, there is a spacer/hinge between the antigen recognition region and the transmembrane domain of the CAR, although in some other embodiments such spacer/hinge is not required. Exemplary spacers that may be included in a CAR are commonly known in the art, including, but not limited to, IgG4 spacers, CD28 spacers, CD8 spacers, or combinations of more than one spacer. The length of the spacers may also vary, from about 15 amino acids to about 300 amino acids or more. In this application, for ease of description, a spacer less than around 80 amino acids, for example 10-80 amino acids, is considered short; a spacer of about 80-180 amino acids is considered medium; and a spacer more than 180 amino acids is considered long. Non-limiting exemplary spacer peptides include those represented by an amino acid sequence of at least about 80%, about 85%, about 90%, about 95%, about 96%, about 97%, about 98%, or about 99% identity to any of SEQ ID NOs: 7-11.

SEQ ID NO: 7
IEVMYPPPYLDNEKSNGTIIHVKGKHLCPSPLFPGPSKP
(39 a.a.)
SEQ ID NO: 8
ESKYGPPCPPCPGGGSSGGGSGGQPREPQVYTLPPSQEEMTKNQV
SLTCLVKGFYPSDIAVEWESNGQPENNYKTTPPVLDSDGSFFL
(88 a.a.)
SEQ ID NO: 9
ESKYGPPCPPCPAPEFEGGPSVFLFPPKPKDTLMISRTPEVTCVV
VDVSQEDPEVQFNWYVDGVEVHNAKTKPREEQFQSTYRVVSVLT
(89 a.a.)
SEQ ID NO: 10
ESKYGPPCPPCPGGGSSGGGSGGQPREPQVYTLPPSQEEMTKNQV
SLTCLVKGFYPSDIAVEWESNGQPENNYKTTPPVLDSDGSFFLYS
RLTVDKSRWQEGNVFSCSVMHEALHNHYTQKSLSLSLGK
(129 a.a.)
SEQ ID NO: 11
ESKYGPPCPPCPAPEFEGGPSVFLFPPKPKDTLMISRTPEVTCVV
VDVSQEDPEVQFNWYVDGVEVHNAKTKPREEQFQSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVESCSVMHEALHNHYTQKSLSL
SLGK
(229 a.a.)

In some embodiments, the transmembrane domain of a CAR comprises a full length or at least a portion of the native or modified transmembrane region of 2B4, 4-1BB, BTLA, CD2, CD3δ, CD3ε, CD3γ, CD3ζ, CD4, CD8, CD8a, CD8b, CD16, CD27, CD28, CD28H, CD40, CD84, CD166, CS1, CTLA-4, DNAM1, DAP10, DAP12, FcERIγ, ICOS, ICAM-1, IL7, IL12, IL15, KIR2DL4, KIR2DS1, KIR2DS2, LAG3, PD1, NKp30, NKp44, NKp46, NKG2C, NKG2D, OX40, or T cell receptor polypeptide.

In some embodiments, the signaling peptide of the endodomain (or intracellular domain) comprises a full length or at least a portion of a polypeptide of 2B4, CD2, CD3ζ, CD3(1XX, CD8, CD28, CD28H, CD137 (4-1BB), CS1, DAP10, DAP12, DNAM1, FcERIγ, IL2Rγ, IL7R, IL21R, IL2Rβ (IL15Rβ), IL21, IL7, IL12, IL15, IL21, KIR2DS2, NKp30, NKp44, NKp46, NKG2C, or NKG2D. In one embodiment, the signaling peptide of a CAR comprises an amino acid sequence that has at least about 85%, about 90%, about 95%, about 96%, about 97%, about 98%, or about 99% identity to at least one ITAM (immunoreceptor tyrosine-based activation motif) of CD3ζ.

In certain embodiments, the endodomain further comprises at least one co-stimulatory signaling region. The co-stimulatory signaling region can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In one embodiment, the CAR applicable to the cells provided herein comprises a co-stimulatory domain derived from CD28, and a signaling domain comprising the native or modified ITAM1 of CD3ζ, represented by an amino acid sequence having at least about 85%, about 90%, about 95%, about 96%, about 97%, about 98%, or about 99% identity to SEQ ID NO: 12. In a further embodiment, the CAR comprising a co-stimulatory domain derived from CD28, and a native or modified ITAM1 of CD3ζ also comprises a hinge domain and trans-membrane domain derived from CD28, wherein an scFv may be connected to the trans-membrane domain through the hinge, and the CAR comprises an amino acid sequence of at least about 85%, about 90%, about 95%, about 96%, about 97%, about 98%, or about 99% identity to SEQ ID NO: 13. In some embodiments, the CAR comprises the amino acid sequence of SEQ ID NO: 13.

SEQ ID NO: 12
RSKRSRLLHSDYMNMTPRRPGPTRKHYQPYAPPRDFAAYRSRVKE
SRSADAPAYQQGONQLYNELNLGRREEYDVLDKRRGRDPEMGGKP
RRKNPQEGLENELQKDKMAEAFSEIGMKGERRRGKGHDGLFQGLS
TATKDTFDALHMQALPPR
(153 a.a. CD28 co-stim + CD3ζITAM)
SEQ ID NO: 13
IEVMYPPPYLDNEKSNGTIIHVKGKHLCPSPLFPGPSKPFWVLVV
VGGVLACYSLLVTVAFIIFWVRSKRSRLLHSDYMNMTPRRPGPTR
KHYQPYAPPRDFAAYRSRVKESRSADAPAYQQGQNQLYNELNLGR
REEYDVLDKRRGRDPEMGGKPRRKNPQEGLENELQKDKMAEAFSE
IGMKGERRRGKGHDGLFQGLSTATKDTEDALHMQALPPR
(219 a.a. CD28 hinge + CD28 TM +
CD28 co-stim + CD3ζITAM)

In another embodiment, the CAR applicable to the cells provided herein comprises a transmembrane domain derived from NKG2D, a co-stimulatory domain derived from 2B4, and a signaling domain comprising the native or modified CD3ζ, represented by an amino acid sequence of at least about 85%, about 90%, about 95%, about 96%, about 97%, about 98%, or about 99% identity to SEQ ID NO: 14. Said CAR comprising a transmembrane domain derived from NKG2D, a co-stimulatory domain derived from 2B4, and a signaling domain comprising the native or modified CD3ζ may further comprise a CD8 hinge, wherein the amino acid sequence of such a structure is of at least about 85%, about 90%, about 95%, about 96%, about 97%, about 98%, or about 99% identity to SEQ ID NO: 15. In some embodiments, the CAR comprises the amino acid sequence of SEQ ID NO: 15.

SEQ ID NO: 14
SNLFVASWIAVMIIFRIGMAVAIFCCFFFPSWRRKRKEKQSETSP
KEFLTIYEDVKDLKTRRNHEQEQTFPGGGSTIYSMIQSQSSAPTS
QEPAYTLYSLIQPSRKSGSRKRNHSPSENSTIYEVIGKSQPKAQN
PARLSRKELENFDVYSRVKESRSADAPAYKQGQNQLYNELNLGRR
EEYDVLDKRRGRDPEMGGKPRRKNPQEGLYNELQKDKMAEAYSEI
GMKGERRRGKGHDGLYQGLSTATKDTYDALHMQALPPR
(263 a.a NKG2D TM + 2B4 + CD3ζ)
SEQ ID NO: 15
TTTPAPRPPTPAPTIASQPLSLRPEACRPAAGGAVHTRGLDFACD
SNLEVASWIAVMIIFRIGMAVAIFCCFFFPSWRRKRKEKQSETSP
KEFLTIYEDVKDLKTRRNHEQEQTFPGGGSTIYSMIQSQSSAPTS
QEPAYTLYSLIQPSRKSGSRKRNHSPSENSTIYEVIGKSQPKAQN
PARLSRKELENFDVYSRVKFSRSADAPAYKQGQNQLYNELNLGRR
EEYDVLDKRRGRDPEMGGKPRRKNPQEGLYNELQKDKMAEAYSEI
GMKGERRRGKGHDGLYQGLSTATKDTYDALHMQALPPR
(308 a.a CD8 hinge + NKG2D TM + 2B4 + CD3ζ)

Non-limiting CAR strategies further include heterodimeric, conditionally activated CAR through dimerization of a pair of intracellular domains (see for example, U.S. Pat. No. 9,587,020); split CAR, where homologous recombination of antigen binding, hinge, and endodomains to generate a CAR (see for example, U.S. Pub. No. 2017/0183407); multi-chain CAR that allows non-covalent link between two transmembrane domains connected to an antigen binding domain and a signaling domain, respectively (see for example, U.S. Pub. No. 2014/0134142); CARs having bispecific antigen binding domain (see for example, U.S. Pat. No. 9,447,194), or having a pair of antigen binding domains recognizing same or different antigens or epitopes (see for example, U.S. Pat. No. 8,409,577), or a tandem CAR (see for example, Hegde et al., J Clin Invest. 2016; 126(8):3036-3052); inducible CAR (see for example, U.S. Pub. Nos. 2016/0046700, 2016/0058857, and 2017/0166877); switchable CAR (see for example, U.S. Pub. No. 2014/0219975); and any other designs known in the art.

In one particular embodiment of the immune cells, the iPSCs and their derivative cells that comprise polynucleotides encoding CXCR2 or a variant thereof and a CAR, the polynucleotides for CXCR2 or variant thereof and the CAR are not in a bicistronic expression cassette containing a 2A sequence. In various embodiments, the immune cell or the iPSC and its derivative effector cells comprising CXCR2 or a variant thereof, and one or more CARs have the CAR inserted in a TCR constant region (TRAC or TRBC), leading to TCR knockout, and optionally placing CAR expression under the control of the endogenous TCR promoter. The disruption of the constant region of TCRa or TCRO (TRAC or TRBC) produces a TCR^{neg} cell. In addition, the expression of TCR is also negative in a NK lineage effector cell that differentiated from iPSC. TCR^{neg} cells do not require HLA matching, have reduced alloreactivity, and are able to prevent GvHD (Graft versus Host Disease) when used in allogeneic adoptive cell therapies. Additional insertion sites of a CAR include, but are not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, NKG2A, NKG2D, CD25, CD38, CD44, CD58, CD54, CD56, CD69, CD71, CIS, CBL-B, SOCS2, PD1, CTLA4, LAG3, TIM3, and TIGIT. In another embodiment, the immune cell, the iPSC and its derivative NK cells comprise a CAR, where the CAR is inserted in the NKG2A locus or NKG2D locus, leading to NKG2A or NKG2D knockout, thereby placing CAR expression under the control of the endogenous NKG2A or NKG2D promoter.

As such, aspects of the present invention provide genomically engineered iPSCs and derivative cells obtained from differentiating genomically engineered iPSCs, wherein the iPSCs and the derivative cells comprise polynucleotides encoding CXCR2 or a variant thereof and optionally a CAR and/or one or more additional modified modalities provided herein. As shown in the present specification, effector cells comprising CXCR2 or a variant thereof provide enhanced targeting and destruction of cancer cells expressing or secreting a CXCR2 antigen, facilitating infiltration of immune cells to tumor site(s) and enhancing/extending anti-cancer responses. Additionally provided in this application is a master cell bank comprising single cell sorted and expanded clonal engineered iPSCs having at least one phenotype as provided herein, wherein the cell bank provides a platform for additional iPSC engineering and a renewable source for manufacturing off-the-shelf, engineered, homogeneous cell therapy products, including but not limited to derivative NK and T cells, which are well-defined and uniform in coinposition, and can be mass produced at significant scale in a cost-effective manner.

### 3. CD16 Knock-In

CD16 has been identified as two isoforms, Fc receptors FcγRIIIa (CD16a; NM_000569.6) and FcγRIIIb (CD16b; NM_000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” or “high affinity non-cleavable CD16” (abbreviated as hnCD16), as used herein, refers to various CD16 variants. The wildtype CD16 has low affinity and is subject to ectodomain shedding, a proteolytic cleavage process that regulates cell surface density of various cell surface molecules on leukocytes upon NK cell activation. F176V (also called F158V in some publications) is an exemplary CD16 polymorphic variant having high affinity; whereas an S197P variant is an example of a genetically engineered non-cleavable version of CD16. An engineered CD16 variant comprising both F176V and S197P has high affinity and is non-cleavable, which was described in greater detail in International Pub. No. WO2015/148926, the complete disclosure of which is incorporated herein by reference. In addition, a chimeric CD16 receptor with the ectodomain of CD16 essentially replaced with at least a portion of CD64 ectodomain can also achieve the desired high affinity and non-cleavable features of a CD16 receptor capable of carrying out ADCC. In some embodiments, the replacement ectodomain of a chimeric CD16 comprises one or more of EC1, EC2, and EC3 exons of CD64 (UniPRotKB_P12314 or its isoform or polymorphic variant).

As such, various embodiments of an exogenous CD16 introduced to a cell include functional CD16 variants and chimeric receptors thereof. In some embodiments, the functional CD16 variant is a high-affinity non-cleavable CD16 receptor (hnCD16). An hnCD16, in some embodiments, comprises both F176V and S197P; and in some embodiments, comprises F176V and with the cleavage region eliminated. In some other embodiments, an hnCD16 comprises a sequence having identity of at least 50%, 55%, 60%, 65%, 70%, 75%, 80%, 85%, 90%, 95%, 99%, 100%, or any percentage in-between, when compared to any of the exemplary sequences, SEQ ID NOs. 16, 17 and 18, each comprises at least a portion of CD64 ectodomain.

SEQ ID NO: 16
MWFLTTLLLWVPVDGQVDTTKAVITLQPPWVSVFQEETVTLHCEV
LHLPGSSSTQWFLNGTATQTSTPSYRITSASVNDSGEYRCQRGLS
GRSDPIQLEIHRGWLLLQVSSRVFTEGEPLALRCHAWKDKLVYNV
LYYRNGKAFKFFHWNSNLTILKTNISHNGTYHCSGMGKHRYTSAG
ISVTVKELFPAPVLNASVTSPLLEGNLVTLSCETKLLLQRPGLQL
YFSFYMGSKTLRGRNTSSEYQILTARREDSGLYWCEAATEDGNVL
KRSPELELQVLGLQLPTPVWFHYQVSFCLVMVLLFAVDTGLYFSV
KTNIRSSTRDWKDHKEKWRKDPQDK
(340 a.a. CD64 domain-based construction;
CD16TM; CD16ICD)
SEQ ID NO: 17
MWFLTTLLLWVPVDGQVDTTKAVITLQPPWVSVFQEETVTLHCEV
LHLPGSSSTQWFLNGTATQTSTPSYRITSASVNDSGEYRCQRGLS
GRSDPIQLEIHRGWLLLQVSSRVFTEGEPLALRCHAWKDKLVYNV
LYYRNGKAFKFFHWNSNLTILKTNISHNGTYHCSGMGKHRYTSAG
ISVTVKELFPAPVLNASVTSPLLEGNLVTLSCETKLLLQRPGLQL
YFSFYMGSKTLRGRNTSSEYQILTARREDSGLYWCEAATEDGNVL
KRSPELELQVLGLFFPPGYQVSFCLVMVLLFAVDTGLYFSVKTNI
RSSTRDWKDHKFKWRKDPQDK
(336 a.a. CD64 exon-based construction;
CD16TM; CD16ICD)
SEQ ID NO: 18
MWFLTTLLLWVPVDGQVDTTKAVITLQPPWVSVFQEETVTLHCEV
LHLPGSSSTQWELNGTATQTSTPSYRITSASVNDSGEYRCQRGLS
GRSDPIQLEIHRGWLLLQVSSRVFTEGEPLALRCHAWKDKLVYNV
LYYRNGKAFKFFHWNSNLTILKTNISHNGTYHCSGMGKHRYTSAG
ISVTVKELFPAPVLNASVTSPLLEGNLVTLSCETKLLLQRPGLQL
YFSFYMGSKTLRGRNTSSEYQILTARREDSGLYWCEAATEDGNVL
KRSPELELQVLGFFPPGYQVSFCLVMVLLFAVDTGLYFSVKTNIR
SSTRDWKDHKEKWRKDPQDK
(335 a.a. CD64 exon-based construction;
CD16TM; CD16ICD

Accordingly, provided herein are effector cells or iPSCs genetically engineered to comprise, among other editing as contemplated and described herein, an exogenous CD16 or a variant thereof, wherein the effector cells are cells from primary sources or derived from iPSC differentiation, or wherein the genetically engineered iPSCs are capable of differentiating into derived effector cells comprising the exogenous CD16 or a variant thereof introduced to the iPSCs. In some embodiments, the exogenous CD16 is a high-affinity non-cleavable CD16 receptor (hnCD16). In some embodiments, the exogenous CD16 comprises at least a portion of the CD64 ectodomain. In some embodiments, the exogenous CD16 is in a form of a CD16-based chimeric Fc receptor (CFcR) that comprises a transmembrane domain, a stimulatory domain and/or a signaling domain that is not derived from CD16.

In some embodiments, the primary-sourced or derived effector cells comprising the exogenous CD16 or variant thereof are NK lineage cells. In some embodiments, the primary-sourced or derived effector cells comprising the exogenous CD16 or variant thereof are T lineage cells. In some embodiments, the exogenous CD16 or functional variants thereof comprised in iPSC or effector cells has high affinity in binding to a ligand that triggers downstream signaling upon such binding. Non-limiting examples of ligands binding to the exogenous CD16 or functional variants thereof include not only ADCC antibodies or fragments thereof, but also to bi-, tri-, or multi-specific engagers or binders that recognize the CD16 or CD64 extracellular binding domains of the exognous CD16. Examples of bi-, tri-, or multi-specific engagers or binders are further described below in this application. As such, at least one of the aspects of the present application provides a derivative effector cell or a cell population thereof, preloaded with one or more pre-selected ADCC antibodies through an exogenous CD16 expressed on the derivative effector cell, in an amount sufficient for therapeutic use in a treatment of a condition, a disease, or an infection as further detailed in this application, wherein the exogenous CD16 comprises an extracellular binding domain of CD64, or of a CD16 having F176V and S197P.

In some other embodiments, an exogenous CD16 comprises a CD16-, or variants thereof, based CFcR. A chimeric Fc receptor (CFcR) is produced to comprise a non-native transmembrane domain, a non-native stimulatory domain and/or a non-native signaling domain by modifying or replacing the native CD16 transmembrane- and/or the intracellular-domain. The term “non-native” used herein means that the transmembrane, stimulatory or signaling domain are derived from a different receptor other than the receptor which provides the extracellular domain. In the illustration here, the CFcR based on CD16 or variants thereof does not have a transmembrane, stimulatory or signaling domain that is derived from CD16. In some embodiments, the exogenous CD16-based CFcR comprises a non-native transmembrane domain derived from CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA4, PD1, LAG3, 2B4, BTLA, CD16, IL7, L112, IL15, KIR2DL4, KIR2DS1, NKp30, NKp44, NKp46, NKG2C, NKG2D, or T cell receptor polypeptide. In some embodiments, the exogenous CD16-based CFcR comprises a non-native stimulatory/inhibitory domain derived from CD27, CD28, 4-1BB, OX40, ICOS, PD1, LAG3, 2B4, BTLA, DAP10, DAP12, CTLA4, or NKG2D polypeptide. In some embodiments, the exogenous CD16-based CFcR comprises a non-native signaling domain derived from CD3ζ, 2B4, DAP10, DAP12, DNAM1, CD137 (4-1BB), IL21, IL7, IL12, IL15, NKp30, NKp44, N-Kp46, NKGF2C, or NKG2D polypeptide. In one embodiment of the CD16-based CFcR, the provided chimeric Fc receptor comprises a transmembrane domain and a signaling domain both derived from one of IL7, IL12, IL15, NKp30, NKp44, NKp46, NKG2C, and NKG2D polypeptide. One particular embodiment of the CD16-based chimeric Fc receptor comprises a transmembrane domain of NKG2D, a stimulatory domain of 2B4, and a signaling domain of CD3ζ; wherein the extracellular domain of the CFcR is derived from a full length or partial sequence of the extracellular domain of CD64 or CD16, and wherein the extracellular domain of CD16 comprises F176V and S197P. Another exemplary embodiment of the CD16-based chimeric Fc receptor comprises a transmembrane domain and a signaling domain of CD3ζ; wherein the extracellular domain of the CFcR is derived from a full length or partial sequence of the extracellular domain of CD64 or CD16, wherein the extracellular domain of CD16 comprises F176V and S197P.

The various embodiments of the CD16-based chimeric Fc receptor as described above are capable of binding, with high affinity, to the Fc region of an antibody or fragment thereof; or to a bi-, tri-, or multi-specific engager or binder. Upon binding, the stimulatory and/or signaling domains of the chimeric receptor enable the activation and cytokine secretion of the effector cells, and the killing of the tumor cells targeted by the antibody, or the bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16-based chimeric Fc receptor, the CFcR could contribute to effector cells' killing ability while increasing the effector cells' proliferation and/or expansion potential. The antibody and the engager can bring tumor cells expressing the antigen and the effector cells expressing the CFcR into close proximity, which also contributes to the enhanced killing of the tumor cells. Exemplary tumor antigen for bi-, tri-, multi-specific engager or binders include, but are not limited to, B7H3, BCMA, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engagers or binders suitable for engaging effector cells expressing the CD16-based CFcR in attacking tumor cells include CD16 (or CD64)-CD30, CD16 (or CD64)-BCMA, CD16 (or CD64)-IL15-EPCAM, and CD16 (or CD64)-IL15-CD33.

Unlike the endogenous CD16 expressed by primary NK cells which gets cleaved from the cellular surface following NK cell activation, the various non-cleavable versions of CD16 in derivative NK cells avoid CD16 shedding and maintain constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in a derived NK cell also enables in vitro loading of an ADCC antibody to the NK cell through hnCD16 before administering the cell to a subject in need of a cell therapy. As provided herein, the hnCD16 may comprise F176V and S197P in some embodiments, or may comprise a full or partial ectodomain originated from CD64 as exemplified by SEQ ID NO: 16, 17 or 18, or may further comprise at least one of non-native transmembrane domain, stimulatory domain and signaling domain. As disclosed, the present application also provides a derivative NK cell or a cell population thereof, preloaded with one or more pre-selected ADCC antibodies in an amount sufficient for therapeutic use in a treatment of a condition, a disease, or an infection as further detailed in this application. In some embodiments, the preloaded CD38 antibody is daratumumab. In some embodiments, the derived NK cells comprising exogenous CD16 or a variant thereof, further comprise CXCR2 or a variant thereof, as provided herein. In some embodiments, the derived NK cells comprising CXCR2 or a variant thereof, and exogenous CD16 or a variant thereof, further comprise a CAR. In some embodiments, the derived NK cells comprising CXCR2 or a variant thereof, and exogenous CD16 or a variant thereof, are preloaded with one or more of an anti-HER2 antibody (e.g., trastuzumab, pertuzumab), an anti-EGFR antibody (e.g., cetuximab), or an anti-PDL1 antibody (e.g., avelumab).

Unlike primary NK cells, mature T cells from a primary source (i.e., natural/native sources such as peripheral blood, umbilical cord blood, or other donor tissues) do not express CD16. It was previously unexpected that iPSC comprising an expressed exogenous non-cleavable CD16 did not impair the T cell developmental biology and was able to differentiate into functional derivative T lineage cells that not only express the exogenous CD16, but also are capable of carrying out function through an acquired ADCC mechanism. This acquired ADCC in the derivative T lineage cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR (chimeric antigen receptor). When the derivative T lineage cell comprises acquired ADCC through exogenous CD16, including functional variants and CD16-based CFcR, expression, and when an antibody targets a different tumor antigen from the one targeted by the CAR, the antibody can be used to rescue CAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more CARs.

As such, the application provides a derivative T lineage cell comprising an exogenous CD16 or a variant thereof. In some embodiments, the derivative T lineage cell obtained herein comprises CXCR2 or a variant thereof and an exogenous CD16. In other embodiments, the derivative T lineage cell obtained herein comprises a CAR in addition to the exogenous CD16 and CXCR2 or variant thereof. In some embodiments, the exogenous CD16 comprised in the derivative T lineage cell is an hnCD16 comprising F176V and S197P. In some other embodiments, the hnCD16 comprised in the derivative T lineage cell comprises a full or partial ectodomain originated from CD64 as exemplified by SEQ ID NO: 16, 17 or 18; or may further comprise at least one of non-native transmembrane domain, stimulatory domain and signaling domain. As explained herein, such derivative T lineage cells have an acquired mechanism to target tumors with a monoclonal antibody meditated by ADCC to enhance the therapeutic effect of the antibody. As disclosed, the present application also provides a derivative T lineage cell, or a cell population thereof, preloaded with one or more pre-selected ADCC antibodies in an amount sufficient for therapeutic use in a treatment of a condition, a disease, or an infection as further detailed below.

As provided further, the cell or population thereof, comprising polynucleotides encoding CXCR2 or a variant thereof, and optionally a CAR, and/or one or more CFRs, and an exogenous CD16 or a variant thereof (“CD16^{exo}” in Table 1), may further comprise one or more additional engineered modalities described herein, and/or as shown in Table 1. Additionally provided in this application is a master cell bank comprising single cell sorted and expanded clonal engineered iPSCs having CXCR2 or a chemokine binding fragment thereof and at least one phenotype as provided herein, including but not limited to, an exogenous CD16 or a variant thereof, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

### 4. CD38 Knockout

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include antibody-dependent cell-mediated phagocytosis (ADCP) and/or complement-dependent cytotoxicity (CDC).

Other than being highly expressed on malignant cells, CD38 is also expressed on plasma cells as well as on NK cells, and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme, CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the calcium dependent process of cell adhesion. As a receptor, CD38 recognizes CD31 and regulates cytokine release and cytotoxicity in activated NK cells. CD38 is also reported to associate with cell surface proteins in lipid rafts, to regulate cytoplasmic Ca²⁺ flux, and to mediate signal transduction in lymphoid and myeloid cells.

In malignancy treatment, systemic use of CD38 antigen binding receptor transduced T cells have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38-specific antibody, NK cell reduction in both bone marrow and peripheral blood was observed, although other immune cell types, such as T cells and B cells, were unaffected despite their CD38 expression (Casneuf et al., Blood Advances. 2017; 1(23):2105-2114).

Without being limited by theories, the present application provides a strategy to leverage the full potential of CD38 targeted cancer treatment by overcoming CD38-specific antibody and/or CD38 antigen binding domain induced effector cell depletion or reduction through fratricide. In addition, since CD38 is upregulated on activated lymphocytes such as T or B cells, by suppressing and/or eliminating these activated lymphocytes using a CD38-specific antibody, such as daratumumab, in the recipient of allogeneic effector cells, host allorejection against these effector cells would be reduced and/or prevented, thereby increasing effector cell survival and persistency. As such, a CD38-specific antibody, a secreted CD38-specific engager or a CD38-CAR (chimeric antigen receptor) against activation of recipient T, Treg, NK, and/or B cells can be used as a replacement for lymphodepletion using chemotherapy such as Cy/Flu (cyclophosphamide/fludarabine) prior to adoptive cell transferring.

In addition, when targeting CD38⁺ T and pbNK cells using CD38⁻ effector cells in the presence of anti-CD38 antibodies or CD38 inhibitors, the depletion of CD38⁺ alloreactive cells increases the NAD⁺ (nicotinamide adenine dinucleotide, a substrate of CD38) availability and decreases NAD⁺ consumption related cell death, which, among other advantages, boosts effector cell responses in an immunosuppressive tumor microenvironment and supports cell rejuvenation in aging, degenerative or inflammatory diseases.

Moreover, in various embodiments, strategies provided herein for CD38 knockout are compatible with other components and processes contemplated herein, thereby generating an iPSC line comprising a CD38 knockout, a master cell bank comprising single cell sorted and expanded clonal CD38 negative iPSCs, and obtaining negative (CD38^{neg}) derivative effector cells through directed differentiation of the engineered iPSC line, wherein the derivative effector cells are protected against fratricide and allorejection when CD38 targeted therapeutic moieties are employed with the effector cells among other advantages including improved metabolic fitness, increased resistance to oxidative stress and inducing a protein expression program in the effector cell that enhances cell activation and effector function. In addition, anti-CD38 monoclonal antibody therapy significantly depletes a patient's activated immune system without adversely affecting the patient's hematopoietic stem cell compartment. A CD38^{neg} derivative cell has the ability to resist CD38 antibody mediated depletion, and may be effectively administered in combination with anti-CD38 antibody or CD38-CAR without the use of toxic conditioning agents and thus reduce and/or replace chemotherapy-based lymphodepletion.

In some embodiments, the CD38 knockout in an iPSC line is a bi-allelic knockout. In another embodiment, knocking out CD38 at the same time as inserting one or more transgenes including CXCR2 or a variant thereof as provided herein at a selected position in CD38 can be achieved, for example, by a CD38-targeted knock-in/knockout (CD38-KI/KO) construct. In some embodiments of the construct, the construct comprises a pair of CD38 targeting homology arms for position-selective insertion within the CD38 locus. In some embodiments, the preselected targeting site is within an exon of CD38. The CD38-KI/KO constructs provided herein allow the transgene(s) to express either under the CD38 endogenous promoter or under an exogenous promoter comprised in the construct. When two or more transgenes are to be inserted at a selected location in the CD38 locus, a linker sequence, for example, a 2A linker or IRES, is placed between any two transgenes. The 2A linker encodes a self-cleaving peptide derived from FMDV, ERAV, PTV-I, and TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. The exogenous promoter comprised in a CD38-KI/KO construct may be CAG, or other constitutive, inducible, temporal-, tissue-, or cell type-specific promoters including, but not limited to CMV, EF1α, PGK, and UBC. In one embodiment, the CXCR2 or a variant thereof is inserted in the CD38 locus to simultaneously knockout CD38 in iPSC. As such, this application provides an iPSC and derivative cells therefrom comprising CXCR2 or a variant thereof, a CAR and CD38 knockout.

In various embodiments, said iPSC is capable of directed differentiation to produce functional derivative hematopoietic cells including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages. In some embodiments, the CD38 negative effector cells are NK lineage cells derived from iPSCs. In some embodiments, the CD38 negative effector cells are T lineage cells derived from iPSCs. In some embodiments, the CD38 negative iPSC and its derivative cells comprise one or more additional genomic edits as described herein, including but not limited to, CXCR2 or a variant thereof, a CAR, an exogenous CD16 or a variant thereof, and additional modalities as provided.

Additionally provided in this application is a master cell bank comprising single cell sorted and expanded clonal engineered iPSCs having at least one phenotype as provided herein, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

### 5. HLA-I- and HLA-II-Deficiency

Multiple HLA class I and class II proteins must be matched for histocompatibility in allogeneic recipients to avoid allogeneic rejection problems. Provided herein is an iPSC cell line and its derivative cells differentiated therefrom with eliminated or substantially reduced expression of HLA class I and/or HLA class II proteins. HLA class I deficiency can be achieved by functional deletion of any region of the HLA class I locus (chromosome 6p21), or deletion or disruption of HLA class-I associated genes including, but not limited to, beta-2 microglobulin (B2M) gene, TAP1 gene, TAP2 gene and Tapasin. For example, the B2M gene encodes a common subunit essential for cell surface expression of all HLA class I heterodimers. B2M negative cells are HLA-I deficient. HLA class II deficiency can be achieved by functional deletion or disruption of HLA class II associated genes including, but not limited to, RFXANK, CIITA, RFX5 and RFXAP. CIITA is a transcriptional coactivator, functioning through activation of the transcription factor RFX5 required for class II protein expression. CIITA negative cells are HLA-II deficient. As such, this application provides an iPSC and derivative cells therefrom comprising HLA-I and/or HLA-II deficiency, for example by lacking B2M and/or CIITA expression, wherein the obtained derivative effector cells enable allogeneic cell therapies by eliminating the need for MHC (major histocompatibility complex) matching, and avoiding recognition and killing by host (allogeneic) T cells.

Furthermore, a lack of HLA class I expression leads to lysis by host NK cells. Therefore, in addition to the above-discussed approach of CD38 conditioning to remove activated CD38-expressing host NK cells, to overcome this “missing self” response, HLA-E, HLA-G or other non-classical HLA-I proteins may be optionally knocked in to avoid NK cell recognition and killing of the HLA-I deficient effector cells derived from an engineered iPSC. In one embodiment, the provided HLA-I deficient iPSC and its derivative cells further comprise HLA-G knock-in.

Alternatively, in one embodiment, the provided HLA-I deficient iPSC and its derivative cells further comprise one or both of CD58 knockout and CD54 knockout. CD58 (or LFA-3) and CD54 (or ICAM-1) are adhesion proteins initiating signal-dependent cell interactions, and facilitating cell, including immune cell, migration. It was previously shown that CD58 and/or CD54 disruption effectively reduces the susceptibility of HLA-I deficient iPSC-derived effector cells to allogeneic NK cell killing. While it was shown that CD58 knockout has a higher efficiency in reducing allogeneic NK cell activation than CD54 knockout, double knockout of both CD58 and CD54 was shown to provide the most enhanced reduction of NK cell activation. In some observations, the CD58 and CD54 double knockout is even more effective than HLA-G overexpression for HLA-I deficient cells in overcoming “missing-self” effect.

As provided herein, in some embodiments, the iPSC and its derivative cells comprising CXCR2 and optionally one or more of a CAR, exogenous CD16 or variant thereof, and CD38 knockout, said cells are HLA-I and/or HLA-II deficient. In some embodiments, said HLA-I and/or HLA-II deficient iPSC and its derivative cells are CD58 negative. In some other embodiments, the HLA-I and/or HLA-II deficient iPSC and its derivative cells are CD54 negative. In yet some other embodiments, the HLA-I and/or HLA-II deficient iPSC and its derivative cells are CD54 negative and CD58 negative. Further, in some embodiments of the iPSC and its derivative cells comprising CXCR2 or a variant thereof, and optionally a CAR, exogenous CD16 or a variant thereof, and CD38 knockout, said cells are HLA-I and/or HLA-II deficient and have an exogenous polynucleotide encoding HLA-G. In some embodiments of the iPSC and its derivative cells comprising CXCR2 or a variant thereof, and optionally a CAR, exogenous CD16 or a variant thereof, and CD38 knockout, the cells are HLA-I and/or HLA-II deficient and are CD58 negative. In some embodiments of the iPSC and its derivative cells comprising CXCR2 or a variant thereof, and optionally a CAR, exogenous CD16 or a variant thereof, and CD38 knockout, the cells are HLA-I and/or HLA-II deficient and are CD54 negative. In yet some other embodiments of the iPSC and its derivative cells comprising CXCR2 or a variant thereof, and optionally a CAR, exogenous CD16 or a variant thereof, and CD38 knockout, the cells are HLA-I and/or HLA-II deficient, and are both CD58 negative and CD54 negative.

In some embodiments, the engineering for HLA-I and/or HLA-II deficiency may be bypassed, or kept intact, by expressing an inactivation CAR targeting an upregulated surface protein in activated recipient immune cells to avoid allorejection. In some embodiments, the upregulated surface protein in the activated recipient immune cells includes, but is not limited to, CD38, CD25, CD69, CD44, 4-1BB, OX40, or CD40L. When the cell expresses such an inactivation CAR, it is preferable that the cell does not express, or has knockout of, the same surface protein targeted by CAR. In some embodiments, the inactivation CAR comprises at least one of a CD38-CAR, a CD25-CAR, a CD69-CAR, a CD44-CAR, a 4-1BB-CAR, an OX40-CAR, and a CD40L-CAR.

Additionally provided in this application is a master cell bank comprising single cell sorted and expanded clonal engineered iPSCs having at least one phenotype as provided herein, including but not limited to, HLA modification (“HLA” in Table 1: HLA-I and/or HLA-II deficiency with or without HLA-E or HLA-G knock in, or with knockout of one or both of CD58 and CD54), wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

### 6. Exogenously Introduced Cytokine Signaling Complex

By avoiding systemic high-dose administration of clinically relevant cytokines, the risk of dose-limiting toxicities due to such a practice is reduced while cytokine-mediated cell autonomy is being established. To achieve lymphocyte autonomy without the need to additionally administer soluble cytokines, a cytokine signaling complex comprising a partial or full length peptide of one or more of IL2, IL4, IL6, IL7, IL9, IL10, IL11, IL12, IL15, IL18, IL21, and/or their respective receptors may be introduced to the cell to enable cytokine signaling with or without the expression of the cytokine itself to achieve lymphocyte autonomy without administered soluble cytokines, thereby maintaining or improving cell growth, proliferation, expansion, and/or effector function with reduced risk of cytokine toxicities. In some embodiments, the introduced cytokine and/or its respective native or modified receptor for cytokine signaling (signaling complex) are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through an expression construct carried by a retrovirus, Sendai virus, an adenovirus, an episome, mini-circle, or RNAs, including mRNA.

Various construct designs for introducing a protein complex for signaling of one, two, or more cytokines including, but not limited to, IL2, IL4, IL6, IL7, IL9, IL10, IL11, IL12, IL15, IL18 and IL21, into the cell are provided herein. For example, in embodiments where the signaling complex is for IL15, the transmembrane (TM) domain can be native to the IL15 receptor or may be modified or replaced with a transmembrane domain of any other membrane bound proteins. In various embodiments, the cytokine signaling complex comprises an IL15 receptor fusion (IL15RF) comprising a full or partial length of IL15 and a full or partial length of IL15 receptor (IL15R). In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL15 as well as ensuring that IL15 is membrane-bound. In other embodiments, IL15Rα with truncated intracellular domain is fused to IL15 at the C-terminus through a linker, mimicking trans-presentation of IL15, maintaining IL15 membrane-bound, and additionally eliminating cis-presentation and/or any other potential signal transduction pathways mediated by a normal I5R through its intracellular domain. In other embodiments, IL15Rα is fused to I5 without an intracellular domain (IL15A), as described in International Pub. Nos. WO 2019/191495 and WO 2019/126748, the entire disclosure of each of which is incorporated herein by reference.

In various embodiments, such a truncated construct comprises an amino acid sequence of at least 75%, 80%, 85%, 90%, 95% or 99% identity to SEQ ID NO: 19. In one embodiment of the truncated IL15/IL15Rα, the construct does not comprise the last 4 amino acid residues (KSRQ) of SEQ ID NO: 19, and comprises an amino acid sequence of at least 75%, 80%, 85%, 90%, 95% or 99% identity to SEQ ID NO: 20. In some embodiments, the construct comprises the amino acid sequence of SEQ ID NO: 20. In some embodiments, the construct comprises the amino acid sequence of SEQ ID NO: 19.

SEQ ID NO: 19
MDWTWILFLVAAATRVHSGIHVFILGCFSAGLPKTEANWVNVISD
LKKIEDLIQSMHIDATLYTESDVHPSCKVTAMKCFLLELQVISLE
SGDASIHDTVENLIILANNSLSSNGNVTESGCKECEELEEKNIKE
FLQSFVHIVQMFINTSSGGGSGGGGSGGGGSGGGGSGGGSLQITC
PPPMSVEHADIWVKSYSLYSRERYICNSGFKRKAGTSSLTECVLN
KATNVAHWTTPSLKCIRDPALVHQRPAPPSTVTTAGVTPQPESLS
PSGKEPAASSPSSNNTAATTAAIVPGSQLMPSKSPSTGTTEISSH
ESSHGTPSQTTAKNWELTASASHQPPGVYPQGHSDTTVAISTSTV
LLCGLSAVSLLACYLKSRQ
(379 a.a.; signal and linker peptides
are underlined)
SEQ ID NO: 20
MDWTWILFLVAAATRVHSGIHVFILGCFSAGLPKTEANWVNVISD
LKKIEDLIQSMHIDATLYTESDVHPSCKVTAMKCFLLELQVISLE
SGDASIHDTVENLIILANNSLSSNGNVTESGCKECEELEEKNIKE
FLQSFVHIVQMFINTSSGGGSGGGGSGGGGSGGGGSGGGSLQITC
PPPMSVEHADIWVKSYSLYSRERYICNSGFKRKAGTSSLTECVLN
KATNVAHWTTPSLKCIRDPALVHQRPAPPSTVTTAGVTPQPESLS
PSGKEPAASSPSSNNTAATTAAIVPGSQLMPSKSPSTGTTEISSH
ESSHGTPSQTTAKNWELTASASHQPPGVYPQGHSDTTVAISTSTV
LLCGLSAVSLLACYL
(375 a.a.; signal and linker peptides
are underlined)

In yet other embodiments, the cytoplasmic domain of IL15Rα can be omitted without negatively impacting the autonomous feature of the effector cell equipped with IL15. In other embodiments, essentially the entire IL15Rα is removed except for the Sushi domain fused with IL15 at one end and a transmembrane domain on the other (mb-Sushi), optionally with a linker between the Sushi domain and the trans-membrane domain. The fused IL15/mb-Sushi is expressed at the cell surface through the transmembrane domain of any membrane bound protein. Thus, unnecessary signaling through IL15Rα, including cis-presentation, is eliminated when only the desirable trans-presentation of IL15 is retained. In some embodiments, the component comprising IL15 fused with Sushi domain comprises an amino acid sequence of at least 75%, 80%, 85%, 90%, 95% or 99% identity to SEQ ID NO: 21.

SEQ ID NO: 21
MDWTWILFLVAAATRVHSGIHVFILGCFSAGLPKTEANWVNVISD
LKKIEDLIQSMHIDATLYTESDVHPSCKVTAMKCFLLELQVISLE
SGDASIHDTVENLIILANNSLSSNGNVTESGCKECEELEEKNIKE
FLQSFVHIVQMFINTSSGGGSGGGGSGGGGSGGGGSGGGSLQITC
PPPMSVEHADIWVKSYSLYSRERYICNSGFKRKAGTSSLTECVLN
KATNVAHWTTPSLKCIR
(242 a.a.; signal and linker
peptides are underlined)

In other embodiments, a native or modified IL15Rβ is fused to IL15 at the C-terminus through a linker, enabling constitutive signaling and maintaining IL15 membrane-bound and trans-representation. In other embodiments, a native or modified common receptor γC is fused to IL15 at the C-terminus through a linker for constitutive signaling and membrane bound trans-presentation of the cytokine. The common receptor γC, also called the common gamma chain or CD132, is also known as IL2 receptor subunit gamma or IL2RG. γC is a cytokine receptor sub-unit that is common to the receptor complexes for many interleukin receptors, including, but not limited to, IL2, IL4, IL7, IL9, TL15 and IL21 receptor. In other embodiments, engineered IL15Rβ that forms a homodimer in absence of IL15 is useful for producing constitutive signaling of the cytokine.

In other various embodiments, the cytokine signaling complex comprises an IL7 receptor fusion (IL7RF) comprising a full or partial length of IL7 and a full or partial length of IL7 receptor. The transmembrane (TM) domain can be native to the IL7 receptor or may be modified or replaced with a transmembrane domain of any other membrane bound proteins. In some embodiments, a native (or wildtype) or modified IL7R may be fused to IL7 at the C-terminus through a linker (also referred to as “IL7RF” herein), enabling constitutive signaling and maintaining membrane-bound IL7. In some embodiments, such a construct comprises an amino acid sequence of at least 75%, 80%, 85%, 90%, 95% or 99% identity to SEQ ID NO: 22, with transmembrane domain, signal peptide and linker being flexible and varying in length and/or sequences. In some embodiments, the construct comprises the amino acid sequence of SEQ ID NO: 22.

SEQ ID NO: 22
MDWTWILFLVAAATRVHSDCDIEGKDGKQYESVLMVSIDQLLDSM
KEIGSNCLNNEFNFFKRHICDANKEGMFLFRAARKLRQFLKMNST
GDFDLHLLKVSEGTTILLNCTGQVKGRKPAALGEAQPTKSLEENK
SLKEQKKLNDLCFLKRLLQEIKTCWNKILMGTKEHSGGGSGGGGS
GGGGSGGGGSGGGSLQESGYAQNGDLEDAELDDYSFSCYSQLEVN
GSQHSLTCAFEDPDVNITNLEFEICGALVEVKCLNFRKLQEIYFI
ETKKELLIGKSNICVKVGEKSLTCKKIDLTTIVKPEAPFDLSVVY
REGANDFVVTFNTSHLQKKYVKVLMHDVAYRQEKDENKWTHVNLS
STKLTLLQRKLQPAAMYEIKVRSIPDHYFKGFWSEWSPSYYFRTP
EINNSSGEMDPILLTISILSFFSVALLVILACVLWKKRIKPIVWP
SLPDHKKTLEHLCKKPRKNLNVSFNPESFLDCQIHRVDDIQARDE
VEGFLQDTFPQQLEESEKQRLGGDVQSPNCPSEDVVITPESFGRD
SSLTCLAGNVSACDAPILSSSRSLDCRESGKNGPHVYQDLLLSLG
TTNSTLPPPFSLQSGILTLNPVAQGQPILTSLGSNQEEAYVTMSS
FYQNQ
(Signal peptide-IL7-linker-IL7R; transmembrane
domain (TM), signal peptide and linker can
vary in length and sequences)

In another embodiment, a native or modified common receptor γC is fused to IL7 at the C-terminus through a linker for constitutive and membrane-bound cytokine signaling complex. In addition, engineered IL7R that forms a homodimer in the absence of IL7 is useful for producing constitutive signaling of the cytokine as well.

One having ordinary skill in the art would appreciate that the signal peptide and the linker sequences above are illustrative and in no way limit their variations suitable for use as a signal peptide or linker. There are many suitable signal peptide or linker sequences known and available to those in the art. The ordinary skilled in the art understands that the signal peptide and/or linker sequences may be substituted for another sequence without altering the activity of the functional peptide led by the signal peptide or linked by the linker.

In iPSCs and derivative cells therefrom comprising both CAR and exogenous cytokine and/or cytokine receptor signaling (cytokine signaling complex or “IL⁺” in Table 1), the CAR and IL may be expressed in separate constructs, or may be co-expressed in a bi-cistronic construct comprising both CAR and IL. In some further embodiments, the signaling complex can be linked to either the 5′ or the 3′ end of a CAR expression construct through a self-cleaving 2A coding sequence. As such, an IL signaling complex (e.g., IL7 signaling complex) and CAR may be in a single open reading frame (ORF). In one embodiment, the signaling complex is comprised in CAR-2A-IL or IL-2A-CAR construct. When CAR-2A-IL or IL-2A-CAR is expressed, the self-cleaving 2A peptide allows the expressed CAR and IL to dissociate, and the dissociated IL can then be presented at the cell surface, with the transmembrane domain anchored in the cell membrane. The CAR-2A-IL or IL-2A-CAR bi-cistronic design allows for coordinated CAR and IL signaling complex expression both in timing and quantity, and under the same control mechanism that may be chosen to incorporate, for example, an inducible promoter or promoter with temporal or spatial specificity for the expression of the single ORF. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I) (Donnelly, M I L, et al, J. Gen. Virol, 82, 1027-101 (2001); Ryan, M D, et al., J. Gen. Virol., 72, 2727-2732 (2001)), and cardioviruses such as Theilovirus (e.g., Theiler's murine encephalomyelitis) and encephalomyocarditis viruses. The 2A peptides derived from FMDV, ERAV, PTV-I, and TaV are sometimes also referred to as “F2A”, “E2A”, “P2A”, and “T2A”, respectively.

The bi-cistronic CAR-2A-IL or IL-2A-CAR embodiment as disclosed herein is also contemplated for expression of any other cytokine or cytokine signaling complex provided herein, for example, IL2, IL4, IL6, IL9, IL10, IL11, IL12, IL18, and IL21. In some embodiments, the bi-cistronic CAR-2A-IL or IL-2A-CAR is for expression of one or more of IL2, IL4, IL7, IL9, IL15 and IL21.

In some embodiments, the iPSC and its derivative effector cells comprising a genotype that comprises one or more attributes, including CXCR2 or a variant thereof, and optionally a CAR, exogenous CD16 or a variant thereof, CD38 knockout, HLA-I and/or HLA-II deficiency, and IL⁺, may further comprise any one of the additional attributes in Table 1. In some embodiments, the iPSC, and its derivative effector cells comprising any one of the genotypes in Table 1 may additionally comprise disruption of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, 4-1BBL, CD4, CD8, CD47, CD113, CD131, CD137, CD80, PDL1, A_{2A}R, TCR, Fc receptor, an antibody, and surface triggering receptor for coupling with bi-, multi-specific or universal engagers.

As such, in various embodiments, the cytokines IL15 or IL7 and/or receptors thereof, may be introduced to iPSCs using one or more of the construct designs described above, and to their derivative cells upon iPSC differentiation. In addition, provided herein is an induced pluripotent cell (iPSC), a clonal iPSC, a clonal iPS cell line, or iPSC-derived cells comprising CXCR2 or a variant thereof, and optionally one or more of a CAR, exogenous CD16 or a variant thereof, CD38 knockout, HLA-I and/or HLA-II deficiency, IL⁺ and a polynucleotide encoding a cytokine signaling complex and/or one or more engineered modalities as disclosed herein, wherein the cytokine signaling complex comprises a partial or full peptide of a cell surface expressed exogenous cytokine and/or a receptor thereof, as described in this section, and wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

7. Genetically Engineered iPSC Line and Derivative Cells Provided Herein

In light of the above, the present application provides an immune cell, an iPSC, an iPS cell line cell, or a population thereof, and a derivative functional cell obtained from differentiating the iPSC, wherein each cell comprises an exogenous polynucleotide encoding CXCR2 or a variant thereof, and optionally one or more of a polynucleotide encoding a CAR, a polynucleotide encoding an exogenous CD16 or a variant thereof, CD38 knockout, HLA-I and/or HLA-II deficiency, IL⁺, a cytokine signaling complex, and/or one or more additional genetic modifications as described in the application, wherein the cell is an eukaryotic cell, an animal cell, a human cell, an induced pluripotent cell (iPSC), an iPSC derived effector cell, an immune cell, or a feeder cell. In some embodiments, the tumor cells at the tumor sites secrete or overexpress a chemokine that binds to CXCR2. In some embodiments, the secreted or overexpressed chemokine that binds to CXCR2 by the tumor cells at the tumor sites comprises IL8 (CXCL8). In some embodiments, the polynucleotide encoding CXCR2 or a variant thereof is inserted at a selected gene locus. In some embodiments, the functional derivative cells are hematopoietic cells including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T lineage cells, NKT lineage cells, NK lineage cells, B lineage cells, neutrophils, dendritic cells, and macrophages. In some embodiments, the functional derivative hematopoietic cells comprise effector cells having one or more functional features that are not present in a counterpart primary T, NK, NKT, and/or B cell.

In some embodiments of the derivative effector cells, the iPSCs and their derivative cells comprise an exogenous polynucleotide encoding a CXCR2 or a variant thereof, and a polynucleotide encoding a CAR. In some embodiments, the cells comprising CXCR2 or a variant thereof and a CAR are suitable for homing or migration of the effector cells to tumor sites for CAR targeted tumor killing.

In some embodiments of the derivative effector cells, the iPSCs and their derivative cells that comprise an exogenous polynucleotide encoding CXCR2 or a variant thereof, and a polynucleotide encoding a CAR have the CAR inserted in a TCR constant region (TRAC or TRBC), leading to TCR knockout, and optionally placing CAR expression under the control of the endogenous TCR promoter. The disruption of the constant region of TCRa or TCRP (TRAC or TRBC) produces a TCR^{neg} cell. In addition, the expression of TCR is also negative in a NK lineage effector cell that is differentiated from an iPSC. TCR^{neg} cells do not require HLA matching, have reduced alloreactivity, and are able to prevent GvHD (Graft versus Host Disease) when used in allogeneic adoptive cell therapies. Additional insertion sites of a CAR include, but are not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, NKG2A, NKG2D, CD25, CD38, CD44, CD58, CD54, CD56, CD69, CD71, CIS, CBL-B, SOCS2, PD1, CTLA4, LAG3, TIM3, and TIGIT. In another embodiment of the effector cell, the iPSC and its derivative cell (e.g., a derivative NK cell) described herein comprise an exogenous polynucleotide encoding a CAR, where the CAR is inserted in the NKG2A locus or NKG2D locus, leading to NKG2A or NKG2D knockout, thereby placing CAR expression under the control of the endogenous NKG2A or NKG2D promoter.

Further provided herein is an iPSC, an iPS cell line cell, or a clonal population thereof, and a derivative functional cell obtained from differentiating the iPSC, wherein each cell comprises an exogenous polynucleotide encoding a CXCR2 or a variant thereof, a polynucleotide encoding a CAR, and a polynucleotide encoding an exogenous CD16 or a variant thereof, wherein the iPSC is capable of directed differentiation to produce functional derivative hematopoietic cells. In some embodiments, said effector cells have improved ability to home or migrate to, and remain in, tumor sites which include solid tumors, and provide a tumor antigen dual targeting mechanism to tackle tumor antigen heterogeneity and tumor antigen escape. The dual targeting through CAR binding and CD16-mediated ADCC further increases tumor targeting precision, enhancing tumor killing and minimizing the impact of tumor antigen escape.

In some further embodiments, the iPSC, iPS cell line cell, or clonal population thereof, and/or derivative effector cells therefrom comprising an exogenous polynucleotide encoding CXCR2 or a variant thereof, a polynucleotide encoding a CAR, and a polynucleotide encoding an exogenous CD16 or a variant thereof are also CD38 negative, and said cells are suitable for a subject undergoing an adoptive cell therapy. In certain embodiments, the subject may additionally receive a tumor sensitizing procedure (e.g., administration of a sensitizing agent, such as a chemotherapeutic agent, radiation, or radiotherapeutic) to upregulate tumor cell chemokine expression including, but not limited to CXCL8 overexpression, to further enhance CXCR2-overexpressing effector cell homing, trafficking and retention, and cytotoxicity at the tumor sites. In some embodiments, said effector cells comprise T lineage cells. In some other embodiments, said effector cells comprise NK lineage cells.

Additionally provided is an iPSC comprising an exogenous polynucleotide encoding CXCR2 or a variant thereof, a polynucleotide encoding a CAR, and a polynucleotide encoding a cytokine signaling complex (IL) comprising a full or partial length of cytokine and/or a full or partial length of a cytokine receptor to enable cytokine signaling contributing to cell survival, persistence and/or expansion, wherein the iPSC line is capable of directed differentiation to produce functional derivative hematopoietic cells having improved survival, persistency, expansion, and effector cell function, as well as homing, trafficking, tumor site retention and cytotoxicity. In various embodiments, the exogenously introduced cytokine signaling(s) comprise the signaling of any one, two, or more of IL2, IL4, IL6, IL7, IL9, IL10, IL11, IL12, IL15, IL18, and IL21. In some embodiments, the introduced cytokine signaling complex is for IL15 signaling in the cell, and the cell is optionally an NK lineage cell. In some other embodiments, the introduced cytokine signaling complex is for IL7 signaling in the cell, and the cell is optionally a T lineage cell. In some embodiments, the introduced cytokine signaling complex is expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through a retrovirus, Sendai virus, an adenovirus, an episome, mini-circle, or RNAs including mRNA. In some embodiments, effector cells comprising CXCR2 or a variant thereof, a CAR, IL, and optionally one or more additional genetic modifications as provided in Table 1 and throughout the application are capable of maintaining or improving cell growth, proliferation, expansion, and/or effector function autonomously without contacting additionally supplied soluble cytokines in vitro or in vivo, as well as enhanced homing, trafficking, and retention at tumor sites, in which the tumor cells could be sensitized to synergize with the functional features provided to the effector cells through rational design and precision engineering of a primary-sourced immune cell or a clonal iPSC.

Also provided is an iPSC comprising an exogenous polynucleotide encoding CXCR2 or a variant thereof, a polynucleotide encoding a CAR, a B2M knockout and/or a CIITA knockout, and optionally, one or more of HLA-G overexpression, CD58 knockout and CD54 knockout, wherein the iPSC is capable of directed differentiation to produce functional derivative hematopoietic cells. In various embodiments, the iPSC and its derivative effector cells comprising an exogenous polynucleotide encoding CXCR2 or a variant thereof and a polynucleotide encoding a CAR are HLA-I and/or HLA-II deficient. In a further embodiment, the HLA-I and/or HLA-II deficient iPSC comprising an exogenous polynucleotide encoding CXCR2 or a variant thereof and a polynucleotide encoding a CAR, and its derivative effector cells, are also CD38 negative, and can be used with an anti-CD38 antibody to induce ADCC without causing effector cell elimination, thereby increasing the persistence and/or survival of the iPSC and its effector cell. In some embodiments, the effector cell has increased persistence and/or survival in vivo.

As such, the present application provides iPSCs and their functional derivative hematopoietic cells, which comprise any one of the following genotypes in Table 1. “IL”, as provided in Table 1, stands for cytokine signaling complex for one of IL2, IL4, IL6, IL7, IL9, IL10, IL11, IL12, IL15, IL18, and IL21, depending on which specific cytokine/receptor expression is selected. Further, “IL” also encompasses the IL15A embodiment, which is detailed above as a truncated fusion protein of IL15 and IL15Rα, but without an intracellular domain. Further, when iPSCs and their functional derivative hematopoietic cells have a genotype comprising both CAR and IL, the CAR and IL may be comprised in a bi-cistronic expression cassette comprising a 2A sequence. As comparison, in some other embodiments, CAR and IL are in separate expression cassettes comprised in iPSCs and their functional derivative hematopoietic cells. In one particular embodiment, the iPSCs and their functional derivative effector cells comprising both CAR and IL, IL is IL15, wherein the IL15 construct is comprised in an expression cassette with, or separate from, the CAR.

TABLE 1
Applicable Exemplary Genotypes of the Cells Provided:

| | | | | | | HLA-G or | | |
|---|---|---|---|---|---|---|---|---|
| | | | | | | (CD58^{-/-} | | |
| | | | | | B2M^{-/-} | w/or w/o | | |
| CXCR2 | CAR | CD16^{exo} | CD38^{-/-} | IL | CIITA^{-/-} | CD54^{-/-}) | Genotype | |
| ✓ | | | | | | | 1. | CXCR2 |
| ✓ | ✓ | | | | | | 2. | CXCR2 CAR |
| ✓ | | ✓ | | | | | 3. | CXCR2 CD16^{exo} |
| ✓ | | | ✓ | | | | 4. | CXCR2 CD38^{-/-} |
| ✓ | | | | ✓ | | | 5. | CXCR2 IL |
| ✓ | | | | | ✓ | | 6. | CXCR2 B2M^{-/-}CIITA^{-/-} |
| ✓ | | | | | ✓ | ✓ | 7. | CXCR2 B2M^{-/-}CIITA^{-/-} CD58^{-/-} |
| | | | | | | | 8. | CXCR2 B2M^{-/-}CIITA^{-/-} CD54^{-/-} |
| | | | | | | | 9. | CXCR2 B2M^{-/-}CIITA^{-/-} CD58^{-/-} CD54^{-/-} |
| | | | | | | | 10. | CXCR2 B2M^{-/-}CIITA^{-/-} HLA-G |
| ✓ | ✓ | ✓ | | | | | 11. | CXCR2 CAR CD16^{exo} |
| ✓ | ✓ | | ✓ | | | | 12. | CXCR2 CAR CD38^{-/-} |
| ✓ | ✓ | | | ✓ | | | 13. | CXCR2 CAR IL |
| ✓ | ✓ | | | | ✓ | | 14. | CXCR2 CAR B2M^{-/-}CIITA^{-/-} |
| ✓ | ✓ | | | | ✓ | ✓ | 15. | CXCR2 CAR B2M^{-/-}CIITA^{-/-} CD58^{-/-} |
| | | | | | | | 16. | CXCR2 CAR B2M^{-/-}CIITA^{-/-} CD54^{-/-} |
| | | | | | | | 17. | CXCR2 CAR B2M^{-/-}CIITA^{-/-} CD58^{-/-} |
| | | | | | | | | CD54^{-/-} |
| | | | | | | | 18. | CXCR2 CAR B2M^{-/-}CIITA^{-/-} HLA-G |
| ✓ | | ✓ | ✓ | | | | 19. | CXCR2 CD16^{exo} CD38^{-/-} |
| ✓ | | ✓ | | ✓ | | | 20. | CXCR2 CD16^{exo} IL |
| ✓ | | ✓ | | | ✓ | | 21. | CXCR2 CD16^{exo} B2M^{-/-}CIITA^{-/-} |
| ✓ | | ✓ | | | ✓ | ✓ | 22. | CXCR2 CD16^{exo} B2M^{-/-}CIITA^{-/-} CD58^{-/-} |
| | | | | | | | 23. | CXCR2 CD16^{exo} B2M^{-/-}CIITA^{-/-} CD54^{-/-} |
| | | | | | | | 24. | CXCR2 CD16^{exo} B2M^{-/-}CIITA^{-/-} CD58^{-/-} |
| | | | | | | | | CD54^{-/-} |
| | | | | | | | 25. | CXCR2 CD16^{exo} B2M^{-/-}CIITA^{-/-} HLA-G |
| ✓ | | | ✓ | ✓ | | | 26. | CXCR2 CD38^{-/-} IL |
| ✓ | | | ✓ | | ✓ | | 27. | CXCR2 CD38^{-/-} B2M^{-/-}CIITA^{-/-} |
| ✓ | | | ✓ | | ✓ | ✓ | 28. | CXCR2 CD38^{-/-} B2M^{-/-}CIITA^{-/-} CD58^{-/-} |
| | | | | | | | 29. | CXCR2 CD38^{-/-} B2M^{-/-}CIITA^{-/-} CD54^{-/-} |
| | | | | | | | 30. | CXCR2 CD38^{-/-} B2M^{-/-}CIITA^{-/-} CD58^{-/-} |
| | | | | | | | | CD54^{-/-} |
| | | | | | | | 31. | CXCR2 CD38^{-/-} B2M^{-/-}CIITA^{-/-} HLA-G |
| ✓ | | | | ✓ | ✓ | | 32. | CXCR2 IL B2M^{-/-}CIITA^{-/-} |
| ✓ | | | | ✓ | ✓ | ✓ | 33. | CXCR2 IL B2M^{-/-}CIITA^{-/-} CD58^{-/-} |
| | | | | | | | 34. | CXCR2 IL B2M^{-/-}CIITA^{-/-} CD54^{-/-} |
| | | | | | | | 35. | CXCR2 IL B2M^{-/-}CIITA^{-/-} CD58^{-/-} |
| | | | | | | | | CD54^{-/-} |
| | | | | | | | 36. | CXCR2 IL B2M^{-/-}CIITA^{-/-} HLA-G |
| ✓ | ✓ | ✓ | ✓ | | | | 37. | CXCR2 CAR CD16^{exo} CD38^{-/-} |
| ✓ | ✓ | ✓ | | ✓ | | | 38. | CXCR2 CAR CD16^{exo} IL |
| ✓ | ✓ | ✓ | | | ✓ | | 39. | CXCR2 CAR CD16^{exo} B2M^{-/-}CIITA^{-/-} |
| ✓ | ✓ | ✓ | | | ✓ | ✓ | 40. | CXCR2 CAR CD16^{exo} B2M^{-/-}CIITA^{-/-} CD58^{-/-} |
| | | | | | | | 41. | CXCR2 CAR CD16^{exo} B2M^{-/-}CIITA^{-/-} CD54^{-/-} |
| | | | | | | | 42. | CXCR2 CAR CD16^{exo} B2M^{-/-}CIITA^{-/-} CD58^{-/-} |
| | | | | | | | | CD54^{-/-} |
| | | | | | | | 43. | CXCR2 CAR CD16^{exo} B2M^{-/-}CIITA^{-/-} HLA-G |
| ✓ | ✓ | | ✓ | ✓ | | | 44. | CXCR2 CAR CD38^{-/-} IL |
| ✓ | ✓ | | ✓ | | ✓ | | 45. | CXCR2 CAR CD38^{-/-} B2M^{-/-}CIITA^{-/-} |
| ✓ | ✓ | | ✓ | | ✓ | ✓ | 46. | CXCR2 CAR CD38^{-/-} B2M^{-/-}CIITA^{-/-} |
| | | | | | | | | CD58^{-/-} |
| | | | | | | | 47. | CXCR2 CAR CD38^{-/-} B2M^{-/-}CIITA^{-/-} |
| | | | | | | | | CD54^{-/-} |
| | | | | | | | 48. | CXCR2 CAR CD38^{-/-} B2M^{-/-}CIITA^{-/-} |
| | | | | | | | | CD58^{-/-} CD54^{-/-} |
| | | | | | | | 49. | CXCR2 CAR CD38^{-/-} B2M^{-/-}CIITA^{-/-} HLA-G |
| ✓ | ✓ | | | ✓ | ✓ | | 50. | CXCR2 CAR IL B2M^{-/-}CIITA^{-/-} |
| ✓ | ✓ | | | ✓ | ✓ | ✓ | 51. | CXCR2 CAR IL B2M^{-/-}CIITA^{-/-} CD58^{-/-} |
| | | | | | | | 52. | CXCR2 CAR IL B2M^{-/-}CIITA^{-/-} CD54^{-/-} |
| | | | | | | | 53. | CXCR2 CAR IL B2M^{-/-}CIITA^{-/-} CD58^{-/-} |
| | | | | | | | | CD54^{-/-} |
| | | | | | | | 54. | CXCR2 CAR IL B2M^{-/-}CIITA^{-/-} HLA-G |
| ✓ | | ✓ | ✓ | ✓ | | | 55. | CXCR2 CD16^{exo} CD38^{-/-} IL |
| ✓ | | ✓ | ✓ | | ✓ | | 56. | CXCR2 CD16^{exo} CD38^{-/-} B2M^{-/-}CIITA^{-/-} |
| ✓ | | ✓ | ✓ | | ✓ | ✓ | 57. | CXCR2 CD16^{exo} CD38^{-/-} B2M^{-/-}CIITA^{-/-} |
| | | | | | | | | CD58^{-/-} |
| | | | | | | | 58. | CXCR2 CD16^{exo} CD38^{-/-} B2M^{-/-}CIITA^{-/-} |
| | | | | | | | | CD54^{-/-} |
| | | | | | | | 59. | CXCR2 CD16^{exo} CD38^{-/-} B2M^{-/-}CIITA^{-/-} |
| | | | | | | | | CD58^{-/-} CD54^{-/-} |
| | | | | | | | 60. | CXCR2 CD16^{exo} CD38^{-/-} |
| | | | | | | | | B2M^{-/-}CIITA^{-/-} HLA-G |
| ✓ | | ✓ | | ✓ | ✓ | | 61. | CXCR2 CD16^{exo} IL B2M^{-/-}CIITA^{-/-} |
| ✓ | | ✓ | | ✓ | ✓ | ✓ | 62. | CXCR2 CD16^{exo} IL B2M^{-/-}CIITA^{-/-} CD58^{-/-} |
| | | | | | | | 63. | CXCR2 CD16^{exo} IL B2M^{-/-}CIITA^{-/-} CD54^{-/-} |
| | | | | | | | 64. | CXCR2 CD16^{exo} IL B2M^{-/-}CIITA^{-/-} CD58^{-/-} |
| | | | | | | | | CD54^{-/-} |
| | | | | | | | 65. | CXCR2 CD16^{exo} IL B2M^{-/-}CIITA^{-/-} HLA-G |
| ✓ | | | ✓ | ✓ | ✓ | | 66. | CXCR2 CD38^{-/-} IL B2M^{-/-}CIITA^{-/-} |
| ✓ | | | ✓ | ✓ | ✓ | ✓ | 67. | CXCR2 CD38^{-/-} IL B2M^{-/-}CIITA^{-/-} |
| | | | | | | | | CD58^{-/-} |
| | | | | | | | 68. | CXCR2 CD38^{-/-} IL B2M^{-/-}CIITA^{-/-} |
| | | | | | | | | CD54^{-/-} |
| | | | | | | | 69. | CXCR2 CD38^{-/-} IL B2M^{-/-}CIITA^{-/-} |
| | | | | | | | | CD58^{-/-} CD54^{-/-} |
| | | | | | | | 70. | CXCR2 CD38^{-/-} IL B2M^{-/-}CIITA^{-/-} HLA-G |
| ✓ | ✓ | ✓ | ✓ | ✓ | | | 71. | CXCR2 CAR CD16^{exo} CD38^{-/-} IL |
| ✓ | ✓ | ✓ | ✓ | | ✓ | | 72. | CXCR2 CAR CD16^{exo} CD38^{-/-} B2M^{-/-}CIITA^{-/-} |
| ✓ | ✓ | ✓ | ✓ | | ✓ | ✓ | 73. | CXCR2 CAR CD16^{exo} CD38^{-/-} B2M^{-/-}CIITA^{-/-} |
| | | | | | | | | CD58^{-/-} |
| | | | | | | | 74. | CXCR2 CAR CD16^{exo} CD38^{-/-} B2M^{-/-}CIITA^{-/-} |
| | | | | | | | | CD54^{-/-} |
| | | | | | | | 75. | CXCR2 CAR CD16^{exo} CD38^{-/-} B2M^{-/-}CIITA^{-/-} |
| | | | | | | | | CD58^{-/-} CD54^{-/-} |
| | | | | | | | 76. | CXCR2 CAR CD16^{exo} CD38^{-/-} |
| | | | | | | | | B2M^{-/-}CIITA^{-/-} HLA-G |
| ✓ | ✓ | ✓ | | ✓ | ✓ | | 77. | CXCR2 CAR CD16^{exo} IL |
| | | | | | | | | B2M^{-/-}CIITA^{-/-} |
| ✓ | ✓ | ✓ | | ✓ | ✓ | ✓ | 78. | CXCR2 CAR CD16^{exo} IL B2M^{-/-}CIITA^{-/-} |
| | | | | | | | | CD58^{-/-} |
| | | | | | | | 79. | CXCR2 CAR CD16^{exo} IL B2M^{-/-}CIITA^{-/-} |
| | | | | | | | | CD54^{-/-} |
| | | | | | | | 80. | CXCR2 CAR CD16^{exo} IL B2M^{-/-}CIITA^{-/-} |
| | | | | | | | | CD58^{-/-} CD54^{-/-} |
| | | | | | | | 81. | CXCR2 CAR CD16^{exo} IL B2M^{-/-}CIITA^{-/-} HLA-G |
| ✓ | ✓ | | ✓ | ✓ | ✓ | | 82. | CXCR2 CAR CD38^{-/-} IL B2M^{-/-}CIITA^{-/-} |
| ✓ | ✓ | | ✓ | ✓ | ✓ | ✓ | 83. | CXCR2 CAR CD38^{-/-} IL B2M^{-/-}CIITA^{-/-} |
| | | | | | | | | CD58^{-/-} |
| | | | | | | | 84. | CXCR2 CAR CD38^{-/-} IL B2M^{-/-}CIITA^{-/-} |
| | | | | | | | | CD54^{-/-} |
| | | | | | | | 85. | CXCR2 CAR CD38^{-/-} IL B2M^{-/-}CIITA^{-/-} |
| | | | | | | | | CD58^{-/-} CD54^{-/-} |
| | | | | | | | 86. | CXCR2 CAR CD38^{-/-} IL B2M^{-/-}CIITA^{-/-} |
| | | | | | | | | HLA-G |
| ✓ | | ✓ | ✓ | ✓ | ✓ | | 87. | CXCR2 CD16^{exo} CD38^{-/-} IL B2M^{-/-}CIITA^{-/-} |
| ✓ | | ✓ | ✓ | ✓ | ✓ | ✓ | 88. | CXCR2 CD16^{exo} CD38^{-/-} IL B2M^{-/-}CIITA^{-/-} |
| | | | | | | | | CD58^{-/-} |
| | | | | | | | 89. | CXCR2 CD16^{exo} CD38^{-/-} IL |
| | | | | | | | | B2M^{-/-}CIITA^{-/-} CD54^{-/-} |
| | | | | | | | 90. | CXCR2 CD16^{exo} CD38^{-/-} IL |
| | | | | | | | | B2M^{-/-}CIITA^{-/-} CD58^{-/-} CD54^{-/-} |
| | | | | | | | 91. | CXCR2 CD16^{exo} CD38^{-/-} IL |
| | | | | | | | | B2M^{-/-}CIITA^{-/-} HLA-G |
| ✓ | ✓ | ✓ | ✓ | ✓ | ✓ | | 92. | CXCR2 CAR CD16^{exo} CD38^{-/-} IL |
| | | | | | | | | B2M^{-/-}CIITA^{-/-} |
| ✓ | ✓ | ✓ | ✓ | ✓ | ✓ | ✓ | 93. | CXCR2 CAR CD16^{exo} CD38^{-/-} IL |
| | | | | | | | | B2M^{-/-}CIITA^{-/-} CD58^{-/-} |
| | | | | | | | 94. | CXCR2 CAR CD16^{exo} CD38^{-/-} IL |
| | | | | | | | | B2M^{-/-}CIITA^{-/-} CD54^{-/-} |
| | | | | | | | 95. | CXCR2 CAR CD16^{exo} CD38^{-/-} IL |
| | | | | | | | | B2M^{-/-}CIITA^{-/-} CD58^{-/-} CD54^{-/-} |
| | | | | | | | 96. | CXCR2 CAR CD16^{exo} CD38^{-/-} IL |
| | | | | | | | | B2M^{-/-}CIITA^{-/-} HLA-G |

### 8. Additional Modifications

In some embodiments, the genetically modified modalities further comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some embodiments, the genetically modified iPSC and the derivative cells thereof comprise a genotype listed in Table 1. In some embodiments, the iPSC, and its derivative effector cells comprising any one of the genotypes in Table 1 may additionally comprise disruption of at least one of TAP1, TAP2, Tapasin, NLRC5, PD1, LAG3, TIM3, RFXANK, RFX5, RFXAP, and any gene in the chromosome 6p21 region; or introduction of at least one of HLA-E, 4-1BBL, CD3, CD4, CD8, CD47, CD113, CD131, CD137, CD80, PDL1, A_{2A}R, antigen-specific TCR, an Fc receptor, an engager, and a surface triggering receptor for coupling with bi-, multi-specific or universal engagers.

Engagers are fusion proteins consisting of two or more single-chain variable fragments (scFvs) of different antibodies, with at least one scFv that binds to an effector cell surface molecule or surface triggering receptor, and at least another to a target cell via a target cell specific surface molecule. Examples of engagers include, but are not limited to, bi-specific T cell engagers (BiTEs), bi-specific killer cell engagers (BiKEs), tri-specific killer cell engagers (TriKEs), multi-specific killer cell engagers, or universal engagers compatible with multiple immune cell types. Thus, engagers can be bi-specific or multi-specific. Such bi-specific or multi-specific engagers are capable of directing an effector cell (e.g., a T cell, a NK cell, an NKT cell, a B cell, a macrophage, and/or a neutrophil) to a tumor cell and activating the immune effector cell, and have shown great potential to maximize the benefits of CAR-T cell therapy.

In some embodiments, the engager is used in combination with a population of the effector cells described herein by concurrent or consecutive administration, wherein the effector cells comprise a surface molecule, or surface triggering receptor, that is recognized by the engager. In some other embodiments, the engager is a bi-specific antibody expressed by a derivative effector cell through genetically engineering an iPSC as described herein, and directed differentiation of the engineered iPSC. Exemplary effector cell surface molecules, or surface triggering receptors, that can be used for bi- or multi-specific engager recognition, or coupling, include, but are not limited to, CD3, CD28, CD5, CD16, NKG2D, CD64, CD32, CD89, NKG2C, and a chimeric Fc receptor as disclosed herein. As described herein, in some embodiments, the exogenous CD16 expressed on the surface of the derivative effector cells for engager recognition is a hnCD16, comprising a CD16 (containing F176V and optionally S197P) or CD64 extracellular domain, and native or non-native transmembrane, stimulatory and/or signaling domains as described herein. In some embodiments, the exogenous CD16 expressed on the surface of effector cells for engager recognition is a CD16-based chimeric Fc receptor (CFcR). In some embodiments, the CD16-based CFcR comprises a transmembrane domain of NKG2D, a stimulatory domain of 2B4, and a signaling domain of CD3ζ; wherein the extracellular domain of the exogenous CD16 is derived from a full length or partial sequence of the extracellular domain of CD64 or CD16; and wherein the extracellular domain of CD16 comprises F176V and optionally S197P.

In some embodiments, the target cell for an engager is a tumor cell. Exemplary tumor cell surface molecules for bi- or multi-specific engager recognition include, but are not limited to, B7H3, BCMA, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. In one embodiment, the bi-specific engager is a bi-specific antibody specific to CD3 and CD19 (CD3-CD19). In another embodiment, the bi-specific antibody is CD16-CD30 or CD64-CD30. In another embodiment, the bi-specific antibody is CD16-BCMA or CD64-BCMA. In still another embodiment, the bi-specific antibody is CD3-CD33.

In yet another embodiment, the bi-specific antibody further comprises a linker between the effector cell and tumor cell antigen binding domains. For example, a modified IL15 may be used as a linker for effector NK cells to facilitate effector cell expansion (called TriKE, or Tri-specific Killer Engager, in some publications). In one embodiment, the TriKE is CD16-IL15-EPCAM or CD64-IL15-EPCAM. In another embodiment, the TriKE is CD16-IL15-CD33 or CD64-IL15-CD33. In yet another embodiment, the TriKE is NKG2C-IL15-CD33 (“2C1533”). In addition to IL15, cytokines suitable for inclusion in the TriKE include, but are not limited to, IL2, IL4, IL6, IL7, IL9, IL10, IL11, IL12, IL18, and IL21.

In some embodiments, the surface triggering receptor for bi- or multi-specific engagers could be endogenous to the effector cells, sometimes depending on the cell types. In some other embodiments, one or more exogenous surface triggering receptors could be introduced to the effector cells using the methods and compositions provided herein, i.e., through additional engineering of an iPSC comprising a genotype listed in Table 1, then directing the differentiation of the iPSC to T, NK or any other effector cells comprising the same genotype and the surface triggering receptor as the source iPSC.

### 8. Antibodies for Immunotherapy

In some embodiments, in addition to the genomically engineered effector cells as provided herein, additional therapeutic agents comprising an antibody, or an antibody fragment that targets an antigen associated with a condition, a disease, or an indication may be used with these effector cells in a combinational therapy. In some embodiments, the antibody is used in combination with a population of the effector cells described herein by concurrent or consecutive administration to a subject. In other embodiments, such antibody or a fragment thereof may be expressed by the effector cells by genetically engineering an iPSC using an exogenous polynucleotide sequence encoding said antibody or fragment thereof, and directing differentiation of the engineered iPSC. In some embodiments, the effector cell expresses an exogenous CD16 variant, wherein the cytotoxicity of the effector cell is enhanced by the antibody via ADCC.

In some embodiments, the therapeutic antibody is a monoclonal antibody. In some embodiments, the therapeutic antibody is a humanized antibody, a humanized monoclonal antibody, or a chimeric antibody. In some embodiments, the therapeutic antibody, or antibody fragment, specifically binds to a viral antigen. In other embodiments, the antibody, or antibody fragment, specifically binds to a tumor antigen. In some embodiments, the tumor- or viral-specific antigen activates the administered iPSC-derived effector cells to enhance their killing ability. In some embodiments, the therapeutic antibodies suitable for combinational treatment as an additional therapeutic agent to the administered iPSC-derived effector cells include, but are not limited to, anti-CD20 antibodies (rituximab, veltuzumab, ofatumumab, ublituximab, ocaratuzumab, obinutuzumab), anti-HER2 antibodies (trastuzumab, pertuzumab), anti-CD52 antibodies (alemtuzumab), anti-EGFR antibodies (cetuximab), anti-GD2 antibodies (dinutuximab), anti-PDL1 antibodies (avelumab), anti-CD38 antibodies (daratumumab, isatuximab, MOR202), anti-CD123 antibodies (7G3, CSL362), anti-SLAMF7 antibodies (elotuzumab), anti-MICA/B antibodies (7C6, 6F11, 1C2) and their humanized or Fc modified variants or fragments or their functional equivalents and biosimilars. In some embodiments, the antibodies suitable for combinational treatment as an additional therapeutic agent to the administered iPSC-derived effector cells further include bi-specific or multi-specific antibodies that target more than one antigen or epitope on a target cell or recruit effector cells (e.g., T cells, NK cells, or macrophage cells) toward target cells while targeting the target cells. Such bi-specific or multi-specific antibodies function as engagers capable of directing an effector cell (e.g., a T cell, a NK cell, an NKT cell, a B cell, a macrophage, and/or a neutrophil) to a tumor cell and activating the immune effector cell, and have shown great potential to maximize the benefits of antibody therapy.

In some embodiments, the iPSC-derived effector cells comprise hematopoietic lineage cells comprising a genotype listed in Table 1. In some embodiments, the iPSC-derived effector cells comprise NK cells comprising a genotype listed in Table 1. In some embodiments, the iPSC-derived effector cells comprise T cells comprising a genotype listed in Table 1.

In some embodiments of a combination useful for treating liquid or solid tumors, the combination comprises iPSC-derived NK or T cells comprising at least CXCR2 or a variant thereof, a CAR, and exogenous CD16 or a variant thereof, and a therapeutic antibody as described above. In some embodiments of a combination useful for treating liquid or solid tumors, the combination comprises iPSC-derived NK or T cells comprising at least CXCR2 or a variant thereof, and optionally a CAR, exogenous CD16 or a variant thereof, and CD38 knockout; and a therapeutic antibody as described above. In some embodiments of a combination useful for treating liquid or solid tumors, the combination comprises iPSC-derived NK or T cells comprising at least CXCR2 or a variant thereof, and optionally a CAR, exogenous CD16 or a variant thereof, CD38 knockout and HLA-I and/or HLA-II deficiency; and a therapeutic antibody as described above. In some embodiments of a combination useful for treating liquid or solid tumors, the combination comprises iPSC-derived NK or T cells comprising at least CXCR2 or a variant thereof, and optionally a CAR, exogenous CD16 or a variant thereof, CD38 knockout, HLA-I and/or HLA-II deficiency, and an exogenous cell surface cytokine and/or receptor; and a therapeutic antibody as described above. In various embodiments, the exogenous CD16 is hnCD16. Without being limited by the theory, hnCD16 provides enhanced ADCC of the monoclonal antibody, whereas the CAR not only targets a specific tumor antigen but also prevents tumor antigen escape using a dual targeting strategy in combination with an monoclonal antibody targeting a different tumor antigen.

In some further embodiments, the iPSC-derived NK cells comprised in the combination with daratumumab comprise at least CXCR2 or a variant thereof, and optionally one or more of a CAR, exogenous CD16 or a variant thereof, IL7 or IL15, and the CAR targets at least one of B7H3, MICA/B, CD19, BCMA, CD20, CD22, CD123, HER2, CD52, EGFR, GD2, MSLN, VEGF-R2, PSMA and PDL1; wherein the IL7 or IL15 signaling complex is co- or separately expressed with the CAR.

### 9. Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Checkpoint inhibitors (CIs) are antagonists capable of reducing checkpoint gene expression or gene products, or deceasing activity of checkpoint molecules, thereby blocking inhibitory checkpoints, and restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern. Thus, one aspect of the present application provides a therapeutic approach to overcome CI resistance by including genomically-engineered functional iPSC-derived cells as provided herein in a combination therapy with CI. In one embodiment of the combination therapy, the iPSC-derived cells are NK cells. In another embodiment of the combination therapy, the iPSC-derived cells are T cells. In addition to exhibiting direct antitumor capacity, the derivative NK cells provided herein have been shown to resist PDL1-PD1 mediated inhibition, and to have the ability to enhance T cell migration, to recruit T cells to the tumor microenvironment, and to augment T cell activation at the tumor site. Therefore, the tumor infiltration of T cells facilitated by the functionally potent genomically engineered derivative NK cells indicate that said NK cells are capable of synergizing with T cell targeted immunotherapies, including the checkpoint inhibitors, to relieve local immunosuppression and to reduce tumor burden.

In some embodiments of the combination therapy, the checkpoint inhibitor is used in combination with a population of the effector cells described herein by concurrent or consecutive administration thereof to a subject. In some other embodiments, the checkpoint inhibitor is expressed by the effector cells by genetically engineering an iPSC using an exogenous polynucleotide sequence encoding said checkpoint inhibitor, or a fragment or variant thereof, and directing differentiation of the engineered iPSC. Some embodiments of the combination therapy with the effector cells described herein comprise at least one checkpoint inhibitor to target at least one checkpoint molecule; wherein the effector cells have a genotype listed in Table 1.

In one embodiment, the iPSC-derived effector cell for checkpoint inhibitor combination therapy comprises a CXCR2 or a variant thereof, and optionally one, two, three, four, five or more of: a CAR, exogenous CD16 expression, HLA-I and/or HLA-II deficiency, CD38 knockout, and cytokine signaling complex expression; wherein when B2M is knocked out, a polynucleotide encoding HLA-G or knockout of one or both of CD58 and CD54 is optionally included. In some embodiments, the derivative NK cell comprises any one of the genotypes listed in Table 1. In some embodiments, the above derivative effector cell additionally comprises deletion, disruption, or reduced expression of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, 4-1BBL, CD3, CD4, CD8, CD47, CD113, CD131, CD137, CD80, PDL1, A_{2A}R, CAR, Fc receptor, and surface triggering receptor for coupling with bi-, multi-specific or universal engagers.

In various embodiments, the derivative effector cell is obtained from differentiating an iPSC clonal line comprising one, two, three, four, five or more of: CXCR2 expression or expression of a variant thereof, CAR expression, exogenous CD16 expression, HLA-I and/or HLA-II deficiency, CD38 knockout, and cytokine signaling complex expression; wherein when B2M is knocked out, a polynucleotide encoding HLA-G or knockout of one or both of CD58 and CD54 is optionally introduced. In some embodiments, the above-described iPSC clonal line further comprises deletion, disruption, or reduced expression of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, 4-1BBL, CD3, CD4, CD8, CD47, CD113, CD131, CD137, CD80, PDL1, A_{2A}R, CAR, Fc receptor, and surface triggering receptor for coupling with bi-, multi-specific or universal engagers.

Suitable checkpoint inhibitors for combination therapy with the derivative NK or T cells as provided herein include, but are not limited to, antagonists of PD1 (Pdcdl, CD279), PDL-1 (CD274), TIM3 (Havcr2), TIGIT (WUCAM and Vstm3), LAG3 (CD223), CTLA4 (CD152), 2B4 (CD244), 4-1BB (CD137), 4-1BBL (CD137L), A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a single variable new antigen receptor (VNAR), a shark heavy-chain-only antibody (Ig NAR), chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR antibody), IPH43 (anti-MICA antibody), IPH33 (anti-TLR3 antibody), lirimumab (anti-KIR antibody), monalizumab (anti-NKG2A antibody), nivolumab (anti-PD1 mAb), pembrolizumab (anti-PD1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2):103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, and miR-29c.

Some embodiments of the combination therapy with the provided iPSC-derived effector cells comprise at least one checkpoint inhibitor to target at least one checkpoint molecule; wherein the iPSC-derived cells have a genotype listed in Table 1. Some other embodiments of the combination therapy with the provided derivative effector cells comprise two, three or more checkpoint inhibitors such that two, three, or more checkpoint molecules are targeted. In some embodiments of the combination therapy comprising at least one checkpoint inhibitor and the iPSC-derived cells having a genotype listed in Table 1, said checkpoint inhibitor is an antibody, or a humanized or Fc modified variant or fragment, or a functional equivalent or biosimilar thereof, and said checkpoint inhibitor is produced by the iPSC-derived cells by expressing an exogenous polynucleotide sequence encoding said antibody, or a fragment or variant thereof. In some embodiments, the exogenous polynucleotide sequence encoding the antibody, or a fragment or a variant thereof that inhibits a checkpoint is co-expressed with a CAR, either in separate constructs or in a bi-cistronic construct comprising both the CAR and the sequence encoding the antibody, or the fragment thereof. In some further embodiments, the sequence encoding the antibody or the fragment thereof can be linked to either the 5′ or the 3′ end of a CAR expression construct through a self-cleaving 2A coding sequence, illustrated as, for example, CAR-2A-CI or CI-2A-CAR. As such, the coding sequences of the checkpoint inhibitor and the CAR may be in a single open reading frame (ORF). When the checkpoint inhibitor is delivered, expressed and secreted as a payload by the derivative effector cells capable of infiltrating the tumor microenvironment (TME), it counteracts the inhibitory checkpoint molecule upon engaging the TME, allowing activation of the effector cells by activating modalities such as CAR or activating receptors. In some embodiments, the checkpoint inhibitor co-expressed with CAR inhibits at least one of the checkpoint molecules: PD-1, PDL-1, TIM-3, TIGIT, LAG-3, CTLA-4, 2B4, 4-1BB, 4-1BBL, A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/ILA-E, and inhibitory KIR. In some embodiments, the checkpoint inhibitor co-expressed with CAR in a derivative cell having a genotype listed in Table 1 is selected from a group comprising atezolizumab, avelumab, durvalumab, tremelimumab, ipilimumab, IPH4102, IPH43, IPH33, lirimumab, monalizumab, nivolumab, pembrolizumab, and their humanized, or Fc modified variants, fragments and their functional equivalents or biosimilars. In some embodiments, the checkpoint inhibitor co-expressed with CAR is atezolizumab, or its humanized, or Fc modified variants, fragments or their functional equivalents or biosimilars. In some other embodiments, the checkpoint inhibitor co-expressed with CAR is nivolumab, or its humanized, or Fc modified variants, fragments or their functional equivalents or biosimilars. In some other embodiments, the checkpoint inhibitor co-expressed with CAR is pembrolizumab, or its humanized, or Fc modified variants, fragments or their functional equivalents or biosimilars.

In some other embodiments of the combination therapy comprising the iPSC-derived cells provided herein and at least one antibody inhibiting a checkpoint molecule, said antibody is not produced by, or in, the iPSC-derived cells and is additionally administered before, with, or after the administering of the derivative cells having a genotype listed in Table 1. In some embodiments, the administering of one, two, three or more checkpoint inhibitors in a combination therapy with the provided derivative effector cells are simultaneous or sequential. In one embodiment of the combination treatment comprising derived NK cells or T cells having a genotype listed in Table 1, the checkpoint inhibitor included in the treatment is one or more of atezolizumab, avelumab, durvalumab, tremelimumab, ipilimumab, IPH4102, IPH43, IPH33, lirimumab, monalizurnab, nivolumab, pembrolizumab, and their humanized or Fc modified variants, fragments and their functional equivalents or biosimilars. In some embodiments of the combination treatment comprising derived NK cells or T cells having a genotype listed in Table 1, the checkpoint inhibitor included in the treatment is atezolizumab, or its humanized or Fc modified variant, fragment and its functional equivalent or biosimilar. In some embodiments of the combination treatment comprising derived NK cells or T cells having a genotype listed in Table 1, the checkpoint inhibitor included in the treatment is nivolumab, or its humanized or Fc modified variant, fragment or its functional equivalent or biosimilar. In some embodiments of the combination treatment comprising derived NK cells or T cells having a genotype listed in Table 1, the checkpoint inhibitor included in the treatment is pembrolizumab, or its humanized or Fc modified variant, fragment or its functional equivalent or biosimilar.

II. Methods for Targeted Genome Editing at Selected Locus in iPSCs

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. Therefore, targeted editing may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.” In some situations, the targeted integration site is intended to be within a coding region of a selected gene, and thus the targeted integration could disrupt the gene expression, resulting in simultaneous knock-in and knock-out (KI/KO) in one single editing step.

Inserting one or more transgenes at a selected position in a gene locus of interest (GOI) to knock-out the gene at the same time can be achieved. Gene loci suitable for simultaneous knock-in and knockout (KI/KO) include, but are not limited to, B2M, TAP1, TAP2, tapasin, NLRC5, CIITA, RFXANK, RFX5, RFXAP, TCR a or R constant region, NKG2A, NKG2D, CD38, CD25, CD69, CD71, CD44, CD58, CD54, CD56, CIS, CBL-B, SOCS2, PD1, CTLA4, LAG3, TIM3, and TIGIT. With respective site-specific targeting homology arms for position-selective insertion, it allows the transgene(s) to express either under an endogenous promoter at the site or under an exogenous promoter comprised in the construct. When two or more transgenes are to be inserted at a selected location in CD38 locus, a linker sequence, for example, a 2A linker or IRES, is placed between any two transgenes. The 2A linker encodes a self-cleaving peptide derived from, e.g., FMDV, ERAV, PTV-I, or TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. In various embodiments, the exogenous promoter may be CAG, or other constitutive, inducible, temporal-, tissue-, or cell type-specific promoters including, but not limited to CMV, EF1α, PGK, and UBC.

Available endonucleases capable of introducing specific and targeted DSBs include, but are not limited to, zinc-finger nucleases (ZFN), transcription activator-like effector nucleases (TALEN), RNA-guided CRISPR (Clustered Regular Interspaced Short Palindromic Repeats) systems. Additionally, homing endonuclease, and DICE (dual integrase cassette exchange) system utilizing phiC31 and Bxb1 integrases are also promising tools for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain”, it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a targeted Spol1 nuclease, a polypeptide comprising a Spol1 polypeptide having nuclease activity fused to a DNA binding domain, e.g., a zinc finger DNA binding domain, a TAL effector DNA binding domain, etc. that has specificity for a DNA sequence of interest.

Additional examples of targeted nucleases suitable for embodiments of the present invention include, but not limited to Bxb1, phiC31, R4, PhiBT1, and WP/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

Using Cas9 as an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction.

DICE-mediated insertion uses a pair of recombinases, for example, phiC31 and Bxb1, to provide unidirectional integration of an exogenous DNA that is tightly restricted to each enzymes' own small attB and attP recognition sites. Because these target att sites are not naturally present in mammalian genomes, they must be first introduced into the genome, at the desired integration site. See, for example, U.S. Pub. No. 2015/0140665, the disclosure of which is incorporated herein by reference.

One aspect of the present invention provides a construct comprising one or more exogenous polynucleotides for targeted genome integration. In one embodiment, the construct further comprises a pair of homologous arms specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Promising sites for targeted integration include, but are not limited to, safe harbor loci, or genomic safe harbor (GSH), which are intragenic or extragenic regions of the human genome that, theoretically, are able to accommodate predictable expression of newly integrated DNA without adverse effects on the host cell or organism. A useful safe harbor must permit sufficient transgene expression to yield desired levels of the vector-encoded protein or non-coding RNA. A safe harbor also must not predispose cells to malignant transformation nor alter cellular functions. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important in stem cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed.

Suitable sites for human genome editing, or specifically, targeted integration, include, but are not limited to, the adeno-associated virus site 1 (AAVS1), the chemokine (CC motif) receptor 5 (CCR5) gene locus and the human orthologue of the mouse ROSA26 locus. Additionally, the human orthologue of the mouse H11 locus may also be a suitable site for insertion using the composition and method of targeted integration disclosed herein. Further, collagen and HTRP gene loci may also be used as safe harbor for targeted integration. However, validation of each selected site has been shown to be necessary especially in stem cells for specific integration events, and optimization of insertion strategy including promoter election, exogenous gene sequence and arrangement, and construct design is often needed.

For targeted in/dels, the editing site is often comprised in an endogenous gene whose expression and/or function is intended to be disrupted. In some embodiments, the endogenous gene comprising a targeted in/del is associated with immune response regulation and modulation. In some other embodiments, the endogenous gene comprising a targeted in/del is associated with targeting modality, receptors, signaling molecules, transcription factors, drug target candidates, immune response regulation and modulation, or proteins suppressing engraftment, trafficking, homing, viability, self-renewal, persistence, and/or survival of stem cells and/or progenitor cells, and the derived cells therefrom.

As such, another aspect of the present invention provides a method of targeted integration in a selected locus including genome safe harbor or a preselected locus known or proven to be safe and well-regulated for continuous or temporal gene expression such as the B2M, TAP1, TAP2, Tapasin, TRAC, or CD38 locus as provided herein. In one embodiment, the genome safe harbor for the method of targeted integration comprises one or more desired integration site comprising AAVS1, CCR5, ROSA26, collagen, HTRP, H11, beta-2 microglobulin, CD38, GAPDH, TCR or RUNX1, or other loci meeting the criteria of a genome safe harbor. In one embodiment, the method of targeted integration in a cell comprising introducing a construct comprising one or more exogenous polynucleotides to the cell, and introducing a construct comprising a pair of homologous arm specific to a desired integration site and one or more exogenous sequence, to enable site specific homologous recombination by the cell host enzymatic machinery, wherein the desired integration site comprises AAVS1, CCR5, ROSA26, collagen, HTRP, H11, GAPDH, TCR or RUNX1, or other loci meeting the criteria of a genome safe harbor. Additional integration sites include an endogenous gene locus intended for disruption, such as reduction or knockout, which comprises B2M, TAP1, TAP2, tapasin, NLRC5, CIITA, RFXANK, RFX5, RFXAP, TCR a or R constant region, NKG2A, NKG2D, CD38, CD25, CD69, CD71, CD44, CD58, CD54, CD56, CIS, CBL-B, SOCS2, PD1, CTLA4, LAG3, TIM3, or TIGIT.

In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR a or R constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR a or R constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR a or R constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR a or R constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT.

Further, as provided herein, the above method for targeted integration in a safe harbor is used to insert any polynucleotide of interest, for example, polynucleotides encoding safety switch proteins, targeting modality, receptors, signaling molecules, transcription factors, pharmaceutically active proteins and peptides, drug target candidates, and proteins promoting engraftment, trafficking, homing, viability, self-renewal, persistence, and/or survival of stem cells and/or progenitor cells. In some other embodiments, the construct comprising one or more exogenous polynucleotides further comprises one or more marker genes. In one embodiment, the exogenous polynucleotide in a construct of the invention is a suicide gene encoding safety switch protein. Suitable suicide gene systems for induced cell death include, but not limited to Caspase 9 (or caspase 3 or 7) and AP1903; thymidine kinase (TK) and ganciclovir (GCV); cytosine deaminase (CD) and 5-fluorocytosine (5-FC). Additionally, some suicide gene systems are cell type specific, for example, the genetic modification of T lymphocytes with the B-cell molecule CD20 allows their elimination upon administration of mAb Rituximab. Further, modified EGFR containing epitope recognized by cetuximab can be used to deplete genetically engineered cells when the cells are exposed to cetuximab. As such, one aspect of the invention provides a method of targeted integration of one or more suicide genes encoding safety switch proteins selected from caspase 9 (caspase 3 or 7), thymidine kinase, cytosine deaminase, modified EGFR, and B cell CD20.

In some embodiments, one or more exogenous polynucleotides integrated by the method described herein are driven by operatively-linked exogenous promoters comprised in the construct for targeted integration. The promoters may be inducible, or constructive, and may be temporal-, tissue- or cell type-specific. Suitable constructive promoters for methods of the invention include, but not limited to, cytomegalovirus (CMV), elongation factor 1α (EF1α), phosphoglycerate kinase (PGK), hybrid CMV enhancer/chicken 3-actin (CAG) and ubiquitin C (UBC) promoters. In one embodiment, the exogenous promoter is CAG.

The exogenous polynucleotides integrated by the method described herein may be driven by endogenous promoters in the host genome, at the integration site. In one embodiment, the method described herein is used for targeted integration of one or more exogenous polynucleotides at AAVS1 locus in the genome of a cell. In one embodiment, at least one integrated polynucleotide is driven by the endogenous AAVS1 promoter. In another embodiment, the method described herein is used for targeted integration at ROSA26 locus in the genome of a cell. In one embodiment, at least one integrated polynucleotide is driven by the endogenous ROSA26 promoter. In still another embodiment, the method described herein is used for targeted integration at H11 locus in the genome of a cell. In one embodiment, at least one integrated polynucleotide is driven by the endogenous H11 promoter. In another embodiment, the method described herein is used for targeted integration at collagen locus in the genome of a cell. In one embodiment, at least one integrated polynucleotide is driven by the endogenous collagen promoter. In still another embodiment, the method described herein is used for targeted integration at HTRP locus in the genome of a cell. In one embodiment, at least one integrated polynucleotide is driven by the endogenous HTRP promoter. Theoretically, only correct insertions at the desired location would enable gene expression of an exogenous gene driven by an endogenous promoter.

In some embodiments, the one or more exogenous polynucleotides comprised in the construct for the methods of targeted integration are driven by one promoter. In some embodiments, the construct comprises one or more linker sequences between two adjacent polynucleotides driven by the same promoter to provide greater physical separation between the moieties and maximize the accessibility to enzymatic machinery. The linker peptide of the linker sequences may consist of amino acids selected to make the physical separation between the moieties (exogenous polynucleotides, and/or the protein or peptide encoded therefrom) more flexible or more rigid depending on the relevant function. The linker sequence may be cleavable by a protease or cleavable chemically to yield separate moieties. Examples of enzymatic cleavage sites in the linker include sites for cleavage by a proteolytic enzyme, such as enterokinase, Factor Xa, trypsin, collagenase, and thrombin. In some embodiments, the protease is one which is produced naturally by the host or it is exogenously introduced. Alternatively, the cleavage site in the linker may be a site capable of being cleaved upon exposure to a selected chemical, e.g., cyanogen bromide, hydroxylamine, or low pH. The optional linker sequence may serve a purpose other than the provision of a cleavage site. The linker sequence should allow effective positioning of the moiety with respect to another adjacent moiety for the moieties to function properly. The linker may also be a simple amino acid sequence of a sufficient length to prevent any steric hindrance between the moieties. In addition, the linker sequence may provide for post-translational modification including, but not limited to, e.g., phosphorylation sites, biotinylation sites, sulfation sites, γ-carboxylation sites, and the like. In some embodiments, the linker sequence is flexible so as not hold the biologically active peptide in a single undesired conformation. The linker may be predominantly comprised of amino acids with small side chains, such as glycine, alanine, and serine, to provide for flexibility. In some embodiments about 80 to 90 percent or greater of the linker sequence comprises glycine, alanine, or serine residues, particularly glycine and serine residues. In several embodiments, a G4S linker peptide separates the end-processing and endonuclease domains of the fusion protein. In other embodiments, a 2A linker sequence allows for two separate proteins to be produced from a single translation. Suitable linker sequences can be readily identified empirically. Additionally, suitable size and sequences of linker sequences also can be determined by conventional computer modeling techniques. In one embodiment, the linker sequence encodes a self-cleaving peptide. In one embodiment, the self-cleaving peptide is 2A. In some other embodiments, the linker sequence provides an Internal Ribosome Entry Sequence (IRES). In some embodiments, any two consecutive linker sequences are different.

The method of introducing into cells a construct comprising exogenous polynucleotides for targeted integration can be achieved using a method of gene transfer to cells known per se. In one embodiment, the construct comprises backbones of viral vectors such as adenovirus vectors, adeno-associated virus vectors, retrovirus vectors, lentivirus vectors, or Sendai virus vectors. In some embodiments, the plasmid vectors are used for delivering and/or expressing the exogenous polynucleotides to target cells (e.g., pAl-11, pXTI, pRc/CMV, pRc/RSV, pcDNAI/Neo) and the like. In some other embodiments, the episomal vector is used to deliver the exogenous polynucleotide to target cells. In some embodiments, recombinant adeno-associated viruses (rAAV) can be used for genetic engineering to introduce insertions, deletions or substitutions through homologous recombinations. Unlike lentiviruses, rAAVs do not integrate into the host genome. In addition, episomal rAAV vectors mediate homology-directed gene targeting at much higher rates compared to transfection of conventional targeting plasmids. In some embodiments, an AAV6 or AAV2 vector is used to introduce insertions, deletions or substitutions in a target site in the genome of iPSCs. In some embodiments, the genomically modified iPSCs and their derivative cells obtained using the methods and compositions described herein comprise at least one genotype listed in Table 1.

III. Method of Obtaining and Maintaining Genome-Engineered iPSCs

The present invention also provides a method of obtaining and maintaining genome-engineered iPSCs comprising one or more targeted edits (e.g., multiplex genomic engineering) at one or more desired sites, wherein the one or more targeted edits remain intact and functional in expanded genome-engineered iPSCs or the iPSC-derived non-pluripotent cells at the respective selected editing site. The targeted editing introduces into the genome iPSC, and derivative cells therefrom, insertions, deletions, and/or substitutions (i.e., targeted integration and/or in/dels at selected sites). In comparison to direct engineering of patient-sourced, peripheral blood originated primary effector cells, the many benefits of obtaining genomically-engineered iPSC-derived effector cells through editing and differentiating iPSC as provided herein include, but are not limited to: unlimited source for engineered effector cells; no need for repeated manipulation of the effector cells, especially when multiple engineered modalities are involved; the obtained effector cells are rejuvenated for having elongated telomere and experiencing less exhaustion; the effector cell population is homogeneous in terms of editing site, copy number, and void of allelic variation, random mutations and expression variegation, largely due to the enabled clonal selection in engineered iPSCs as provided herein.

In some embodiments, the genome-engineered iPSCs comprising one or more targeted edits at one or more selected sites are maintained, passaged and expanded as single cells for an extended period in cell maintenance culture medium (FMM), wherein the iPSCs retain the targeted editing and functional modification at the selected site(s). The iPSCs cultured in FMM have been shown to continue to maintain their undifferentiated, and ground or naïve, profile; provided genomic stability without the need for culture cleaning or selection; and are readily to give rise to all three somatic lineages, in vitro differentiation via embryoid bodies or monolayer (without formation of embryoid bodies); and in vivo differentiation by teratoma formation. See, for example, International Pub. No. WO2015/134652, the disclosure of which is incorporated herein by reference.

In some embodiments, the genome-engineered iPSCs comprising one or more targeted integrations and/or in/dels are maintained, passaged and expanded in a medium (FMM) comprising a MEK inhibitor, a GSK3 inhibitor, and a ROCK inhibitor, and free of, or essentially free of, TGFβ receptor/ALK5 inhibitors, wherein the iPSCs retain the intact and functional targeted edits at the selected sites.

Another aspect of the invention provides a method of generating genome-engineered iPSCs through targeted editing of iPSCs; or through first generating genome-engineered non-pluripotent cells by targeted editing, and then reprogramming the selected/isolated genome-engineered non-pluripotent cells to obtain iPSCs comprising the same targeted editing as the non-pluripotent cells. A further aspect of the invention provides genome-engineering non-pluripotent cells which are concurrently undergoing reprogramming by introducing targeted integration and/or targeted in/dels to the cells, wherein the contacted non-pluripotent cells are under sufficient conditions for reprogramming, and wherein the conditions for reprogramming comprise contacting non-pluripotent cells with one or more reprogramming factors and small molecules. In various embodiments of the method for concurrent genome-engineering and reprogramming, the targeted integrations and/or targeted in/dels may be introduced to the non-pluripotent cells prior to, or essentially concomitantly with, initiating reprogramming by contacting the non-pluripotent cells with one or more reprogramming factors and optionally one or more small molecules.

In some embodiments, to concurrently genome-engineer and reprogram non-pluripotent cells, the targeted integrations and/or in/dels may also be introduced to the non-pluripotent cells after the multi-day process of reprogramming is initiated by contacting the non-pluripotent cells with one or more reprogramming factors and small molecules, and wherein the vectors carrying the constructs are introduced before the reprogramming cells present stable expression of one or more endogenous pluripotent genes including but not limited to SSEA4, Tra181 and CD30.

In some embodiments, the reprogramming is initiated by contacting the non-pluripotent cells with at least one reprogramming factor, and optionally a combination of a TGFβ receptor/ALK inhibitor, a MEK inhibitor, a GSK3 inhibitor and a ROCK inhibitor. In some embodiments, the genome-engineered iPSCs produced through any methods above are further maintained and expanded using a mixture comprising a combination of a MEK inhibitor, a GSK3 inhibitor and a ROCK inhibitor.

In some embodiments of the method of generating genome-engineered iPSCs, the method comprises: genomically engineering an iPSC by introducing one or more targeted integrations and/or in/dels into iPSCs to obtain genome-engineered iPSCs having a genotype provided herein. Alternatively, the method of generating genome-engineered iPSCs comprises: (a) introducing one or more targeted edits into non-pluripotent cells to obtain genome-engineered non-pluripotent cells comprising targeted integrations and/or in/dels at selected sites, and (b) contacting the genome-engineered non-pluripotent cells with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted integrations and/or in/dels at selected sites. Alternatively, the method of generating genome-engineered iPSCs comprises: (a) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate the reprogramming of the non-pluripotent cells; (b) introducing one or more targeted integrations and/or in/dels into the reprogramming non-pluripotent cells for genome-engineering; and (c) obtaining clonal genome-engineered iPSCs comprising targeted integrations and/or in/dels at selected sites. Any of the above methods may further comprise single cell sorting of the genome-engineered iPSCs to obtain a clonal iPSC, and/or screening for off-target editing and abnormal karyotypes in the genome-engineered iPSCs. Through clonal expansion of the genome-engineered iPSCs, a master cell bank is generated to comprise single cell sorted and expanded clonal engineered iPSCs having at least one phenotype as provided herein. The master cell bank is subsequently cryopreserved, providing a platform for additional iPSC engineering and a renewable source for manufacturing off-the-shelf, engineered, homogeneous cell therapy products, which are well-defined and uniform in composition, and can be mass produced at significant scale in a cost-effective manner.

The reprogramming factors are selected from the group consisting of OCT4, SOX2, NANOG, KLF4, LIN28, C-MYC, ECAT1, UTF1, ESRRB, SV40LT, HESRG, CDH1, TDGF1, DPPA4, DNMT3B, ZIC3, L1TD1, and any combinations thereof as disclosed in International Pub. Nos. WO2015/134652 and WO 2017/066634, the disclosures of which are incorporated herein by reference. The one or more reprogramming factors may be in the form of polypeptides. The reprogramming factors may also be in the form of polynucleotides encoding the reprogramming factors, and thus may be introduced to the non-pluripotent cells by vectors such as, a retrovirus, a Sendai virus, an adenovirus, an episome, a plasmid, and a mini-circle. In particular embodiments, the one or more polynucleotides encoding at least one reprogramming factor are introduced by a lentiviral vector. In some embodiments, the one or more polynucleotides are introduced by an episomal vector. In various other embodiments, the one or more polynucleotides are introduced by a Sendai viral vector. In some embodiments, the one or more polynucleotides introduced by a combination of plasmids. See, for example, International Pub. No. WO2019/075057A1, the disclosure of which is incorporated herein by reference.

In some embodiments, the non-pluripotent cells are transfected with multiple constructs comprising different exogenous polynucleotides and/or different promoters by multiple vectors for targeted integration at the same or different selected sites. These exogenous polynucleotides may comprise a suicide gene, or a gene encoding targeting modality, receptors, signaling molecules, transcription factors, pharmaceutically active proteins and peptides, drug target candidates, or a gene encoding a protein promoting engraftment, trafficking, homing, viability, self-renewal, persistence, and/or survival of the iPSCs or derivative cells thereof. In some embodiments, the exogenous polynucleotides encode RNA, including but not limited to siRNA, shRNA, miRNA and antisense nucleic acids. These exogenous polynucleotides may be driven by one or more promoters selected form the group consisting of constitutive promoters, inducible promoters, temporal-specific promoters, and tissue or cell type specific promoters. Accordingly, the polynucleotides are expressible when under conditions that activate the promoter, for example, in the presence of an inducing agent or in a particular differentiated cell type. In some embodiments, the polynucleotides are expressed in iPSCs and/or in cells differentiated from the iPSCs. In one embodiment, one or more suicide gene is driven by a constitutive promoter, for example Capase-9 driven by CAG. These constructs comprising different exogenous polynucleotides and/or different promoters can be transfected to non-pluripotent cells either simultaneously or consecutively. The non-pluripotent cells subjected to targeted integration of multiple constructs can simultaneously contact the one or more reprogramming factors to initiate the reprogramming process concurrently with the genomic engineering, thereby obtaining genome-engineered iPSCs comprising multiple targeted integrations in the same pool of cells. As such, this robust method enables a concurrent reprogramming and engineering strategy to derive a clonal genomically-engineered iPSCs with multiple modalities integrated to one or more selected target sites.

IV. A Method of Obtaining Genetically-Engineered Effector Cells by Differentiating Genome-Engineered iPSC

A further aspect of the present invention provides a method of in vivo differentiation of genome-engineered iPSCs by teratoma formation, wherein the differentiated cells derived in vivo from the genome-engineered iPSCs retain the intact and functional targeted edits including targeted integration(s) and/or in/dels at the desired site(s). In some embodiments, the differentiated cells derived in vivo from the genome-engineered iPSCs via teratoma formation comprise one or more inducible suicide genes integrated at one or more desired sites comprising AAVS1, CCR5, ROSA26, collagen, HTRP H11, beta-2 microglobulin, CD38, GAPDH, TCR or RUNX1, or other loci meeting the criteria of a genome safe harbor. In some other embodiments, the differentiated cells derived in vivo from the genome-engineered iPSCs via teratoma formation comprise polynucleotides encoding targeting modalities, or encoding proteins promoting trafficking, homing, viability, self-renewal, persistence, and/or survival of stem cells and/or progenitor cells. In some embodiments, the differentiated cells derived in vivo from the genome-engineered iPSCs via teratoma formation comprising one or more inducible suicide genes further comprise one or more in/dels in endogenous genes associated with immune response regulation and mediation. In some embodiments, the in/del is comprised in one or more endogenous checkpoint genes. In some embodiments, the in/del is comprised in one or more endogenous T cell receptor genes. In some embodiments, the in/del is comprised in one or more endogenous MHC class I suppressor genes. In some embodiments, the in/del is comprised in one or more endogenous genes associated with the major histocompatibility complex. In some embodiments, the in/del is comprised in one or more endogenous genes including, but not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR a or R constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, and TIGIT.

In some embodiments, the genome-engineered iPSCs comprising one or more genetic modifications as provided herein are used to derive hematopoietic cell lineages or any other specific cell types in vitro, wherein the derived non-pluripotent cells retain the functional genetic modifications including targeted editing at the selected site(s). In some embodiments, the genome-engineered iPSCs used to derive hematopoietic cell lineages or any other specific cell types in vitro are master cell bank cells that are cryopreserved and thawed right before their usage. In one embodiment, the genome-engineered iPSC-derived cells include, but are not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages, wherein the cells derived from the genome-engineered iPSCs retain the functional genetic modifications including targeted editing at the desired site(s).

Applicable differentiation methods and compositions for obtaining iPSC-derived hematopoietic cell lineages include those depicted in, for example, International Pub. No. WO2017/078807, the disclosure of which is incorporated herein by reference. As provided, the methods and compositions for generating hematopoietic cell lineages are through definitive hemogenic endothelium (HE) derived from pluripotent stem cells, including iPSCs under serum-free, feeder-free, and/or stromal-free conditions and in a scalable and monolayer culturing platform without the need of EB formation. Cells that may be differentiated according to the provided methods range from pluripotent stem cells, to progenitor cells that are committed to particular terminally differentiated cells and transdifferentiated cells, and to cells of various lineages directly transitioned to hematopoietic fate without going through a pluripotent intermediate. Similarly, the cells that are produced by differentiating stem cells range from multipotent stem or progenitor cells, to terminally differentiated cells, and to all intervening hematopoietic cell lineages.

The methods for differentiating and expanding cells of the hematopoietic lineage from pluripotent stem cells in monolayer culturing comprise contacting the pluripotent stem cells with a BMP pathway activator, and optionally, bFGF. As provided, the pluripotent stem cell-derived mesodermal cells are obtained and expanded without forming embryoid bodies from pluripotent stem cells. The mesodermal cells are then subjected to contact with a BMP pathway activator, bFGF, and a WNT pathway activator to obtain expanded mesodermal cells having definitive hemogenic endothelium (HE) potential without forming embryoid bodies from the pluripotent stem cells. By subsequent contact with bFGF, and optionally, a ROCK inhibitor, and/or a WNT pathway activator, the mesodermal cells having definitive HE potential are differentiated to definitive HE cells, which are also expanded during differentiation.

The methods provided herein for obtaining cells of the hematopoietic lineage are superior to EB-mediated pluripotent stem cell differentiation, because EB formation leads to modest to minimal cell expansion, does not allow monolayer culturing which is important for many applications requiring homogeneous expansion and homogeneous differentiation of the cells in a population, and is laborious and of low efficiency.

The provided monolayer differentiation platform facilitates differentiation towards definitive hemogenic endothelium resulting in the derivation of hematopoietic stem cells and differentiated progeny such as T, B, NKT and NK cells. The monolayer differentiation strategy combines enhanced differentiation efficiency with large-scale expansion, and enables the delivery of a therapeutically relevant number of pluripotent stem cell-derived hematopoietic cells for various therapeutic applications. Further, monolayer culturing using the methods provided herein leads to functional hematopoietic lineage cells that enable a full range of in vitro differentiation, ex vivo modulation, and in vivo long term hematopoietic self-renewal, reconstitution and engraftment. As provided, the iPSC-derived hematopoietic lineage cells include, but are not limited to, definitive hemogenic endothelium, hematopoietic multipotent progenitor cells, hematopoietic stem and progenitor cells, T cell progenitors, NK cell progenitors, T cells, NK cells, NKT cells, B cells, macrophages, and neutrophils.

The method for directing differentiation of pluripotent stem cells into cells of a definitive hematopoietic lineage, comprises: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential.

In some embodiments, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs, or naïve iPSCs, or iPSCs comprising one or more genetic imprints; and the one or more genetic imprints comprised in the iPSCs are retained in the hematopoietic cells differentiated therefrom. In some embodiments of the method for directing differentiation of pluripotent stem cells into cells of a hematopoietic lineage, the differentiation of the pluripotent stem cells into cells of hematopoietic lineage is void of generation of embryoid bodies and is in a monolayer culturing form.

In some embodiments of the above method, the obtained pluripotent stem cell-derived definitive hemogenic endothelium cells are CD34⁺. In some embodiments, the obtained definitive hemogenic endothelium cells are CD34⁺CD43⁻. In some embodiments, the definitive hemogenic endothelium cells are CD34⁺CD43⁻CXCR4⁻CD73⁻. In some embodiments, the definitive hemogenic endothelium cells are CD34⁺CXCR4⁻CD73. In some embodiments, the definitive hemogenic endothelium cells are CD34⁺CD43⁻CD93. In some embodiments, the definitive hemogenic endothelium cells are CD34⁺CD93⁻.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34⁺CD45⁺CD7⁺. In some embodiments of the method, the pluripotent stem cell-derived T cell progenitors are CD45⁺CD7⁺.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3⁻CD45⁺CD56⁺CD7⁺. In some embodiments, the pluripotent stem cell-derived NK cells are CD3⁻CD45⁺CD56⁺, and optionally further defined by being NKp46⁺, CD57⁺ and CD16⁺.

In some embodiments, the genome-engineered iPSC-derived cells obtained from the above methods comprise one or more inducible suicide gene integrated at one or more desired integration sites comprising AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR a or R constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, and TIGIT, or other loci meeting the criteria of a genome safe harbor. In some other embodiments, the genome-engineered iPSC-derived cells comprise polynucleotides encoding safety switch proteins, targeting modality, receptors, signaling molecules, transcription factors, pharmaceutically active proteins and peptides, drug target candidates, or proteins promoting trafficking, homing, viability, self-renewal, persistence, and/or survival of stem cells and/or progenitor cells. In some embodiments, the genome-engineered iPSC-derived cells comprising one or more suicide genes further comprise one or more in/dels comprised in one or more endogenous genes associated with immune response regulation and mediation, including, but not limited to, checkpoint genes, endogenous T cell receptor genes, and MHC class I suppressor genes. In one embodiment, the genome-engineered iPSC-derived cells comprising one or more suicide genes further comprise an in/del in B2M gene, wherein the B2M is knocked-out.

Additionally, applicable dedifferentiation methods and compositions for obtaining genomic-engineered hematopoietic cells of a first fate to genomic-engineered hematopoietic cells of a second fate include those depicted in, for example, International Pub. No. WO2011/159726, the disclosure of which is incorporated herein by reference. The method and composition provided therein allows partially reprogramming a starting non-pluripotent cell to a non-pluripotent intermediate cell by limiting the expression of endogenous Nanog gene during reprogramming; and subjecting the non-pluripotent intermediate cell to conditions for differentiating the intermediate cell into a desired cell type.

V. Therapeutic Use of Derivative Immune Cells with Exogenous Functional Modalities Differentiated from Genetically Engineered iPSCs

The present invention provides, in some embodiments, a composition comprising an isolated population or subpopulation of functionally enhanced derivative immune cells that have been differentiated from genomically engineered iPSCs using the methods and compositions as disclosed. In some embodiments, the iPSCs of the composition comprise one or more targeted genetic edits as disclosed, which are retainable in the iPSC-derived effector cells, wherein the genetically engineered iPSCs and derivative cells thereof are suitable for cell-based adoptive therapies. In one embodiment, the isolated population or subpopulation of genetically engineered effector cells of the composition comprises iPSC-derived CD34⁺ cells. In one embodiment, the isolated population or subpopulation of genetically engineered effector cells of the composition comprises iPSC-derived HSC cells. In one embodiment, the isolated population or subpopulation of genetically engineered effector cells of the composition comprises iPSC-derived proT or T cells. In one embodiment, the isolated population or subpopulation of genetically engineered effector cells of the composition comprises iPSC-derived proNK or NK cells. In one embodiment, the isolated population or subpopulation of genetically engineered effector cells of the composition comprises iPSC-derived immune regulatory cells or myeloid derived suppressor cells (MDSCs).

In some embodiments of the composition, the iPSC-derived genetically engineered effector cells are further modulated ex vivo for improved therapeutic potential. In one embodiment of the composition, an isolated population or subpopulation of genetically engineered effector cells that have been derived from iPSCs comprises an increased number or ratio of naïve T cells, stem cell memory T cells, and/or central memory T cells. In one embodiment of the composition, the isolated population or subpopulation of genetically engineered effector cells that have been derived from iPSCs comprises an increased number or ratio of type I NKT cells. In another embodiment of the composition, the isolated population or subpopulation of genetically engineered effector cells that have been derived from iPSCs comprises an increased number or ratio of adaptive NK cells. In some embodiments of the composition, the isolated population or subpopulation of genetically engineered CD34⁺ cells, HSC cells, T cells, NK cells, or myeloid derived suppressor cells derived from iPSCs are allogeneic. In some other embodiments of the composition, the isolated population or subpopulation of genetically engineered CD34⁺ cells, HSC cells, T cells, NK cells, or MDSCs derived from iPSC are autologous.

In some embodiments of the composition, the iPSC for differentiation comprises genetic imprints selected to convey desirable therapeutic attributes in derived effector cells, provided that cell development biology during differentiation is not disrupted, and provided that the genetic imprints are retained and functional in the differentiated hematopoietic cells derived from said iPSC.

In some embodiments of the composition, the genetic imprints of the pluripotent stem cells comprise (i) one or more genetically modified modalities obtained through genomic insertion, deletion or substitution in the genome of the pluripotent cells during or after reprogramming a non-pluripotent cell to iPSC; or (ii) one or more retainable therapeutic attributes of a source specific immune cell that is donor-, disease-, or treatment response-specific, and wherein the pluripotent cells are reprogrammed from the source specific immune cell, wherein the iPSC retain the source therapeutic attributes, which are also comprised in the iPSC-derived hematopoietic lineage cells.

In some embodiments of the composition, the genetically modified modalities comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some embodiments of the composition, the genetically modified iPSC and the derivative cells thereof comprise a genotype listed in Table 1. In some other embodiments of the composition, the genetically modified iPSC and the derivative cells thereof comprising a genotype listed in Table 1 further comprise additional genetically modified modalities comprising (1) disruption of one or more of B2M, TAP1, TAP2, tapasin, NLRC5, CIITA, RFXANK, RFX5, RFXAP, TCRα or TCRβ constant region (TRAC or TRBC), NKG2A, NKG2D, CD38, CD25, CD69, CD71, CD44, CD54, CD56, CD58, OX40, 4-1BB, CIS, CBL-B, SOCS2, PD1, CTLA4, LAG3, TIM3 or TIGIT; and/or (2) introduction of one or more of HLA-E, HLA-G, 4-1BBL, CD3, CD4, CD8, CD47, CD113, CD131, CD137, CD80, PDL1, A_{2A}R, CAR, TCR, Fc receptor, or surface triggering receptors for coupling with bi- or multi-specific or universal engagers.

In still some other embodiments of the composition, the iPSC-derived hematopoietic lineage cells comprise the therapeutic attributes of the source specific immune cell relating to one or more of: (i) increased cytotoxicity; (ii) improved persistency and/or survival; (iii) enhanced ability in migrating, and/or activating or recruiting bystander immune cells, to tumor sites; (iv) improved tumor penetration; (v) enhanced ability to reduce tumor immunosuppression; (vi) improved ability in rescuing tumor antigen escape; (vii) controlled apoptosis; (viii) enhanced or acquired ADCC; and (ix) ability to avoid fratricide, in comparison to its counterpart primary cell obtained from peripheral blood, umbilical cord blood, or any other donor tissues without the same genetic edit(s). In some embodiments of the composition, the iPSC-derived hematopoietic lineage cells additionally comprise the therapeutic attributes of promoting homing or trafficking and retension of the effector cells at a tumor site.

In some embodiments of the composition, the iPSC-derived hematopoietic cells comprising a genotype listed in Table 1 express at least one cytokine and/or its receptor comprising IL2, IL4, IL6, IL7, IL9, IL10, IL11, IL12, IL15, IL18, or IL21, or any modified protein thereof, and express at least a CAR. In some embodiments of the composition, the cells express at least one cytokine and/or its receptor comprising IL2, IL4, IL7, IL9, IL15, and IL21. In some embodiments of the composition, the cells express at least one cytokine and/or its receptor comprising IL7 or IL15. In some embodiments of the composition, the engineered expression of the cytokine(s) and the CAR(s) is NK cell specific. In some other embodiments of the composition, the engineered expression of the cytokine(s) and the CAR(s) is T cell specific. In some embodiments of the composition, the iPSC-derived hematopoietic effector cells are antigen specific. In some embodiments of the composition, the antigen specific derivative effector cells target a liquid tumor. In some embodiments of the composition, the antigen specific derivative effector cells target a solid tumor. In some embodiments of the composition, the antigen specific iPSC-derived hematopoietic effector cells are capable of rescuing tumor antigen escape. Additionally, the present application makes possible a combined therapeutic approach by providing rationally designed effector cells capable of synergize with a tumor sensitizing procedure that upregulates tumor cell expression of a matching chemokine to augment effector cell tumor site homing, trafficking and retention, which contributes to increased effector cell cytotoxicity and persistency.

As provided in this application, exposing a tumor cell to a sensitizing agent (e.g., radiation) elevates secretion and/or surface expression of stress ligands including, but limited to, the chemokine IL8, by the tumor cells. Tumor preconditioning by sensitization, as described herein therefore provides an additional strategy to further enhance the therapeutic efficacy of the effector cells overexpressing CXCR2 or a variant thereof. Without being limited by theory, tumor sensitization may be utilized to overcome tumor resistance by modulating potential tumorigenic mechanisms (including, but not limited to cell cycle progression, inflammation, proliferation, apoptosis, invasion, perfusion, metastasis, and angiogenesis) to make the tumor cells more susceptible to activities of another selective drugs, such as the allogeneic effector cells with desired engineered therapeutic attributes as described herein, thereby enhancing the efficacy of the therapeutic effector cells targeting the tumor.

Without being bound by theory, exemplary sensitizing agents useful in compositions and methods disclosed herein include, but are not limited to, radiation therapy, radiopharmaceuticals, or chemotherapeutic agents. Thus, the above-discussed compositions of the invention may further comprise a sensitizing agent, as described above. In various embodiments, the sensitizing agent increases secretion and/or surface expression of a chemokine including CXCL8 by a tumor cell upon contact therewith.

Embodiments of radiation therapy include, but are not limited to, external beam radiation therapy, wherein high-energy beams (e.g., x-rays, gamma rays, photons, protons, neutrons, ions, and any other forms of energy applicable to such treatments) are produced by a machine and aimed at the tumor; brachytherapy, wherein seeds, ribbons, or capsules that contain or are otherwise linked to a radiation source/particle are placed in or near a tumor or cancer cell. Embodiments of radioactive drugs (e.g., radiopharmaceuticals or radionuclides, including radiopeptides) comprise a radioactive compound linked to a targeting molecule (e.g., an antibody conjugate).

In various embodiments, the amount of radiation agent being exposed to, or contacted with, a cancer or tumor cell ranges from about 0.0001 Gy to about 80Gy. Thus, in some embodiments, the amount of sensitizing agent provided to a subject and/or included in the compositions provided herein is at least about 0.0001 Gy, at least about 0.0005 Gy, at least about 0.001 Gy, at least about 0.0015 Gy, at least about 0.01 Gy, at least about 0.015 Gy, at least about 0.1 Gy, at least about 0.15 Gy, at least about 1.0 Gy, at least about 1.5 Gy, at least about 10.0 Gy, at least about 15 Gy, at least about 20.0 Gy, at least about 25.0 Gy, at least about 30.0 Gy, at least about 35.0 Gy, at least about 40.0 Gy, at least about 45.0 Gy, at least about 50.0 Gy, at least about 55.0 Gy, at least about 60.0 Gy, at least about 65.0 Gy, at least about 70.0 Gy, at least about 75.0 Gy, at least about 80.0 Gy or any range in-between. In particular embodiments, the amount of sensitizing agent is about 25.0 Gy.

Examples of radioactive compounds useful as radiopharmaceuticals include, but are not limited to calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iorn-59, krypton-81m, lutetium-177, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90.

Exemplary chemotherapeutic agents that can be potentially used for tumor cell sensitization include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable chemotherapeutic agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y, 1995) or through the National Cancer Institute website (fda.gov/cder/cancer/druglistfrarne.htm), both as updated from time to time.

A variety of diseases may be ameliorated by introducing the derivative effector cells and/or the compositions of the invention to a subject suitable for adoptive cell therapy. In some embodiments, the iPSC-derived hematopoietic cells or the compositions as provided herein are for allogeneic adoptive cell therapies. Additionally, the present invention provides, in some embodiments, therapeutic use of the above immune cells and/or therapeutic compositions and/or combination therapies by introducing the cells or composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus. Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes. Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus. Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's). Examples of viral infections include, but are not limited to, HIV- (human immunodeficiency virus), HSV- (herpes simplex virus), KSHV- (Kaposi's sarcoma-associated herpesvirus), RSV- (Respiratory Syncytial Virus), EBV- (Epstein-Barr virus), CMV- (cytomegalovirus), VZV (Varicella zoster virus), adenovirus-, a lentivirus-, a BK polyomavirus-associated disorders.

The treatment using the derived hematopoietic lineage cells of embodiments disclosed herein, or the compositions provided herein, could be carried out upon symptom presentation, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; and inhibiting the disease, i.e., arresting its development; or relieving the disease, i.e., causing regression of the disease. The therapeutic agent(s) and/or compositions may be administered before, during or after the onset of a disease or an injury. Treatment of ongoing disease, where the treatment stabilizes or reduces the undesirable clinical symptoms of the patient, is also of particular interest. In particular embodiments, the subject in need of a treatment has a disease, a condition, and/or an injury that can be contained, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

When evaluating responsiveness to the treatment comprising the derived hematopoietic lineage cells of embodiments disclosed herein, the response can be measured by criteria comprising at least one of: clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST (Response Evaluation Criteria In Solid Tumors) criteria.

The therapeutic composition comprising iPSC-derived hematopoietic lineage cells as disclosed herein can be administered to a subject before, during, and/or after other treatments, including sensitization of cancer or tumor cells, as described above. As such a method of combinational therapy can involve the administration or preparation of iPSC-derived effector cells before, during, and/or after the use of one or more additional therapeutic agents. As provided above, the one or more additional therapeutic agents comprise a peptide, a cytokine, a checkpoint inhibitor, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). The administration of the iPSC-derived immune cells can be separated in time from the administration of an additional therapeutic agent by hours, days, or even weeks. Additionally, or alternatively, the administration can be combined with other biologically active agents or modalities such as, but not limited to, an antineoplastic agent, a non-drug therapy, such as, surgery.

In some embodiments of a combinational cell therapy, the therapeutic combination comprises the iPSC-derived effector cells provided herein and an additional therapeutic agent that is an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived hematopoietic lineage cells to enhance their killing ability. In some embodiments, the antibodies suitable for combinational treatment as an additional therapeutic agent to the administered iPSC-derived hematopoietic lineage cells include, but are not limited to, anti-CD20 antibodies (e.g., rituximab, veltuzumab, ofatumumab, ublituximab, ocaratuzumab, obinutuzumab), anti-HER2 antibodies (e.g., trastuzumab, pertuzumab), anti-CD52 antibodies (e.g., alemtuzumab), anti-EGFR antibodies (e.g., cetuximab), anti-GD2 antibodies (e.g., dinutuximab), anti-PDL1 antibodies (e.g., avelumab), anti-CD38 antibodies (e.g., daratumumab, isatuximab, MOR202), anti-CD123 antibodies (e.g., 7G3, CSL362), anti-SLAMF7 antibodies (elotuzumab), MICA/B antibodies (7C6, 6F11, 1C2), and their humanized or Fc modified variants or fragments or their functional equivalents or biosimilars. The present invention provides therapeutic compositions comprising effector cells, including the iPSC-derived hematopoietic lineage cells, having a genotype listed in Table 1 and an additional therapeutic agent that is an antibody, or an antibody fragment, as described above.

In some embodiments, the additional therapeutic agent comprises one or more checkpoint inhibitors. Checkpoints are referred to cell molecules, often cell surface molecules, capable of suppressing or downregulating immune responses when not inhibited. Checkpoint inhibitors are antagonists capable of reducing checkpoint gene expression or gene products, or deceasing activity of checkpoint molecules. Suitable checkpoint inhibitors for combination therapy with the derivative effector cells include, but are not limited to, antagonists of PD1 (Pdcdl, CD279), PDL-1 (CD274), TIM3 (Havcr2), TIGIT (WUCAM and Vstm3), LAG3 (CD223), CTLA4 (CD152), 2B4 (CD244), 4-1BB (CD137), 4-1BBL (CD137L), A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

Some embodiments of the combination therapy comprising the provided derivative effector cells further comprise at least one inhibitor targeting a checkpoint molecule. Some other embodiments of the combination therapy with the provided derivative effector cells comprise two, three or more inhibitors such that two, three, or more checkpoint molecules are targeted. In some embodiments, the effector cells for combination therapy as described herein are derivative NK cells as provided. In some embodiments, the effector cells for combination therapy as described herein are derivative T cells. In some embodiments, the derivative NK or T cells for combination therapies are functionally enhanced as provided herein. In some embodiments, the two, three or more checkpoint inhibitors may be administered in a combination therapy with, before, or after the administering of the derivative effector cells. In some embodiments, the two or more checkpoint inhibitors are administered at the same time, or one at a time (sequential). The present invention provides therapeutic compositions comprising effector cells, including the iPSC-derived effector cells, having a genotype listed in Table 1 and one or more checkpoint inhibitors, as described above.

In some embodiments, the antagonist inhibiting any of the above checkpoint molecules is an antibody. In some embodiments, the checkpoint inhibitory antibodies may be murine antibodies, human antibodies, humanized antibodies, a camel Ig, a single variable new antigen receptor (VNAR), a shark heavy-chain-only antibody (Ig NAR), chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab, avelumab, durvalumab, ipilimumab, IPH4102, IPH43, IPH33, lirimumab, monalizunab, nivolumab, pembrolizumab, and their derivatives or functional equivalents.

The combination therapies comprising the derivative effector cells and one or more check inhibitors are applicable to treatment of liquid and solid cancers, including but not limited to cutaneous T-cell lymphoma, non-Hodgkin lymphoma (NHL), Mycosis fungoides, Pagetoid reticulosis, Sezary syndrome, Granulomatous slack skin, Lymphomatoid papulosis, Pityriasis lichenoides chronica, Pityriasis lichenoides et varioliformis acuta, CD30⁺ cutaneous T-cell lymphoma, Secondary cutaneous CD30⁺ large cell lymphoma, non-mycosis fungoides CD30 cutaneous large T-cell lymphoma, Pleomorphic T-cell lymphoma, Lennert lymphoma, subcutaneous T-cell lymphoma, angiocentric lymphoma, blastic NK-cell lymphoma, B-cell Lymphomas, hodgkins lymphoma (HL), Head and neck tumor; Squamous cell carcinoma, rhabdomyocarcoma, Lewis lung carcinoma (LLC), non-small cell lung cancer, esophageal squamous cell carcinoma, esophageal adenocarcinoma, renal cell carcinoma (RCC), colorectal cancer (CRC), acute myeloid leukemia (AML), breast cancer, gastric cancer, prostatic small cell neuroendocrine carcinoma (SCNC), liver cancer, glioblastoma, liver cancer, oral squamous cell carcinoma, pancreatic cancer, thyroid papillary cancer, intrahepatic cholangiocellular carcinoma, hepatocellular carcinoma, bone cancer, metastasis, and nasopharyngeal carcinoma.

In some embodiments, other than the derivative effector cells as provided herein, a combination for therapeutic use comprises one or more additional therapeutic agents comprising a chemotherapeutic agent or a radioactive moiety. “Chemotherapeutic agent” refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, examples of which are known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y, 1995) or through the National Cancer Institute website (fda.gov/cder/cancer/druglistfrarne.htm), both as updated from time to time.

Immunomodulatory drugs (IMiDs) such as thalidomide, lenalidomide, and pomalidomide stimulate both NK cells and T cells. As provided herein, IMiDs may be used with the iPSC-derived therapeutic immune cells for cancer treatments.

Other than an isolated population of iPSC-derived hematopoietic lineage cells included in the therapeutic compositions, the compositions suitable for administration to a subject/patient can further include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of embodiments of the present invention (see, e.g., Remington's Pharmaceutical Sciences, 17^{th} ed. 1985, the disclosure of which is hereby incorporated by reference in its entirety).

In one embodiment, the therapeutic composition comprises the iPSC-derived T cells made by the methods and composition disclosed herein. In one embodiment, the therapeutic composition comprises the pluripotent cell derived NK cells made by the methods and composition disclosed herein. In one embodiment, the therapeutic composition comprises the iPSC-derived CD34⁺ HE cells made by the methods and composition disclosed herein. In one embodiment, the therapeutic composition comprises the pluripotent cell derived HSCs made by the methods and composition disclosed herein. In one embodiment, the therapeutic composition comprises the pluripotent cell derived MDSC made by the methods and composition disclosed herein. A therapeutic composition comprising a population of iPSC-derived hematopoietic lineage cells as disclosed herein can be administered separately by intravenous, intraperitoneal, enteral, or tracheal administration methods or in combination with other suitable compounds to affect the desired treatment goals.

These pharmaceutically acceptable carriers and/or diluents can be present in amounts sufficient to maintain a pH of the therapeutic composition of between about 3 and about 10. As such, a buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures of embodiments of the present invention. Such compositions are suitable for administration to human subjects. Generally speaking, any medium that supports the maintenance, growth, and/or health of the iPSC-derived effector cells in accordance with embodiments of the invention are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of media suitable for use in the present invention and that there are many suitable media known and available to those in the art.

The iPSC-derived hematopoietic lineage cells can have at least 50%, 60%, 70%, 80%, 90%, 95%, 98%, or 99% T cells, NK cells, NKT cells, proT cells, proNK cells, CD34⁺ HE cells, HSCs, B cells, myeloid-derived suppressor cells (MDSCs), regulatory macrophages, regulatory dendritic cells, or mesenchymal stromal cells. For example, in some embodiments, the iPSC-derived hematopoietic lineage cells can have at least 50%, 60%, 70%, 80%, 90%, 95%, 98%, or 99% NK cells. For example, in some embodiments, the iPSC-derived hematopoietic lineage cells can have at least 50%, 60%, 70%, 80%, 90%, 95%, 98%, or 99% T cells. In some embodiments, the iPSC-derived hematopoietic lineage cells have about 95% to about 100% T cells, NK cells, proT cells, proNK cells, CD34⁺ HE cells, or myeloid-derived suppressor cells (MDSCs). In some embodiments, the iPSC-derived hematopoietic lineage cells have about 95% to about 100% NK cells. In some embodiments, the iPSC-derived hematopoietic lineage cells have about 95% to about 100% T cells. In some embodiments, the present invention provides therapeutic compositions having purified T cells or NK cells, such as a composition having an isolated population of about 95% T cells, NK cells, proT cells, proNK cells, CD34⁺ HE cells, or myeloid-derived suppressor cells (MDSCs) to treat a subject in need of the cell therapy. In some embodiments, the present invention provides therapeutic compositions having purified NK cells, such as a composition having an isolated population of about 95% NK cells to treat a subject in need of the cell therapy. In some embodiments, the present invention provides therapeutic compositions having purified T cells, such as a composition having an isolated population of about 95% T cells to treat a subject in need of the cell therapy.

One aspect of the present application provides a method of treating a subject in need by administering one or more therapeutic doses of effector cells comprising at least a CXCR2 or a variant thereof, and optionally one or more of a CAR, exogenous CD16 or a variant thereof, CD38 knockout, TCR^{neg} and an exogenous cytokine signaling complex. In some embodiments, the present application provides a method of treating a subject having cancer or a tumor by first sensitizing the cancer or tumor cell in the subject to increase or enhance secretion and/or surface expression of one or more chemokines that are ligands to CXCR2, as compared to chemokine secretion and/or surface expression prior to such contacting/exposure. Following sensitization of the cancer or tumor cell an effector cell or population thereof, as described above, is given/administered to the subject, wherein the effector cell comprises at least a CXCR2 or a variant thereof, and optionally one or more of a CAR, exogenous CD16 or a variant thereof, CD38 knockout, TCR^{neg} and an exogenous cytokine signaling complex. In various embodiments, the effector cell or population thereof may be provided prior to or concurrently with one or more additional therapeutic agents, as described above.

Another aspect of the present application provides a method of treating a subject in need using a combinational cell therapy. In some embodiments of the combinational cell therapy, the method of treating a subject in need comprises administering one or more therapeutic doses of effector cells comprising at least a CXCR2 or a variant thereof, and optionally one or more of a CAR, exogenous CD16 or a variant thereof, CD38 knockout, TCR^{neg} and an exogenous cytokine signaling complex, and one or more therapeutic agents comprising a peptide, a cytokine, a checkpoint inhibitor, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD), and optionally preconditioning tumor cells in the subject by administering a sensitizing agent. In some embodiments, the combinational cell therapy, or composition used therefor, comprises a population of effector cells derived from genomically engineered iPSCs and one or more therapeutic agents, wherein the engineered iPSCs and the derived effector cells comprise CXCR2 or a variant thereof, and optionally a CAR, exogenous CD16 or a variant thereof, CD38 knockout, or other edits described herein, or a genotype listed in Table 1. In some embodiments of the method of combinational cell therapy, the method comprises preconditioning tumor cells in the subject by administering a sensitizing agent, wherein the sensitizing agent comprises radiation therapy, radiopharmaceuticals, or chemotherapeutic agents as provided herein. In various embodiments, preconditioning of the tumor cells in the subject occurs prior to, or concurrently with administering the one or more therapeutic doses of effector cells described herein.

As a person of ordinary skill in the art would understand, both autologous and allogeneic hematopoietic lineage cells derived from iPSC based on the methods and compositions provided herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject, wherein the derived hematopoietic lineage cells are NK cells or T cell with HLA-I and/or HLA-II deficiency.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to 1.67×10⁶ cells/kg for a 60 kg patient/subject.

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between. In some embodiments, the multi-dose treatment comprises three, four, or five, once-weekly doses. In some embodiments of the multi-dose treatment comprising three, four, or five, once-weekly doses further comprise an observation period for determining whether additional single or multi doses are needed.

The compositions comprising a population of derived hematopoietic lineage cells of the invention can be sterile, and can be suitable and ready for administration (i.e., can be administered without any further processing) to human patients/subjects. A cell-based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more agents including small chemical molecules. The compositions and methods for modulating immune cells including iPSC-derived effector cells are described in greater detail, for example, in International Pub. No. WO2017/127755, the relevant disclosure of which is incorporated herein by reference. For derived hematopoietic lineage cells that are genetically engineered to express recombinant TCR or CAR, the cells can be activated and expanded using methods as described, for example, in U.S. Pat. No. 6,352,694.

In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Pub. Nos. 2004/0101519 and 2006/0034810, the disclosures of which are incorporated by reference, for artificial antigen presenting cells (aAPCs) that are contemplated for use in activating and expanding T lymphocytes in embodiments of the present invention.

Some variation in dosage, frequency, and protocol will necessarily occur depending on the condition of the subject being treated. The person responsible for administration will, in any event, determine the appropriate dose, frequency and protocol for the individual subject.

### EXAMPLES

The following examples are offered by way of illustration and not by way of limitation.

### Example 1—Materials and Methods

To effectively select and test suicide systems under the control of various promoters in combination with different safe harbor loci integration strategies, a proprietary hiPSC platform of the applicant was used, which enables single cell passaging and high-throughput, 96-well plate-based flow cytometry sorting, to allow for the derivation of clonal hiPSCs with single or multiple genetic modulations.

hiPSCMaintenance in Small Molecule Culture: hiPSCs were routinely passaged as single cells once confluency of the culture reached 75%-90%. For single-cell dissociation, hiPSCs were washed with PBS (Mediatech) and treated with Accutase (Millipore) for 3-5 min at 37° C. The single-cell suspension was then mixed with conventional medium, centrifuged, resuspended in FMM, and plated on Matrigel-coated surface. Passages were typically 1:6-1:8, transferred tissue culture plates previously coated with Matrigel and fed every 2-3 days with FMM. Cell cultures were maintained in a humidified incubator set at 37° C. and 5-10% C02.

Human iPSC engineering with ZFN, CRISPR for targeted editing of modalities of interest: Using ROSA26 targeted insertion as an example, for ZFN mediated genome editing, 2 million iPSCs were transfected with a mixture of 2.5 μg ZFN-L, 2.5 μg ZFN-R and 5 μg donor construct, for AAVS1 targeted insertion. For CRISPR mediated genome editing, 2 million iPSCs were transfected with a mixture of 5 μg ROSA26-gRNA/Cas9 and 5 μg donor construct, for ROSA26 targeted insertion. Transfection was done using Neon transfection system (Life Technologies). On day 2 or 3 after transfection, transfection efficiency was measured using flow cytometry if the plasmids contain artificial promoter-driven GFP and/or RFP expression cassette.

Bulk sort and clonal sort of genome-edited iPSCs: iPSCs with genomic targeted editing using ZFN or CRISPR-Cas9 were bulk sorted and clonal sorted of GFP⁺SSEA4⁺TRA181⁺ iPSCs. Single cell dissociated targeted iPSC pools were resuspended in staining buffer containing Hanks' Balanced Salt Solution (MediaTech), 4% fetal bovine serum (Invitrogen), lx penicillin/streptomycin (Mediatech) and 10 mM Hepes (Mediatech); made fresh for optimal performance. Conjugated primary antibodies, including SSEA4-PE, TRA181-Alexa Fluor-647 (BD Biosciences), were added to the cell solution. The solution was washed in staining buffer, spun and resuspended in staining buffer containing 10 μM Thiazovivn for flow cytometry sorting. Flow cytometry sorting was performed on FACS Aria II (BD Biosciences). Upon completion of the sort, the 96-well plates were incubated. Colony formation was detected as early as day 2 and most colonies were expanded between days 7-10 post sort. In the first passage, wells were washed with PBS and dissociated with 30 μL Accutase. The dissociated colony was transferred to another well of a 96-well plate previously coated with 5× Matrigel. Subsequent passages were done routinely. Each clonal cell line was analyzed for GFP fluorescence level and TRA1-81 expression level. Clonal lines with near 100% GFP⁺ and TRA1-81⁺ were selected for further screening and analysis including but not limited to off-target editing, and/or karyotype of the engineered iPSCs, before the clonal population is cryopreserved and serves as a master cell bank. Flow cytometry analysis was performed on Guava EasyCyte 8 HT (Millipore) and analyzed using Flowjom (FlowJo, LLC).

### Example 2—Immune Cells Expressing CXCR2 and CAR Demonstrate Enhanced Migration

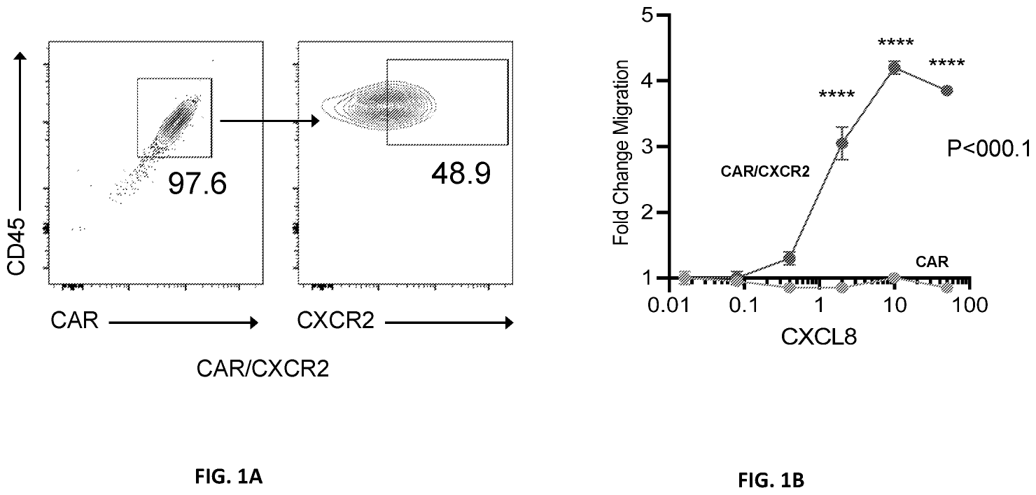

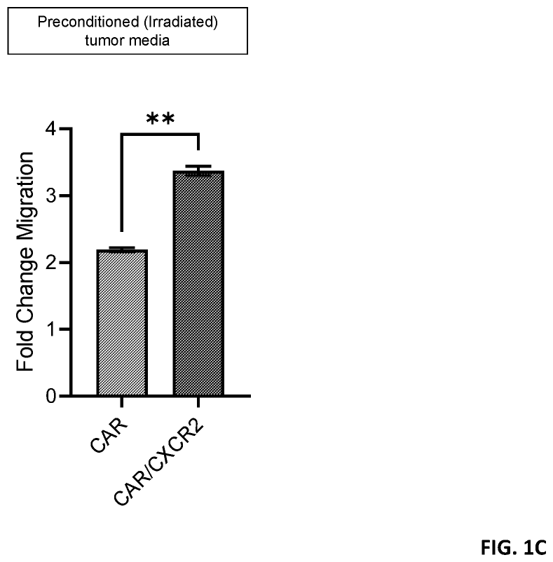

To explore whether overexpressing one particular chemokine receptor in an effector cell could help cell migration and infiltration to solid tumors, primary T cells were engineered using lentiviral constructs to produce CAR-T expressing CXCR2 (CXCR2⁺ CAR-T or CAR/CXCR2 T cell) and CAR-T cells without exogenous CXCR2 (CXCR2⁻ CAR-T or CAR-T). Both CAR-T (not shown) and CAR/CXCR2 T cells expressed >97% CAR, and the CAR/CXCR2 T cells have heterogenous surface expression of CXCR2, as detected by flow cytometry (FIG. 1A).

A transwell migration assay was used to study the functional migration of CXCR2⁺ and CXCR2⁻ CAR-T cells to the CXCR2 ligand, CXCL8 (IL8). As demonstrated in FIG. 1B using fold change of cell migration in responding to CXCL8 at different concentrations, CXCR2⁺ CAR-T cells had significantly higher chemotaxis (P<000.1) to the CXCR2 ligand in a dose dependent manner, as compared to CXCR2⁻ CAR-T cells. A maximal migration of approximately 2-fold over baseline was observed with 10 ng/ml of rhIL8. In addition, as shown in FIG. 1C, CXCR2⁺ CAR-T cells also demonstrated significantly increased chemotaxis in response to irradiated/preconditioned tumor media (supernatant from pre-conditioned tumor lines), with a functional enhancement in migration up to 4-fold compared to non-CXCR2 expressing CAR-T controls (P<0.01). As such, the expression of CXCR2 in CAR-T cells enables specific migration towards CXCL8 and enhanced migration to preconditioned tumor media.

### Example 3—CXCR2⁺ CAR-iT Cells Demonstrate Enhanced Migratory Capabilities without Affecting CXCR3 Sensitivity or Cytolytic Efficacy

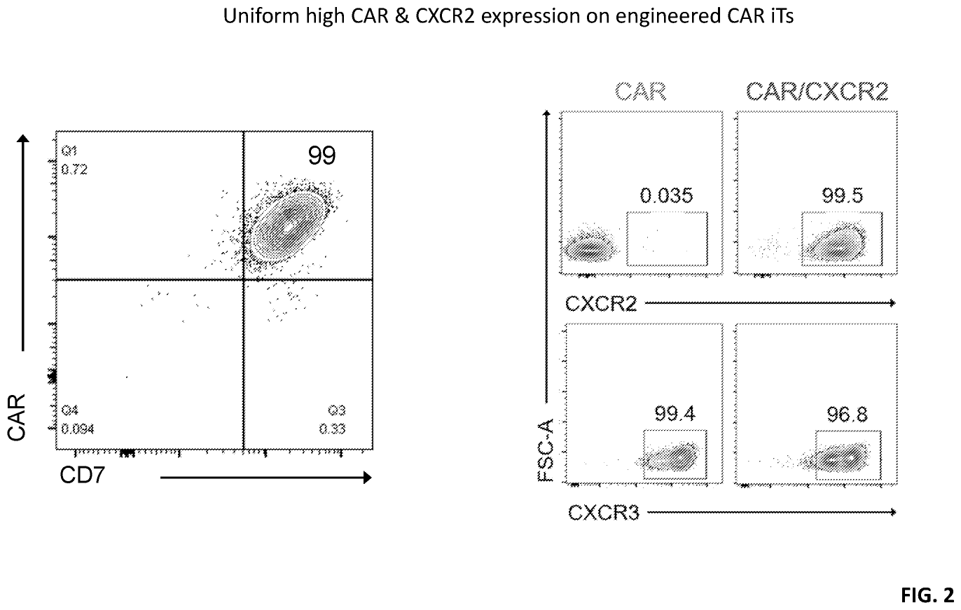

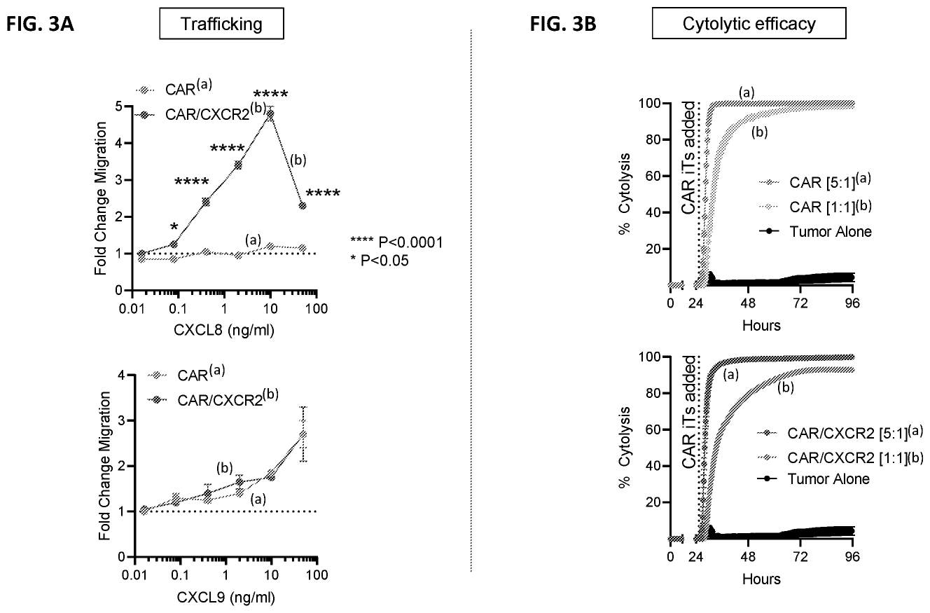

In this set of experiments, induced pluripotent stem cells (iPSCs) were engineered to express CAR and CXCR2 and were subsequently differentiated to T cells to generate iPSC-derived CXCR2⁺ CAR-T cells (CXCR2⁺ CAR-iT cells). In a further embodiment, the CXCR2⁺ CAR-iT cells are also TCR^{neg}, which could be achieved, among other approaches, by inserting the CAR at a constant region of TCR or at a selected gene locus as described herein, to knock out the endogenous TCR expression when generating the engineered iPSC for T cell differentiation. As demonstrated by flow cytometry in FIG. 2, unlike their primary CXCR2⁺ CAR-T cell counterparts that are heterogenous in CXCR2⁺ expression level, the CXCR2⁺ engineered iPSC-derived CAR-T cells were found to express uniformly high levels of CXCR2. Additionally, the CAR-iT cells comprising transduced CXCR2 constitutively express uniformly high levels of CXCR3 (FIG. 2). Like their primary CAR-T cell counterparts, functional chemotaxis of CXCR2⁺ CAR-iT cells was observed in response to CXCL8 (FIG. 3A, top panel) and preconditioned tumor media using a transwell migration assay, and yet remained similarly sensitive to the CXCR3 ligand CXCL9 (FIG. 3A, bottom panel). An evaluation by an xCELLigencem assay is demonstrated in FIG. 3B, where both CAR-iT cells (top panel) and CXCR2⁺ CAR-iT cells (bottom panel) are shown to exert robust cytolytic targeting of tumor targets at the indicated Effector:Target ratios. Therefore, CXCR2 expression did not limit CAR-dependent cytolytic function and the specificity of CAR-iT cells, underscoring the compatibility of this approach.

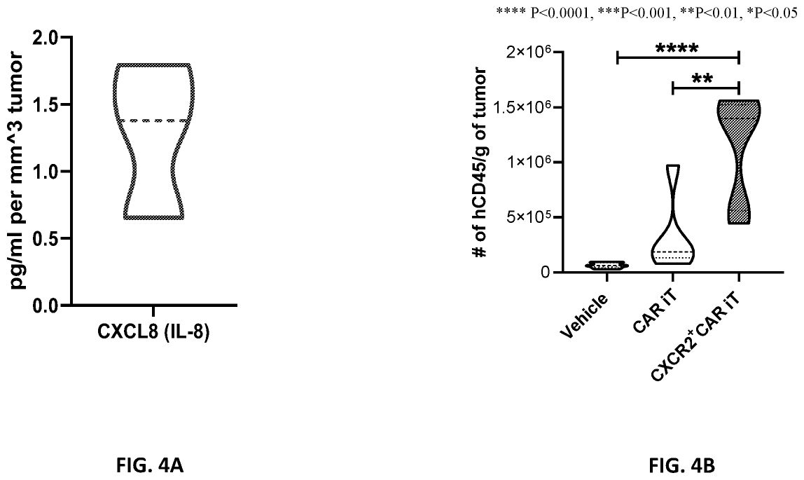

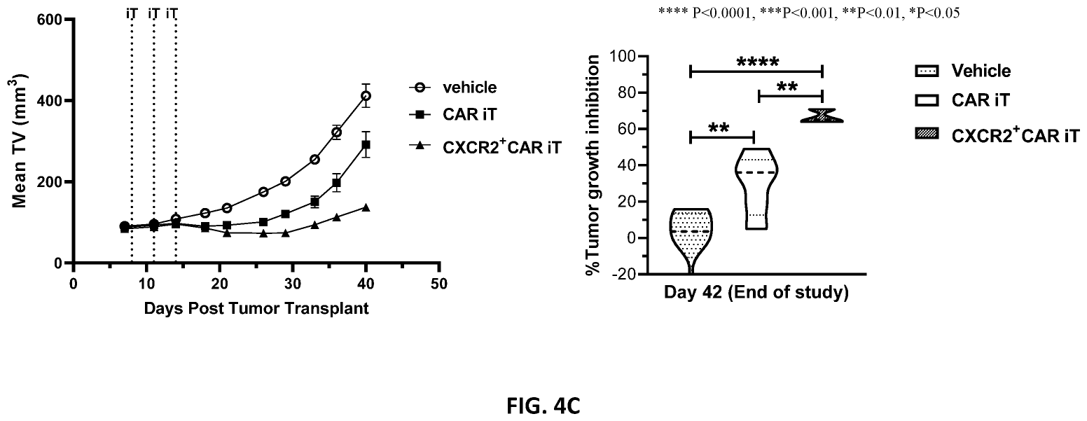

Finally, tumor growth inhibition of the CXCR2⁺ CAR-iT cells was evaluated in vivo in an ovarian xenograft model. 2e6 ovarian tumor cells were injected subcutaneously (s.c.) into NSG mice on Day 0 (N=5 mice per group). FIG. 4A shows CXCL8 levels in serum collected prior to effector dosing. Effector cells (CXCR2⁺ CAR-iTs, CXCR2⁻ CAR-iTs, or vehicle (control)) were administered intravenously (i.v.) at three doses of 10e6 every three days beginning on Day 8, and tumor growth was evaluated during the course of treatment (FIG. 4C). Tumors were harvested on Day 42 and the number of CAR-iTs were determined for each group via flow cytometry analysis (FIG. 4B).

### Example 4—Sensitizing a Tumor Cell Auguments Chemokine Secretion

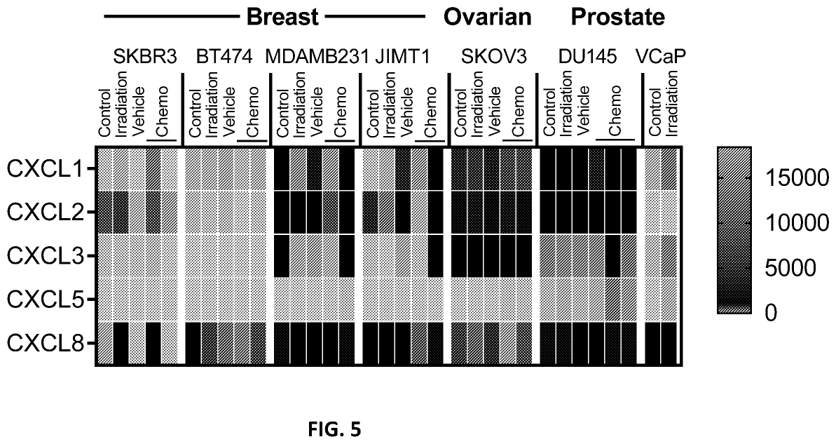

In order to assess whether upregulating IL8 in tumor cells can be leveraged as a mechanism to further enhance CAR-T cell migration and/or infiltration to the tumor site, various cancer cells were used to explore whether sensitization of a tumor cell using chemo- and radio-therapy could increase CXCR2 ligand expression. Selected cancer cell lines (breast, ovarian, and prostate) were either exposed to various chemotherapeutic agents (e.g., Cisplatin, IC₅₀) or were exposed to one cycle of 21 Gy x-ray radiation, and parallel mock and vehicle treated cells were also prepared. After 48 hours, cells and supernatants were collected, processed, and analyzed for RNAseq and ELISA respectively. CXCR2 ligand gene expression profiles, including those of CXCL1, CXCL2, CXCL3, CXCL5 and CXCL8 are shown in FIG. 5 (empirically determined IC₅₀) which demonstrated induced expression in the panel of chemokines across diverse solid tumor indications. Among the tested chemokines, it appeared that CXCL8 gene expression responds to chemotherapy- and radiotherapy-based preconditioning most prominently across the solid cancer indications.

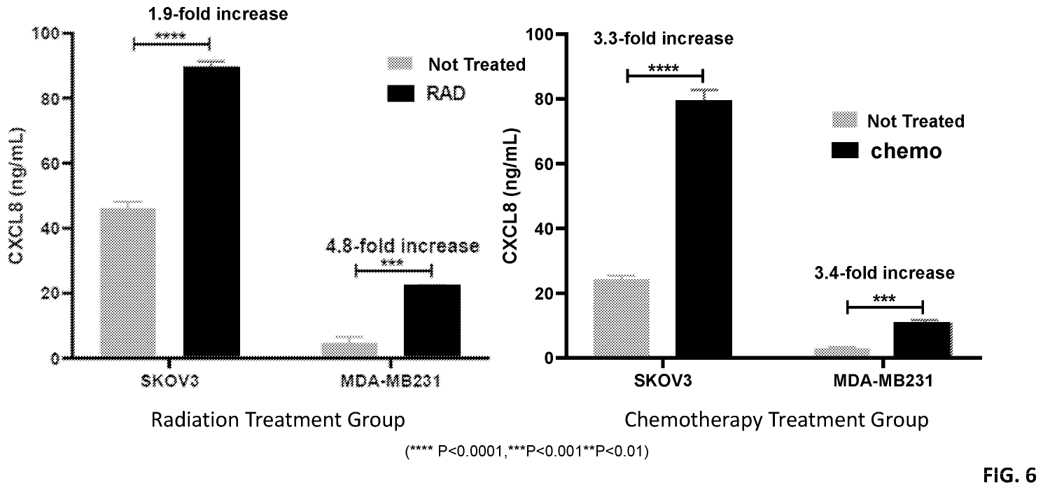

As shown in FIG. 6, ELISA measurement of increased secretion of CXCL8 by the indicated solid tumor cell lines with irradiation (FIG. 6, left panel) and chemotherapy (FIG. 6, right panel) preconditioning is further demonstrated, showing 3.3-fold over baseline control for chemotherapy (24 ng/ml increased to 79 ng/ml for SKOV3; 2.9 ng/ml increased to 12.5 ng/ml for MDA-MB-231), and a 1.9- to 4.8-fold increase for radiation. As such, it is shown that preconditioning or sensitizing with chemo- and/or radio-therapy augments/increases secretion and/or surface expression of one or more CXCR2 ligands in multiple solid tumor indications.

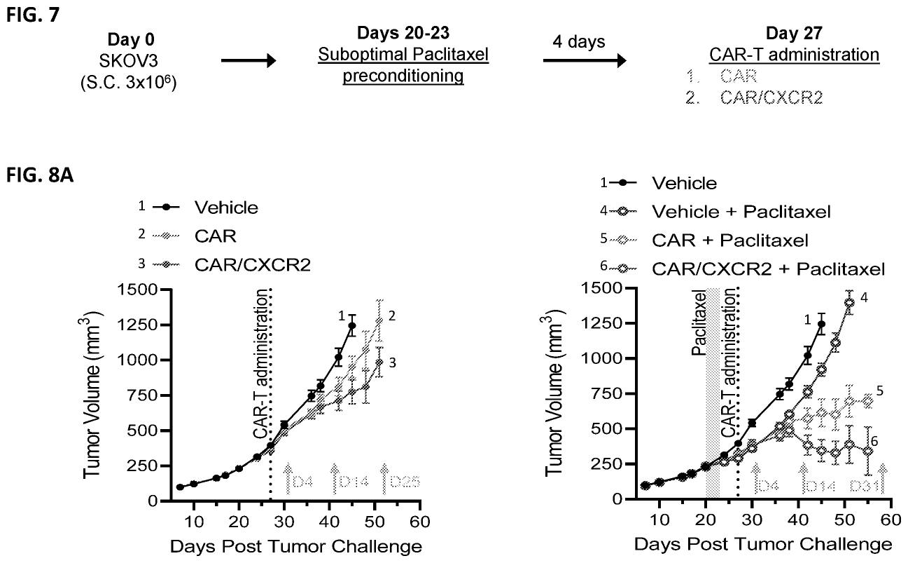

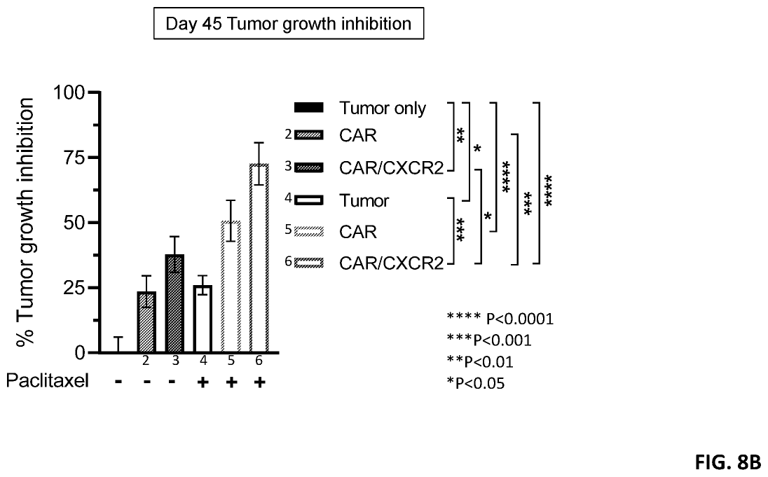

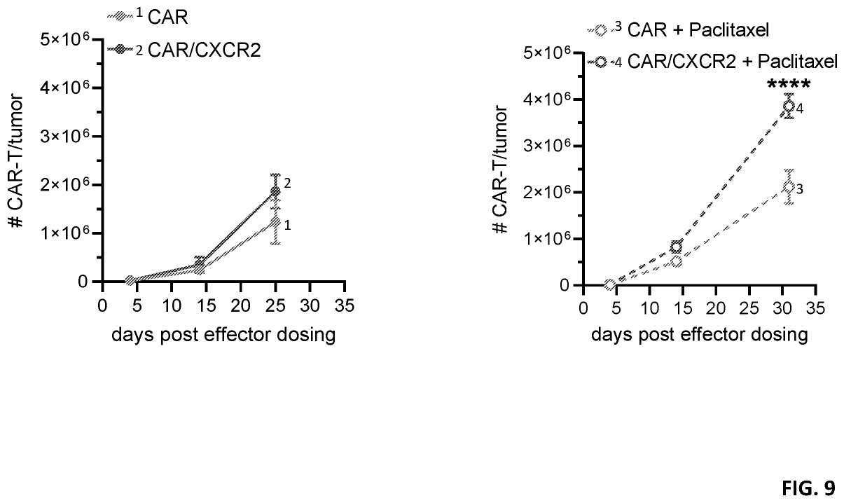

Subsequently, chemotherapeutic preconditioning of tumor cells was evaluated for improved CAR-T intratumoral infiltration/retention and tumor growth control/inhibition. FIG. 7 shows an exemplary experimental design for an in vivo evaluation of CXCR2⁻ and CXCR2⁺ CAR-T cells with or without preconditioning using the chemotherapeutic agent paclitaxel. Briefly, 3×10⁶ SKOV3 tumor cells were subcutaneously injected into NSG mice and 20 days later, half of the tumor bearing mice were administered a suboptimal (preconditioning) dose of paclitaxel daily until Day 23 to increase chemokine (CXCR2 ligand) expression in the tumor cells. At about D27, CXCR2⁺ and CXCR2⁻ CAR-T cells were administered to the preconditioned and control mice. Tumor measurements for CXCR2⁺ and CXCR2⁻ CAR-T cell treated mice without (FIG. 8A, left panel) and with (FIG. 8A, right panel) in vivo paclitaxel preconditioning demonstrate that with preconditioning CXCR2⁺ CAR-T cells induced enhanced tumor control due to a synergistic promotion of CXCR2⁺ CAR-T cell infiltration by CXCR2 overexpression in the CAR-T cells and CXCR2 ligand upregulation in tumor cells. In addition, tumor growth inhibition (TGI) was calculated at Day 45 for all treatment groups, which demonstrated improved TGI by the heterogenous CXCR2-expressing primary CAR-T cells, as compared to CXCR2⁻ primary CAR-T cells (FIG. 8B). Intratumoral CAR-T infiltration and retention of CXCR2⁺ and CXCR2⁻ CAR-T cells into tumors was evaluated by flow cytometry for effector groups with and without preconditioning, demonstrating enhanced infiltration by CXCR2⁺ primary CAR-T cells, as compared to CXCR2⁻ primary CAR-T cells. As shown in FIG. 9, preconditioning and CXCR2 overexpression synergistically improved CAR-T cell tumor infiltration and prolonged CAR-T cell retention in the tumor.

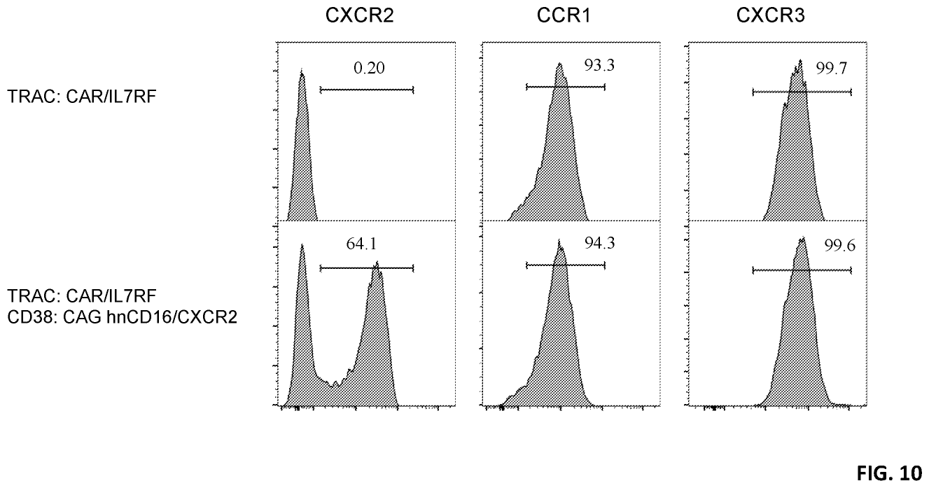

Next, iPSC-derived CAR-T cells expressing (i) TRAC_CAR/IL7RF, or (ii) TRAC_CAR/IL7RF and CD38_hnCD16/CXCR2 were profiled for chemokine receptor expression. The CD38⁻ engineered CAR iT cells demonstrated high levels of CXCR2 expression with 64% of cells expressing high levels of CXCR2, compared to 0.20% in CXCR2⁻ CAR iT cells (FIG. 10, left panels). However, CCR1 and CXCR3, chemokine signaling receptors often important for T cell infiltration into solid tumors, remained unaffected in CXCR2⁺ CAR iT cells compared to CXCR2⁻ CAR iT cells (FIG. 10, middle and right panels; CCR1: 94.3% vs 93.3%; and CXCR3: 99.6% vs 99.7%, respectively).

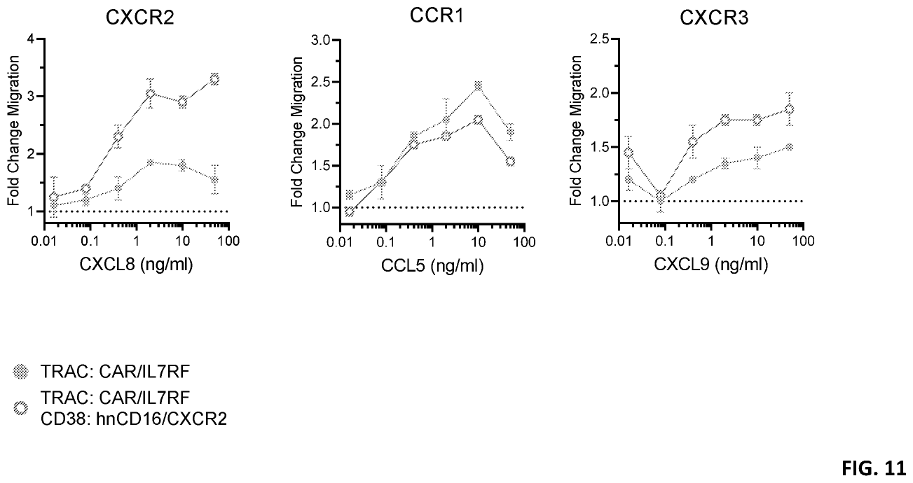

Functional expression of engineered CXCR2 was determined via transwell migration assays. CXCR2⁺ and CXCR2⁻ CAR iT cells were plated into a 5 μm transwell insert and chemokines (CXCL8, CCL5, or CXCL9) of varying dilutions (16 pg/ml-50 ng/ml) were added to the bottom chamber. After 3 hours of culture, specific migration of the iT cells was calculated. As shown in FIG. 11, left panel, CXCR2⁺ CAR iT cells functionally migrated in a dose-responsive manner to varying dilutions of CXCL8, a CXCR2 ligand, whereas sensitivities of CXCR2⁺ CAR iT cells to CCL5 (a CCR1 ligand) and CXCL9 (a CXCR3 ligand) were unaffected (FIG. 11, middle and right panels). These data demonstrate that engineering of CXCR2 into iPSC-derived CAR T cells enables the cells' functional migration to CXCR2 ligands, facilitating improved infiltration and retention into solid tumors and enhanced tumor clearance.

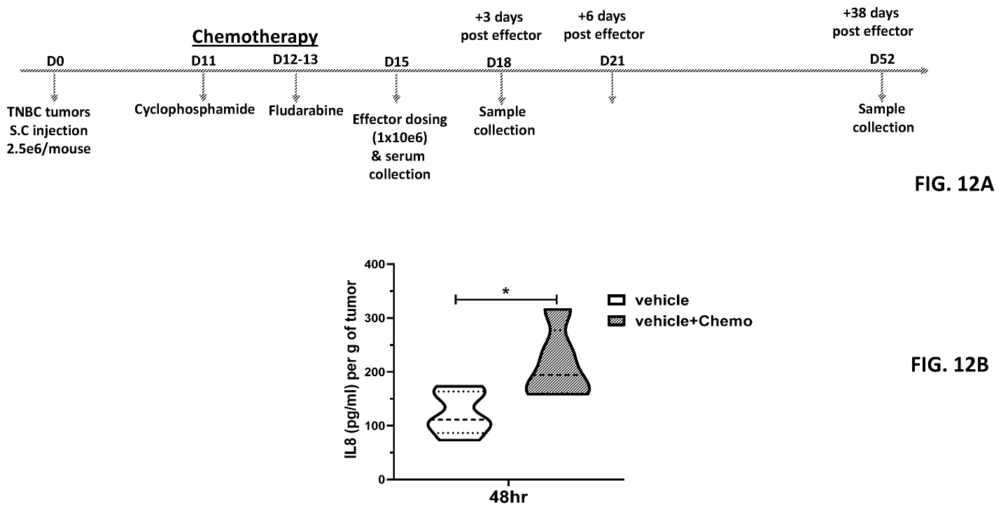

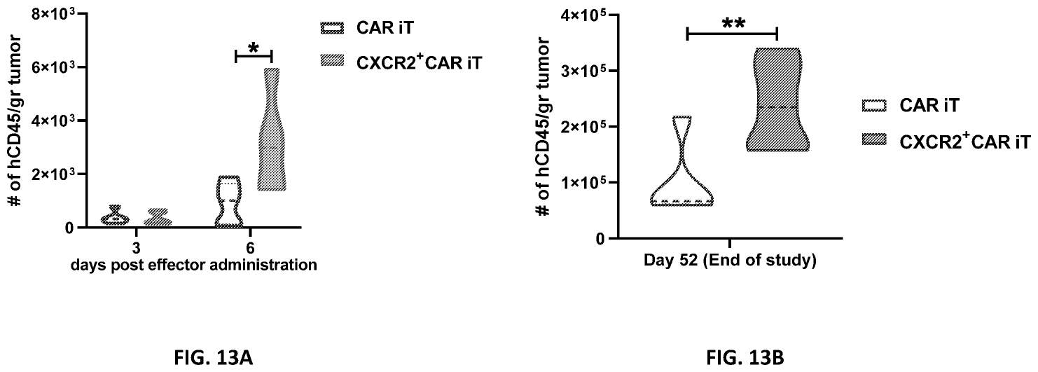

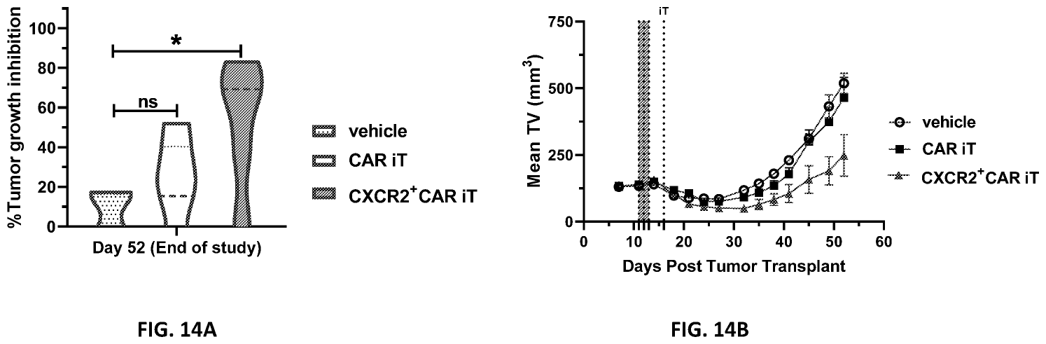

Chemotherapeutic preconditioning of tumor cells was further evaluated for improved CAR-T intratumoral infiltration/retention and tumor growth control/inhibition in a stress test, low effector dose and aggressive xenograft model. FIG. 12A shows an exemplary experimental design for the in vivo evaluation of CXCR2⁺ and CXCR2⁺ CAR-iT cells with chemotherapy (Cyclophosphamide/Fludarabine) preconditioning. Briefly, 2.5e6 triple negative breast cancer (TNBC) tumor cells were subcutaneously injected into NSG mice on Day 0. On Day 11, half of the tumor bearing mice were administered a suboptimal (preconditioning) dose of Cyclophosphamide, followed by suboptimal (preconditioning) doses of Fludarabine on Days 12-13. On D15, 10e6 CXCR2⁺ and CXCR2⁻ CAR-iT cells were administered to the preconditioned and control mice, and serum of each was collected for assessment of CXCL8 levels post chemo-preconditioning (FIG. 12B). Intratumoral CAR-iT infiltration and retention was evaluated by flow cytometry in tumors collected at D18 and D21, corresponding to D3 and D6 post effector administration (FIG. 13A) to show effector cell infiltration after administration, and on D52 (FIG. 13B) to show the number of remaining effector cells as a result of cell retention. Tumor growth inhibition (TGI) was evaluated and compared at Day 52 (end of study) among all treatment groups, which demonstrated improved TGI by the CXCR2⁺ CAR-iT cells, as compared to CXCR2⁻ CAR-T cells (FIG. 14A). Tumor volume measurements (avg. +/−SEM) for CXCR2⁺ and CXCR2⁻ CAR-iT cell treated mice (FIG. 14B) demonstrate that with preconditioning tumor cells for increased CXCL8 expression induced enhanced tumor control due to CXCR2 ligand upregulation in tumor cells.

Collectively, these data demonstrate that delivering the ideal chemokine/chemokine receptor match between tumors and effector cells by precise and rational engineering of unique chemokine receptors in effector cells, and optionally further by tumor cell preconditioning, can improve and enhance effector trafficking and infiltration to, and effector cell retention at, tumor sites and thereby increasing tumor targeting and killing, especially in solid tumors, by the effector cells.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cell or a population thereof, wherein
(i) the cell is (a) an immune cell; (b) an induced pluripotent cell (iPSC); or (c) a derivative effector cell obtained from differentiating the iPSC; and
(ii) the cell comprises:
(1) an exogenous polynucleotide encoding a CXCR2; and optionally
(2) a chimeric antigen receptor (CAR).

2. The cell or a population thereof of claim 1, wherein the iPSC is a clonal iPSC, a single cell dissociated iPSC, an iPSC cell line cell, or an iPSC master cell bank (MCB) cell; or wherein the derivative cell comprises (i) a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, or a derivative B lineage cell; or (ii) the derivative cell comprises a derivative effector cell having one or more functional features that are not present in a counterpart primary T, NK, NKT, and/or B cell.

3. The cell or population thereof of claim 1, wherein the cell further comprises one or more of:
(i) CD38 knockout;
(ii) HLA-I deficiency and/or HLA-II deficiency;
(iii) introduction of HLA-G or non-cleavable HLA-G;
(iv) an exogenous CD16 or variant thereof,
(v) a cytokine signaling complex comprising a partial or full peptide of a cell surface expressed exogenous cytokine and/or a receptor thereof,
(vi) at least one of the genotypes listed in Table 1;
(vii) disruption of least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(viii) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, an engager, and surface triggering receptor for coupling with an agonist.

4. The cell or population thereof of claim 3, wherein the cell has therapeutic properties comprising one or more of:
(i) increased cytotoxicity;
(ii) improved persistency and/or survival;
(iii) enhanced ability in migrating, and/or activating or recruiting bystander immune cells, to tumor sites;
(iv) improved tumor penetration;
(v) enhanced ability to reduce tumor immunosuppression;
(vi) improved ability in rescuing tumor antigen escape;
(vii) controlled apoptosis;
(viii) enhanced or acquired ADCC; and
(ix) ability to avoid fratricide,
in comparison to its counterpart primary cell obtained from peripheral blood, umbilical cord blood, or any other donor tissues without the same genetic edit(s).

5. The cell or population thereof of claim 3, wherein the exogenous CD16 or a variant thereof comprises at least one of:
(a) a high affinity non-cleavable CD16 (hnCD16);
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

6. The cell of population thereof of any one of claims 1-5, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) co-expressed with another CAR;
(ix) co-expressed with a cytokine signaling complex comprising a partial or full peptide of a cell surface expressed exogenous cytokine and/or a receptor thereof, optionally in separate constructs or in a bi-cistronic construct;
(x) co-expressed with a checkpoint inhibitor, optionally in separate constructs or in a bi-cistronic construct;
(xi) specific to at least one of CD19, BCMA, CD20, CD22, CD38, CD123, HER2, CD52, EGFR, GD2, MICA/B, MSLN, VEGF-R2, PSMA and PDL1; and/or
(xii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, Mucin 1 (Muc-1), Mucin 16 (Muc-16), Mesothelin (MSLN), NKCSI, NKG2D ligands, c-Met, cancer-testis antigen NY-ESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBCI, TRBC2, vascular endothelial growth factor R2 (VEGF-R2), Wilms tumor protein (WT-1), and a pathogen antigen; and optionally,
wherein the CAR of any one of (i) to (xii) is inserted at a TCR locus, and/or is driven by an endogenous promoter of the TCR, and/or the TCR is knocked out by the CAR insertion.

7. The cell or population thereof of claim 3, wherein the cytokine signaling complex:
(a) comprises a partial or full peptide of at least one of IL2, IL4, IL6, IL7, IL9, IL10, IL11, IL12, IL15, IL18, IL21, and respective receptor thereof; or
(b) comprises at least one of:
(i) co-expression of IL15 and IL15Rα by suing a self-cleaving peptide;
(ii) a fusion protein of IL15 and IL15Rα;
(iii) an IL15/IL15Rα fusion protein with intracellular domain of IL15Rα truncated;
(iv) a fusion protein of IL15 and membrane bound Sushi domain of IL15Rα;
(v) a fusion protein of IL15 and IL15Rβ;
(vi) a fusion protein of TL15 and common receptor TC, wherein the common receptor TC is native or modified; and
(vii) a homodimer of IL15Rβ;
wherein any one of (b)(i)-(vii) can be co-expressed with a CAR in separate constructs or in a bi-cistronic construct; or
(c) comprises at least one of:
(i) a fusion protein of IL7 and IL7Rα;
(ii) a fusion protein of IL7 and common receptor TC, wherein the common receptor TC is native or modified; and
(iii) a homodimer of IL7Rβ, wherein any one of (c)(i)-(iii) is optionally co-expressed with a CAR in separate constructs or in a bi-cistronic expression cassette;
and optionally,
(d) is transiently expressed.

8. The cell or population thereof of claim 1, wherein the cell is an NK lineage cell or a T lineage cell, wherein:
(i) the NK lineage cell or the T lineage cell has improved infiltration and/or retention at tumor sites;
(ii) the NK lineage cell is capable of recruiting, and/or migrating T cells to tumor sites; or
(iii) the NK lineage cell or the T lineage cell is capable of reducing tumor immunosuppression in the presence of one or more checkpoint inhibitors.

9. The cell or population thereof of claim 6 or 8, wherein the checkpoint inhibitors are antagonists to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR.

10. The cell or population thereof of claim 9, wherein the checkpoint inhibitors comprise:
(a) one or more of atezolizumab, avelumab, durvalumab, ipilimumab, IPH4102, IPH43, IPH33, lirimumab, monalizumab, nivolumab, pembrolizumab, and their derivatives or functional equivalents; or
(b) at least one of atezolizumab, nivolumab, and pembrolizumab.

11. The cell of population thereof of any one of claims 1-5, wherein the cell comprises:
(i) one or more exogenous polynucleotides integrated in a safe harbor locus or a selected gene locus; or
(ii) more than two exogenous polynucleotides integrated in different safe harbor loci or two or more selected gene loci.

12. The cell or population thereof of claim 11, wherein the safe harbor locus comprises at least one of AAVS1, CCR5, ROSA26, collagen, HTRP, H11, GAPDH, or RUNX1; or wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CD69, CD71, CIS, CBL-B, SOCS2, PD1, CTLA4, LAG3, TIM3, or TIGIT; and/or wherein the integration of the exogenous polynucleotides knocks out expression of the gene in the locus.

13. The cell or population thereof of claim 12, wherein the TCR locus is a constant region of TCR alpha and/or TCR beta.

14. A method for improving T lineage cell migration and/or tumor cell control and clearance, the method comprising:
obtaining a T lineage cell comprising an exogenous polynucleotide encoding a CXCR2, and
expanding the T lineage cell, thereby producing a T lineage cell population having improved cell migration and/or tumor cell control and clearance as compared to counterpart cells without the exogenous polynucleotide encoding the CXCR2, wherein the T lineage cell optionally further comprises a chimeric antigen receptor (CAR).

15. The method of claim 14, wherein the step of obtaining comprises (I) or (II):
(I)
(i) engineering an induced pluripotent cell (iPSC) to produce a genomically edited iPSC that comprises one or more exogenous polynucleotides encoding the CXCR2 and optionally a CAR; and
(ii) differentiating the genomically edited iPSC to a derivative T lineage cell comprising the CXCR2 and optionally the CAR; or
(II)
engineering a T lineage cell by introducing a polynucleotide encoding the CXCR2 to the T lineage cell to produce a genomically edited T lineage cell that comprises the CXCR2 and optionally the CAR.

16. The method of claim 15, wherein the genomically edited iPSC further comprises one or more edits resulting in:
(i) CD38 knockout;
(ii) HLA-I deficiency and/or HLA-II deficiency;
(iii) introduction of HLA-G or non-cleavable HLA-G;
(iv) introduction of an exogenous CD16 or a variant thereof,
(v) a cytokine signaling complex comprising a partial or full peptide of a cell surface expressed exogenous cytokine and/or a receptor thereof,
(vi) at least one of the genotypes listed in Table 1;
(vii) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(viii) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, an engager, and surface triggering receptor for coupling with an agonist, in comparison to its counterpart primary cell obtained from peripheral blood, umbilical cord blood, or any other donor tissues without the same genomic edit(s).

17. The method of any one of claims 14-16, wherein the improved cell migration and/or tumor cell control and clearance is in vitro and/or in vivo.

18. A method of improving CAR-T cell in vivo antitumor function according to the method of any one of claims 14-17.

19. A composition comprising the cell or population thereof of any one of the claims 1-13.

20. The composition of claim 19, further comprising one or more therapeutic agents.

21. The composition of claim 20, wherein the one or more therapeutic agents comprise a peptide, a cytokine, a checkpoint inhibitor, an effector, an antibody or functional variant or fragment thereof, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD).

22. The composition of claim 21, wherein:
(a) the checkpoint inhibitor comprises:
(i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR;
(ii) one or more of atezolizumab, avelumab, durvalumab, ipilimumab, IPH4102, IPH43, IPH33, lirimumab, monalizumab, nivolumab, pembrolizumab, and their derivatives or functional equivalents; or
(iii) at least one of atezolizumab, nivolumab, and pembrolizumab; or
(b) the therapeutic agents comprise one or more of venetoclax, azacitidine, and pomalidomide.

23. The composition of claim 21, wherein the antibody, or functional variant or fragment thereof comprises:
(a) an anti-CD20, an anti-HER2, an anti-CD52, an anti-EGFR, an anti-CD123, an anti-GD2, an anti-PDL1, and/or an anti-CD38 antibody;
(b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or
(c) daratumumab, and wherein the derivative effector cell comprises a CD38 knockout, and optionally an expression of CD16 or a variant thereof.

24. The composition of claim 19, further comprising a sensitizing agent.

25. The composition of claim 24, wherein the sensitizing agent comprises at least one of a chemotherapeutic agent, external beam radiation, brachytherapy, and a radiopharmaceutical.

26. The composition of claim 24 or 25, wherein the sensitizing agent increases chemokine secretion and/or surface expression by a tumor cell upon contact therewith.

27. The composition of claim 24, wherein the sensitizing agent comprises:
(i) at least one of calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iorn-59, krypton-81m, lutetium-177, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90;
(ii) paclitaxel; or
(iii) cyclophosphamide and fludarabine (Cy/Flu).

28. Therapeutic use of the composition of any one of the claims 19-27 by introducing the composition to a subject suitable for adoptive cell therapy, wherein the subject has an autoimmune disorder, a hematological malignancy, a solid tumor, cancer, or a viral infection.

29. A master cell bank (MCB) comprising a clonal iPSC of any one of the claims 1-13.

30. A method of manufacturing a derivative effector cell comprising a CXCR2, wherein the method comprises differentiating a genetically engineered iPSC to the derivative effector cell, wherein the genetically engineered iPSC comprises an exogenous polynucleotide encoding the CXCR2 and optionally one or more edits resulting in:
(i) a CAR;
(ii) exogenous CD16 or a variant thereof;
(iii) CD38 knockout;
(iv) HLA-I deficiency and/or HLA-II deficiency;
(v) introduction of HLA-G or non-cleavable HLA-G;
(vi) a cytokine signaling complex comprising a partial or full peptide of a cell surface expressed exogenous cytokine and/or a receptor thereof,
(vii) at least one of the genotypes listed in Table 1;
(viii) disruption of least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(ix) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, an engager, and surface triggering receptor for coupling with an agonist.

31. The method of claim 30, wherein the iPSC is a clonal iPSC, a single cell dissociated iPSC, an iPSC cell line cell, or an iPSC master cell bank (MCB) cell; or wherein the derivative cell comprises (i) a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, or a derivative B lineage cell; or (ii) the derivative cell comprises a derivative effector cell having one or more functional features that are not present in a counterpart primary T, NK, NKT, and/or B cell.

32. The method of claim 30, wherein the exogenous CD16 or variant thereof comprises at least one of:
(a) a high affinity non-cleavable CD16 (hnCD16);
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

33. The method of claim 30, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) co-expressed with another CAR;
(ix) co-expressed with a cytokine signaling complex comprising a partial or full peptide of a cell surface expressed exogenous cytokine and/or a receptor thereof, optionally in separate constructs or in a bi-cistronic construct;
(x) co-expressed with a checkpoint inhibitor, optionally in separate constructs or in a bi-cistronic construct;
(xi) specific to at least one of CD19, BCMA, CD20, CD22, CD38, CD123, HER2, CD52, EGFR, GD2, MICA/B, MSLN, VEGF-R2, PSMA and PDL1; and/or
(xii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), K-light chain, kinase insert domain receptor (KDR), Lewis A (CA19.9), Lewis Y (LeY), L1 cell adhesion molecule (L1-CAM), LILRB2, melanoma antigen family A1 (MAGE-A1), MICA/B, Mucin 1 (Muc-1), Mucin 16 (Muc-16), Mesothelin (MSLN), NKCSI, NKG2D ligands, c-Met, cancer-testis antigen NY-ESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBCI, TRBC2, vascular endothelial growth factor R2 (VEGF-R2), Wilms tumor protein (WT-1), and a pathogen antigen; and optionally,
wherein the CAR of any one of (i) to (xii) is inserted at a TCR locus, and/or is driven by an endogenous promoter of TCR, and/or the TCR is knocked out by the CAR insertion.

34. The method of claim 30, further comprising genomically engineering a clonal iPSC to knock-in a polynucleotide encoding the CXCR2; and optionally:
(i) to knock out CD38,
(ii) to knock out B2M and/or CIITA,
(iii) to knock out one or both CD58 and CD54, and/or
(iv) to introduce one or more of HLA-G or non-cleavable HLA-G, an exogenous CD16 or a variant thereof, a CAR, and/or a cytokine signaling complex comprising a partial or full peptide of a cell surface expressed exogenous cytokine and/or a receptor thereof.

35. The method of claim 34, wherein the genomic engineering comprises targeted editing.

36. The method of claim 35, wherein the targeted editing comprises deletion, insertion, or in/del, and wherein the targeted editing is carried out by CRISPR, ZFN, TALEN, homing nuclease, homology recombination, or any other functional variation of these methods.

37. A method of producing a clonal master engineered iPSC line using CRISPR, ZFN, or TALEN mediated editing of clonal iPSCs, wherein the editing comprises a knock-in of a polynucleotide encoding a CXCR2 and optionally a CAR, thereby producing the engineered iPSCs.

38. The method of claim 37:
(a) wherein the editing of clonal iPSCs further comprises knocking out TCR, or
(b) wherein the CXCR2 is inserted at one of the gene loci comprising: B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and wherein the insertion knocks out expression of the gene in the locus.

39. The method of claim 37, further comprising sorting the engineered iPSCs to obtain single cell dissociated iPSCs comprising the polynucleotide encoding the CXCR2 and optionally the CAR.

40. The method of claim 39, further comprising expanding the single cell dissociated iPSCs to produce the clonal engineered iPSC population.

41. The method of claim 40, further comprising cryopreserving the produced clonal master engineered iPSC line.

42. The method of any one of claims 38-41, the method further comprising analyzing off-target edits and/or karyotype of the engineered iPSCs.

43. A clonal master engineered iPSC line produced using the methods of claims 37-42.

44. A method of treating a disease or a condition comprising administering to a subject in need thereof the composition of any one of claims 19-27.

45. The method of claim 44, wherein the method comprises administering to the subject in need thereof the cell or population thereof of any one of claims 1-13.

46. The method of claim 45, wherein the method further comprises administering a sensitizing agent to the subject, thereby preconditioning tumor cells in the subject.

47. The method of claim of 45 or 46, wherein the method further comprises administering one or more therapeutic agents to the subject.

48. A method of treating a subject comprising:
(a) administering a sensitizing agent to the subject to precondition tumor cells in the subject; and
(b) administering the cell or population thereof of any one of claims 1-13 to the subject following administration of the sensitizing agent,
wherein the subject has an autoimmune disorder; a hematological malignancy; a solid tumor; cancer, or a virus infection.

49. The method of claim 48, wherein the sensitizing agent comprises at least one of a chemotherapeutic agent, external beam radiation, brachytherapy, and a radiopharmaceutical.

50. The method of claim 48, wherein the sensitizing agent increases secretion and/or surface expression of a chemokine that binds to CXCR2, by a tumor cell upon contact therewith.

51. The method of claim 49, wherein the sensitizing agent comprises:
(i) at least one of x-ray radiation, gamma radiation, photon radiation, proton radiation, and neutron radiation; or
(ii) at least one of calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iorn-59, krypton-81m, lutetium-177, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90; or
(iii) paclitaxel; or
(iv) cyclophosphamide and fludarabine (Cy/Flu).

52. The method of claim 48, further comprising administering one or more therapeutic agents.

53. The method of claim 52, wherein the one or more therapeutic agents comprise a peptide, a cytokine, a checkpoint inhibitor, an effector, an antibody or functional variant or fragment thereof, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD).

54. The method of claim 53, wherein:
(a) the checkpoint inhibitor comprises:
(i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR;
(ii) one or more of atezolizumab, avelumab, durvalumab, ipilimumab, IPH4102, IPH43, IPH33, lirimumab, monalizumab, nivolumab, pembrolizumab, and their derivatives or functional equivalents; or
(iii) at least one of atezolizumab, nivolumab, and pembrolizumab; or
(b) the therapeutic agents comprise one or more of venetoclax, azacitidine, and pomalidomide.

55. The method of claim 53, wherein the antibody, or functional variant or fragment thereof comprises:
(a) an anti-CD20, an anti-HER2, an anti-CD52, an anti-EGFR, an anti-CD123, an anti-GD2, an anti-PDL1, and/or an anti-CD38 antibody;
(b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or
(c) daratumumab, and wherein the derivative effector cell comprises a CD38 knockout, and optionally an expression of CD16 or a variant thereof.


SEQUENCE LISTING (22 sequences)

SEQ ID NO: 1 - protein, 360 aa, synthetic construct
  REGION 1..360: note = Synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 2 - protein, 15 aa, synthetic construct
  REGION 1..15: note = Synthetic construct
MEDFNMESDSFEDFW

SEQ ID NO: 3 - protein, 200 aa, synthetic construct
  REGION 1..200: note = Synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDM

SEQ ID NO: 4 - protein, 135 aa, synthetic construct
  REGION 1..135: note = Synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGIL

SEQ ID NO: 5 - protein, 172 aa, synthetic construct
  REGION 1..172: note = Synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGL

SEQ ID NO: 6 - protein, 138 aa, synthetic construct
  REGION 1..138: note = Synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLA

SEQ ID NO: 7 - protein, 39 aa, synthetic construct
  REGION 1..39: note = Synthetic construct
IEVMYPPPYLDNEKSNGTIIHVKGKHLCPSPLFPGPSKP

SEQ ID NO: 8 - protein, 88 aa, synthetic construct
  REGION 1..88: note = Synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFL

SEQ ID NO: 9 - protein, 89 aa, synthetic construct
  REGION 1..89: note = Synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLT

SEQ ID NO: 10 - protein, 129 aa, synthetic construct
  REGION 1..129: note = Synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
  REGION 1..229: note = Synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 153 aa, synthetic construct
  REGION 1..153: note = Synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 13 - protein, 219 aa, synthetic construct
  REGION 1..219: note = Synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 14 - protein, 263 aa, synthetic construct
  REGION 1..263: note = Synthetic construct
SNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 308 aa, synthetic construct
  REGION 1..308: note = Synthetic construct
TTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 340 aa, synthetic construct
  REGION 1..340: note = Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHYQVSFCLVMVLLFAVDTGLYFSVKTNIRSSTRDWKDHKFKWRKDPQDK

SEQ ID NO: 17 - protein, 336 aa, synthetic construct
  REGION 1..336: note = Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLFFPPGYQVSFCLVMVLLFAVDTGLYFSVKTNIRSSTRDWKDHKFKWRKDPQDK

SEQ ID NO: 18 - protein, 335 aa, synthetic construct
  REGION 1..335: note = Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 19 - protein, 379 aa, synthetic construct
  REGION 1..379: note = Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQ

SEQ ID NO: 20 - protein, 375 aa, synthetic construct
  REGION 1..375: note = Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYL

SEQ ID NO: 21 - protein, 242 aa, synthetic construct
  REGION 1..242: note = Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIR

SEQ ID NO: 22 - protein, 635 aa, synthetic construct
  REGION 1..635: note = Synthetic construct
MDWTWILFLVAAATRVHSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSGGGGSGGGGSGGGGSGGGSLQESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ